(19) United States
(12) Patent Application Publication
(10) Pub. No.: US 2003/0134787 A1
(43) Pub. Date: Jul. 17, 2003

(54) Conjugate heat shock protein-binding peptides

(76) Inventors: James E. Rothman, New York, NY (US); Mark Mayhew, Tarrytown, NY (US); Mee H. Hoe, New York, NY (US); Alan Houghton, New York, NY (US); Ulrich Hartl, Munich (DE); Ouathek Ouerfelli, New York, NY (US); Yoichi Moroi, New York, NY (US)

Correspondence Address: KENYON & KENYON, ONE BROADWAY, NEW YORK, NY, 10004, US

(21) Appl. No.: 10/052,578

(22) Filed: Jan. 17, 2002

Related U.S. Application Data
(62) Division of application No. 08/961,707, filed on Oct. 31, 1997, now abandoned.

(51) Int. Cl. A61K 38/17; C12Q 1/70; G01N 33/53

(52) U.S. Cl. 514/12; 435/7.1; 435/5

(57) ABSTRACT

The present invention relates (i) to conjugate peptides engineered to noncovalently bind to heat shock proteins; (ii) to compositions comprising such conjugate peptides, optionally bound to heat shock protein; and (iii) to methods of using such compositions to induce an immune response tin a subject in need of such treatment. It is based, at least in part, on the discovery of tethering molecules which may be used to non-covalently link antigenic peptides to heat shock proteins. The present invention also provides for methods of identifying additional tethers which may be comprised, togethter with antigenic sequences, in conjugate peptides.

### 1. INTRODUCTION

The present invention relates (i) to conjugate peptides engineered to noncovalently bind to heat shock proteins; (ii) to compositions comprising such conjugate peptides, optionally bound to heat shock protein; and (iii) to methods of using such compositions to induce an immune response in a subject in need of such treatment. It is based, at least in part, on the discovery of peptide sequences which may be used to tether antigenic peptides to heat shock proteins. The present invention also provides for methods of identifying additional tethering peptides which may be comprised, together with antigenic sequences, in conjugate molecules.

### 2. BACKGROUND OF THE INVENTION

Heat shock proteins constitute a highly conserved class of proteins selectively expressed in cells under stressful conditions, such as sudden increases in temperature or glucose deprivation. Able to bind to a wide variety of other proteins in their non-native state, heat shock proteins participate in the genesis of these bound proteins, including their synthesis, folding, assembly, disassembly and translocation (Freeman and Morimoto, 1996, EMBO J. 15:2969-2979; Lindquist and Craig, 1988, Annu. Rev. Genet. 22:631-677; Hendrick and Hartl, 1993, Annu. Rev. Biochem. 62:349-384). Because they guide other proteins through the biosynthetic pathway, heat shock proteins are said to function as “molecular chaperones” (Frydman et al., 1994, Nature 370:111-117; Hendrick and Hartl, Annu. Rev. Biochem. 62:349-384; Hartl, 1996, Nature 381:571-580). Induction during stress is consistent with their chaperone function; for example, dnaK, the Escherichia coli hsp70 homolog, is able to reactivate heat-inactivated RNA polymerase (Ziemienowicz et al., 1993, J. Biol. Chem. 268:25425-25341).

The heat shock protein gp96 resides in the endoplasmic reticulum, targeted there by an amino-terminal signal sequence and retained by a carboxy-terminal KDEL amino acid motif (which promotes endoplasmic reticulum recapture; Srivastava et al., 1987, Proc. Natl. Acad. Sci. U.S.A. 84:3807-3811). Found in higher eukaryotes but not in Drosophila or yeast, gp96 appears to have evolved relatively recently, perhaps by a duplication of the gene encoding the cytosolic heat shock protein hsp9o, to which it is highly related (Li and Srivastava, 1993, EMBO J. 12:3143-3151; identity between human hsp90 and murine gp96 is about 48 percent). It has been proposed that gp96 may assist in the assembly of multi-subunit proteins in the endoplasmic reticulum (Wiech et al., 1992, Nature 358:169-170). Indeed, gp96 has been observed to associate with unassembled immunoglobulin chains, major histocompatability class II molecules, and a mutant glycoprotein B from Herpes simplex virus (Melnick et al., 1992, J. Biol. Chem. 267:21303-21306; Melnick et al., 1994, Nature 370:373-375; Schaiff et al., 1992, J. Exp. Med. 176:657-666; Ramakrishnan et al., 1995, DNA and Cell Biol. 14:373-384). Further, expression of gp96 is induced by conditions which result in the accumulation of unfolded proteins in the endoplasmic reticulum (Kozutsumi et al., 1988, Nature 332:462-464). It has been reported that gp96 appears to have ATPase activity (Li and Srivastava, 1993, EMBO J. 12:3143-3151), but this observation has been questioned (Wearsch and Nicchitta, 1997, J. Biol. Chem. 272:5152-5156).

Unlike gp96, hsp90 lacks the signal peptide and KDEL sequence associated with localization in the endoplasmic reticulum, residing, instead, in the cytosol. Although hsp90 has not been detected as a component of the translational machinery (Frydmann et al., 1994, Nature 370:111-116), it has been reported to be highly effective in converting a denatured protein, in the absence of nucleotides such as ATP or ADP, to a “folding competent” state which can subsequently be refolded upon addition of hsp70, hdj-1 and nucleotide (Freeman and Morimoto, 1996, EMBO J. 15:2969-2979; Schneider et al., 1996, Proc. Natl. Acad. Sci. U.S.A. 93: 14536-14541). Hsp90 has been observed to serve as a chaperone to a number of biologically highly relevant proteins, including steroid aporeceptors, tubulin, oncogenic tyrosine kinases, and cellular serine-threonine kinases (Rose et al., 1987, Biochemistry 26:6583-6587; Sanchez et al., 1988, Mol. Endocrinol. 2:756-760; Miyata and Yahara, 1992, J. Biol. Chem. 267:7042-7047; Doyle and Bishop, 1993, Genes Dev. 7:633-638; Smith and Toft, 1993, Mol. Endocrinol. 7:4-11; Xu and Lindquist, 1993, Proc. Natl. Acad. Sci. U.S.A. 90:7074-7078; Stancato et al., 1993, J. Biol. Chem. 268: 21711-21716; Cuttforth and Rubin, 1994, Cell 77:1027-1035; Pratt and Welsh, 1994, Semin. Cell Biol. 5:83-93; Wartmann and Davis, 1994, J. Biol. Chem. 269:6695-6701; Nathan and Lindquist, 1995, Mol. Cell. Biol. 15:3917-3925; Redmond et al., 1989, Eur. J. Cell. Biol. 50:66-75). Hsp90 has been observed to function in concert with other proteins, some of which may act as true chaperones, others serving only as accessories; for example, cellular assembly of the progesterone receptor has been reported to involve hsp90 and seven other proteins (Smith et al., 1995, Mol. Cell. Biol. 15:6804-6812).

Hsp90 has been implicated in the mechanism of reversion of transformation by the antibiotics geldanamycin and herbimycin A (Whitesell et al., 1994, Proc. Natl. Acad. Sci. U.S.A. 91:8324-8328; for structures see FIG. 9A). These antibiotics are members of a class of compounds known as benzoquinone ansamycins, derived from actinomycetes and originally isolated for their herbicidal activity (Omura et al., 1979, J. Antibiotics 32:255-261). Exposure to herbimycin A and geldanamycin was observed to revert the morphology of fibroblasts transformed via various oncogenic tyrosine kinases, including src, fyn, Ick, bcr-abl, and erbB2 (Uehara et al., 1988, Virology 164:294-298); as a result, these compounds have been (rather erroneously, see infra) referred to as tyrosine kinase inhibitors, and have been tested as anti-cancer drugs (Yoneda et al., 1993, J. Clin. Invest. 91:2791-2795; Honma et al., 1995, Int. J. Cancer 60:685-688).

It was reported that herbimycin A treatment of Rous sarcoma virus-transformed cells resulted in reduced kinase activity and increased turnover of the tyrosine kinase p₆₀^{v-src} (Uehara et al., 1989, Cancer Res. 49:780-785). However, benzoquinone ansamycins were subsequently found to have no direct effect on tyrosine kinase activity (Whitesell et al., 1992, Cancer Res. 52:1721-1728); rather, their mechanism of action appears to involve inhibition of hsp90/tyrosine kinase heteroprotein complex formation and consequent increased turnover of p60^{v-src} (Whitesell et al., 1994, Proc. Natl. Acad. Sci. U.S.A. 91:8324-8328). These drugs have also been shown to interfere with the chaperone function of hsp90 outside of the tyrosine kinase context; Smith et al. (1995, Mol. Cell. Biol. 15:6804-6812) report that geldanamycin arrests progesterone receptor assembly at an intermediate step.

Inoculation with heat shock protein prepared from tumors of experimental animals has been shown to induce immune responses in a tumor-specific manner; that is to say, heat shock protein gp96 purified from a particular tumor could induce an immune response which would inhibit the growth of cells from the identical tumor of origin, but not other tumors, regardless of relatedness (Srivastava and Maki, 1991, Curr. Topics Microbiol. 167:109-123). The source of the tumor-specific immunogenicity has not been confirmed. Genes encoding heat shock proteins have not been found to exhibit tumor-specific DNA polymorphism (Srivastava and Udono, 1994, Curr. Opin. Immunol. 6:728-732). High-resolution gel electrophoresis has indicated that tumor-derived gp96 may be heterogeneous at the molecular level; evidence suggests that the source of this heterogeneity may be populations of small peptides adherent to the heat shock protein, which may number in the hundreds (Feldweg and Srivastava, 1995, Int. J. Cancer 63:310-314). Indeed, an antigenic peptide of vesicular stomatitis virus has been shown to associate with gp96 in virus infected cells (Nieland et al., 1996, Proc. Natl. Acad. Sci. U.S.A. 93:6135-6139). It has been suggested that this accumulation of peptides is related to the localization of gp96 in the endoplasmic reticulum, where it may act as a peptide acceptor and accessory to peptide loading of major histocompatability complex class I molecules (Li and Srivastava, 1993, EMBO J. 12:3143-3151; Suto and Srivastava, 1995, Science 269:1585-1588).

The use of heat shock proteins as adjuvants to stimulate an immune response has been proposed (see, for example, Edgington, 1995, Bio/Technol. 13:1442-1444; PCT Application International Publication Number WO 94/29459 by the Whitehead Institute for Biomedical Research, Richard Young, inventor, and references infra). One of the best known adjuvants, Freund's complete adjuvant, contains a mixture of heat shock proteins derived from mycobacteria (the genus of the bacterium which causes tuberculosis); Freund's complete adjuvant has been used for years to boost the immune response to non-mycobacterial antigens. A number of references suggest, inter alia, the use of isolated mycobacterial heat shock proteins for a similar purpose, including vaccination against tuberculosis itself (Lukacs et al., 1993, J. Exp. Med. 178:343-348; Lowrie et al., 1994, Vaccine 12:1537-1540; Silva and Lowrie, 1994, Immunology 82:244-248; Lowrie et al., 1995, J. Cell. Biochem. Suppl. 0(19b):220; Retzlaff et al., 1994, Infect. Immun. 62:5689-5693; PCT Application International Publication No. WO 94/11513 by the Medical Research Council, Colston et al., inventors; PCT Application International Publication No. WO 93/1771 by Biocine Sclavo Spa, Rappuoli et al., inventors).

Other references focus on the ability of heat shock proteins to naturally form associations with antigenic peptides, rather than the classical adjuvant activity (see, for example PCT Application No. PCT/US96/13233 by Sloan-Kettering Institute for Cancer Research, Rothman et al., inventors; Blachere and Srivastava, 1995, Seminars in Cancer Biology 6:349-355; PCT Application International Publication No. WO 95/24923 by Mount Sinai School of Medicine of the City University of New York, Srivastava et al., inventors). In one protocol used by Srivastava in a phase I European clinical trial, cells prepared from a surgically resected tumor were used to prepare gp96, which was then reinoculated into the same patient (Edgington, 1995, Bio/Technol. 13:1442-1444). The fact that a new gp96 preparation must be made for each patient is a significant disadvantage. PCT Application International Publication No. WO 95/24923 (supra) suggests that peptides in heat shock protein complexes may be isolated and then re-incorporated into heat shock protein complexes in vitro. There is no evidence that this time-consuming procedure would be successful beyond the treatment of the patient from which the heat shock protein was derived. Further, the preparation of an effective quantity of heat shock protein requires the harvest, from the patient, of an amount of tissue which not every patient would be able to provide. Moreover, this approach limits the use of heat shock proteins as peptide carriers to those peptides with which a natural association is formed in vivo, and the affinity of such peptides for heat shock protein may be inadequate to produce a desired immune response using complexes generated in vitro.

In attempts to circumvent these limitations, heat shock proteins have been covalently joined to antigenic peptides of choice. For example, it has been reported that a synthetic peptide comprising multiple iterations of NANP (Asn Ala Asn Pro) malarial antigen, chemically crosslinked to glutaraldehyde-fixed mycobacterial heat shock proteins hsp65 or hsp70, was capable of inducing a humoral (antibody based) immune response in mice in the absence of further adjuvant; a similar effect was observed using heat shock protein from the bacterium Escherichia coli (Del Guidice, 1994, Experientia 50:1061-1066; Barrios et al., 1994, Clin. Exp. Immunol. 98:224-228; Barrios et al., 1992, Eur. J. Immunol..22:1365-1372). Cross-linking of synthetic peptide to heat shock protein and possibly glutaraldehyde fixation were required for antibody induction (Barrios et al., 1994, Clin. Exp. Immunol. 98:229-233), and cellular immunity does not appear to be induced. In another example, Young et al., in PCT Application International Publication Number WO 94/29459, discloses fusion proteins in which an antigenic protein is joined to a heat shock protein.

A potential disadvantage of such covalent linkage approaches is that they tend to favor an antibody-based, rather than a cellular, immune response. In such context, the heat shock protein may act as a carrier to promote antibody responses to covalently linked proteins or peptides, a well known adjuvant function of immunogenic proteins. Furthermore, heat shock protein and antigen are irreversibly linked; this may alter the solubility of either protein component, or may create structural distortion which interferes with the association between antigen and critical major histocompatability complex components.

The present invention overcomes these limitations by using conjugate peptides comprising the desired target antigen and also a tether which binds to heat shock proteins without the need for covalent attachment. Rothman et al., in PCT Application No. PCT/US96/13363, discloses such conjugate peptides including a peptide comprising, as a tether, a peptide sequence recognized by Blond-Elguindi et al. (1993, Cell 75:717-218) as binding to the heat shock protein BiP (a member of the hsp70 protein family). The present invention relates to the identification of additional tethers which may be comprised, together with an antigen, into conjugate peptides. In preferred, nonlimiting embodiments of the invention, such tethers may be comprised in conjugate peptides in order to noncovalently link antigen with the heat shock proteins hsp90 and/or gp96. Furthermore, unlike prior art approaches which utilize heat shock proteins in their traditional, adjuvant role, the present invention encompasses the use of heat shock proteins found in the intended host species, including endogenous heat shock proteins.

### 3. SUMMARY OF THE INVENTION

The present invention relates to conjugate peptides comprising (i) a portion which may be bound to a heat shock protein under physiologic conditions, referred to hereafter as the “tether”; and (ii) a portion which is antigenic (hereafter, the “antigenic peptide”). Both peptide and nonpeptide tethers are provided for.

In addition to providing for specific tethers and conjugate peptides, the present invention also relates to methods of identifying further tethers. These methods utilize filamentous phage expression library panning, and are improvements over prior art phage panning protocols in that the methods of the invention (i) simulate conditions found in the native cellular location for peptide/heat shock protein binding; (ii) utilize compounds which facilitate the binding of peptide to heat shock protein, such as ansamycin antibiotics; and/or (iii) isolate regions of heat shock protein which are associated with peptide binding and use said isolated regions as the substrate in a phage panning protocol.

The invention further relates to the use of conjugate peptides in inducing an immune response in a subject. The resulting immune response may be directed toward, for example, a tumor cell or a pathogen, and as such may be used in the prevention or treatment of an infectious or malignant disease. The conjugate peptides of the invention may be administered either together with or, alternatively, without, one or more heat shock proteins. It has been discovered that a conjugate peptide, administered without exogenous heat shock protein, was capable of inducing an immune response.

### 4. DESCRIPTION OF THE DRAWINGS

FIG. 1A-H. (A-G), respectively, show the distribution of amino acids at positions 1-7 of heptapeptides expressed by phage bound to gp96 in the presence of herbimycin A, where the binding buffer used was 20 mM HEPES pH 7.5, 100 mM KCl, 1 mM MgAcetate, and 0.1%, 0.3%, or 0.5% TWEEN 20 depending on the panning round. (H). Amino acid sequences (SEQ ID NOS: —) and corresponding nucleic acid sequences (SEQ ID NOS: —) of certain binding peptides.

FIG. 2A-H. (A-G), respectively, show the distribution of amino acids at positions 1-7 of heptapeptides expressed by phage bound to gp96 in the presence of herbimycin A, where the binding buffer used was 20 mM HEPES pH 7.5, 100 mM KCl, 1 mM DTT, 1 mM MgAcetate, and 0.1%, 0.3%, or 0.5% TWEEN 20 depending on the panning round. (H). Amino acid sequences (SEQ ID NOS —) and corresponding nucleic acid sequences (SEQ ID NOS: —) sequences of certain binding peptides.

FIG. 3A-B. Cytotoxic activity of effector Tcells prepared from mice, immunized once with OVA peptide (SIINFEKL; SEQ ID NO: ) plus TiterMax adjuvant, against OVA-primed EL-4 target cells (A) or unprimed EL-4 control cells (B). In a careful comparison of immune adjuvants, TiterMax was shown previously to be the optimal adjuvant for induction of cytotoxic T cell responses against OVA peptide and other peptides (Dyall et al., 1995, Intemat. Immunol. 7:1205-1212).

FIG. 4A-B. Cytotoxic activity of effector T cells prepared from mice immunized with hsp70 plus OVA-BiP conjugate peptide against OVA-primed EL-4 target cells (A) or unprimed EL-4 control cells (B). Each curve represents data obtained from a single mouse. Mice were either immunized once (solid squares and triangles) or twice (open squares and rectangles).

FIG. 5A-B. Cytotoxic activity of effector T cells prepared from mice immunized once (solid squares and triangles) or twice (open squares and rectangles) with OVA-BiP conjugate peptide (without added adjuvant or hsp70) against OVA-primed EL-4 target cells (A) or unprimed EL-4 control cells (B).

FIG. 6A-B. Cytotoxic activity of effector T cells prepared from mice immunized once (solid squares and triangles) or twice (open squares and rectangles) with TiterMax plus OVA-BiP conjugate peptide against OVA-primed EL-4 target cells (A) or unprimed EL-4 control cells (B).

FIG. 7. Cytotoxic activity of effector T cells prepared from mice immunized once (solid circles) or twice (open squares and diamonds) with OVA-peptide alone.

FIG. 8A-H. Tumor diameters in mice immunized with (A) TiterMax plus OVA-peptide; (B) Hsp70 plus OVA-peptide; (C) TiterMax plus OVA-BiP; (D) Hsp70 plus OVA-BiP; (E) control (no immunization; tumor cells only injected); (F) OVA-peptide alone; or (G) OVA-BiP alone prior to EG7 tumor cell challenge. (H) depicts the average delay of onset of EG7-OVA tumor growth in mice immunized with either OVA peptide only, TiterMax and OVA peptide, Hsp70 and OVA peptide, or Hsp70 or OVA-BiP.

FIG. 9A-D. (A). Structures of geldanamycin (“GDM”) and herbimycin A (“HA”). (B). Reaction of a primary amine with geldanamycin at the carbon 17 position. (C). Comparison of the reactivities of herbimycin A and geldanamycin towards the same nucleophile. (D). Reaction of linker with geldanamycin and herbimycin A, and different products obtained therefrom.

FIG. 10A-F. Conjugation of peptides, via their carboxyl termini, to geldanamycin using a variety of linker molecules. Three pairs of examples are presented in (A-F), which are either schematic (A, C and E) or which specifically utilize the OVA peptide (B, D and F).

FIG. 11A-F. Conjugation of peptides, via their amino termini, to geldanamycin using a variety of linker molecules. Three pairs of examples are presented in (A-F), which are either schematic (A, C and E) or which specifically utilize the OVA peptide.

FIG. 12. Attachment of Fmoc-protected amino acid to TGT and chlorotrityl resins.

FIG. 13A-B. Synthesis of protected peptide on TGT resin to produce a fully protected intermediate which may be used for coupling of geldanamycin at the amino terminus of a peptide.

FIG. 14A-B. (A) Protection of the last amino acid of peptide synthesis with Boc and (B) removal of the protected peptide from TGT resin to produce a peptide with a reactive carboxyl terminus for coupling to geldanamycin.

FIG. 15. Reaction of geldanamycin with the carboxyl terminus of a peptide protected at its amino terminus followed by deprotection using 95% trifluoroacetic acid (“TFA”), 2.5% methylene chloride (CH₂Cl₂) and 2.5% triisopropylsilane (“TIPS”) and purification (using a polyHYDROXYETHYL Aspartamide column.

FIG. 16A-B. Reaction of geldanamycin with the amino terminus of a peptide protected at its carboxy terminus followed by deprotection and purification..

FIG. 17A-C. Conjugate peptides comprising a geldanamycin analog with lower binding affinity for heat shock protein. (A). Preparation of a geldanamycin analog with a known lower affinity for hsp90. (B). Amino terminal conjugate of a low affinity geldanamycin analog. (C). Carboxyl terminal conjugate of a low affinity geldanamycin analog.

FIG. 18. Conjugate peptides comprising antigenic peptide joined to geldanamycin via a variety of cleavable linkers.

FIG. 19A-G. Melanoma tumor growth in mice challenged with the OVA-expressing melanoma cell line MO4 after immunization with either (A) TiterMax plus OVA peptide; (B) Hsp70 and OVA peptide; or (C) Hsp70 and OVA-BiP peptide. (D and E) show tumor growth when either OVA peptide alone (D) or Hsp70 and OVA-BiP (E) were administered 14 days after tumor challenge. (F) depicts the survival ratios of mice immunized seven days before challenge with melanoma cells. (G) depicts the survival ratios of mice immunized seven and fourteen days after challenge with melanoma cells.

### 5. DETAILED DESCRIPTION OF THE INVENTION

For purposes of clarity of presentation, and not by way of limitation, the detailed description of the invention is divided into the following subsections:

(i) methods for identifying tethers;

(ii) conjugate peptides; and

(ill) methods of using conjugate peptides.

### 5.1. METHODS FOR IDENTIFYING TETHERS

The present invention provides for methods for identifying a tether which may be comprised, together with an antigenic peptide, in a conjugate peptide. The conjugate peptide, via the tether, may then associate with a heat shock protein in vitro and/or in vivo.

Identification of suitable tethers may be achieved through the technique of affinity panning, using an expression library such as a filamentous phage expression library, to identify cloned peptides which bind to a heat shock protein. Suitable phage display libraries include, but are not limited to, the “Ph.D. Phage Display Peptide Library Kit” (Catalog #8100, New England BioLabs), the “Ph.D.-12 Phage Display 12-mer Peptide Library” (Catalog #8110, New England BioLabs), the “T7Select Phage Display System” (Novagen, Inc.) (see also, U.S. Pat. No. 5,223,409; 5,403,484; and 5,571,698) and libraries prepared as described in Blond-Elguindi et al. (1993, Cell 75:717-728, citing Cwirla et al., 1990, Proc. Natl. Acad. Sci. U.S.A. 87:6378-6382), which reports the identification of peptides that bind to BiP using phage panning. For example, and not by way of limitation, this technique may be practiced by exposing a phage expression library, each phage displaying a different peptide sequence, to a solid substrate coated with a heat shock protein target (henceforth, the “hsp target”), under conditions which allow the binding of phage to the hsp target. Unbound phage is then washed away, and specifically-bound phage is eluted either using a substance which releases peptide from the hsp target, or by lowering the pH. The eluted pool of phage may then be amplified, and the process may then be repeated (preferably three or four times), using the selected phage. Then, individual clones may be isolated and sequenced to identify the peptides which they contain. The identified peptides may then be synthesized in quantities which allow direct testing of their ability to bind to hsp target.

As a specific, nonlimiting example, the “Ph.D. Phage Display Library” from New England Biolabs may be utilized to identify tethers, using the protocol set forth in the corresponding instruction manual. The “Ph.D. Phage Display Library” is a combinatorial library of random peptide heptamers fuised to a minor coat protein (pIII) of the filamentous coliphage M13. The library consists of 2×10⁹ electroporated sequences, amplified once, to yield an average of approximately 100 copies of each peptide sequence in 10 μl of the phage library. The displayed heptapeptides are expressed directly at the N-terminus of pIII, followed by a short spacer (Gly Gly Gly Ser; SEQ ID NO: ) and the native pIII protein. Affinity panning using this library may be performed as follows. A well (6 mm in diameter) of a 96 well polystyrene microtiter plate may be coated with hsp target by adding 150 μl of a 100-200 μg/ml solution of hsp target in 0.1 M NaHCO₃, pH 8.3-8.6, and swirling until the well surface is completely wet. The plate may then be incubated overnight at 4° C. on a rocker in a humidified container (e.g., the wells may be covered with tape or the plate may be placed in a sealable plastic box lined with damp paper towels). Plates containing wells prepared in this manner may be stored at 4° C. in a humidified container until needed. Immediately prior to use, the coating solution is poured off, and residual solution removed. The well may then be filled with “blocking buffer” (0.1 M NaHCO₃ (pH 8.6), 5 mg/ml bovine serum albumin (BSA), 0.02% NaN₃), and incubated at 4° C. for at least one hour. The blocking solution may then be discarded, and the well washed rapidly about six times with “TBST” [50 mM Tris-HCl (pH 7.5), 150 mM NaCl, 0.1-0.5% (v/v) TWEEN-20 (the percentage of TWEEN-20 may be increased from 0.1% to 0.5% in successive rounds of panning)], working quickly to avoid the well drying out. 2×10¹¹ phage may then be diluted in 100 μl of “binding buffer” (which may be TBST or which may be varied as discussed infra), and pipetted into the coated well. The plate may then be rocked gently, at room temperature or at 37° C., for 10-60 minutes. Then, the phage-containing solution may be discarded, and the well washed about ten times with binding buffer. Next, bound phage may be eluted by adding 100 μl 0.2 M glycine-HCl pH 2.2 and incubating for about ten minutes. The resulting eluate may then be pipetted into a microcentrifuge tube and neutralized with 15 μl 1.5 M Tris pH 8.8-9.1. The eluate may then be amplified by inoculating a mid-log phase culture of ER2537 Escherichia coli (F′ lac^{q}DELTA(lacZ)M15proA+B+/fluA2supEthiDELTA(lac-proAB)DELTA(hsdMS-mcrB)5 (rₖ⁻mₖ⁻McrBC⁻) with the eluted phage, and incubating at 37° C. with vigorous shaking for about 4.5 hours. If small numbers of phage elute from the hsp target, a second round of amplification, using a fresh host cell culture in mid-log phase, may be desirable. The culture may then be transferred to a centrifuge tube and spun for 10 minutes at 10,000 rpm (using, for example, a Sorvall SS-34 rotor) at 4° C. The supernatant may then be transferred to a fresh centrifuge tube and re-spun. The upper 80 percent of the resulting supernatant may then be transferred to a fresh tube, and ⅙ volume of PEG/NaCl (20% (w/v) polyethylene glycol-8000, 2.5 M NaCl) may be added. The phage may then be allowed to precipitate at 4° C. for at least 1 hour, and preferably overnight. The precipitated solution may be centrifuged for 15 minutes at 10,000 rpm at 4° C., after which the supernatant may be decanted, the tube re-spun briefly, and residual supernatant may be removed with a pipet. The resulting pellet may be resuspended in 1 ml TBS (50 mM Tris-HCl (pH 7.5), 150 mM NaCl), which may then be transferred to a microcentrifuge tube and spun for 5 minutes at 4° C. The supernatant may be transferred to a fresh microcentrifuge tube and reprecipitated by adding ⅙ volume PEG/NaCl, incubating on ice for 15-60 minutes, and centrifuging in a microfuge for 10 minutes at 4° C. The supernatant may be discarded, the tube re-spun briefly, and residual supernatant discarded as before. The pellet may be suspended in 200 μl TBS containing 0.02% NaN₃, and the resulting solution microcentrifuged for about one minute to remove any remaining insoluble material. The supernatant constitutes amplified eluate, which may be titered to determine the volume which contains 2×10¹¹ pfu. The amplified eluate may then be used in a second round of biopanning. Preferably, three rounds of biopanning are used to identify phage which specifically bind to hsp target.

The hsp target used for affinity panning may be any heat shock protein or portion thereof, or any fusion protein comprising at least a portion of a heat shock protein. The term “heat shock protein”, as used herein, refers to stress proteins (including homologs thereof expressed constitutively), including, but not limited to, gp96, hsp90, BiP, hsp70, hsp60, hsp40, hsc70, and hsp10. Hsp target may be prepared from a natural source, expressed recombinantly, or chemically synthesized.

For example, recombinant expression of gp96 for use as a hsp target is described in Section 6, infra. cDNAs which may be used to express other heat shock proteins include, but are not limited to, gp96: human: Genebank Accession No. X15187; Maki et al., Proc. Natl. Acad. Sci. U.S.A. 87:5658-5562; mouse: Genebank Accession No. M16370; Srivastava et al., Proc. Natl. Acad. Sci. U.S.A. 84:3807-3811; BiP: human: Genebank Accession No. M19645, Ting et al., 1988, DNA 7:275-286; mouse Genebank Accession No. U16277, Haas et al., 1988, Proc. Natl. Acad. Sci. U.S.A. 85:2250-2254; hsp70: human: Genebank Accession No. M24743, Hunt et al., 1985, Proc. Natl. Acad. Sci. U.S.A. 82:6455-6489; mouse: Genebank Accession No. M35021, Hunt et al., 1990, Gene 87:199-204; and hsp40: human: Genebank Accession No. D49547, Ohtsuka, 1993, Biochem. Biophys. Res. Commun. 197:235-240. Such sequences may be expressed using any appropriate expression vector known in the art. Suitable vectors include, but are not limited to, herpes simplex viral based vectors such as pHSV1 (Geller et al., 1990, Proc. Natl. Acad. Sci. U.S.A. 87:8950-8954); retroviral vectors such as MFG (Jaffee et al., 1993, Cancer Res. 53:2221-2226), and in particular Moloney retroviral vectors such as LN, LNSX, LNCX, and LXSN (Miller and Rosman, 1989, Biotechniques 7:980-989); vaccinia viral vectors such as MVA (Sutter and Moss, 1992, Proc. Natl. Acad. Sci. U.S.A. 89:10847-10851); adenovirus vectors such as pJM17 (Ali et al., 1994, Gene Therapy 1:367-384; Berker, 1988, Biotechniques 6:616-624; Wand and Finer, 1996, Nature Medicine 2:714-716); adeno-associated virus vectors such as AAV/neo (Mura-Cacho et al., 1992, J. Immunother. 11:231-237); pCDNA3 (InVitrogen); pET 11a, pET3a, pET11d, pET3d, pET22d, and pET12a (Novagen); plasmid AH5 (which contains the SV40 origin and the adenovirus major late promoter); pRC/CMV (InVitrogen); pCMU II (Paabo et al., 1986, EMBO J. 5:1921-1927); pZipNeo SV (Cepko et al., 1984, Cell 37:1053-1062) and pSRα (DNAX, Palo Alto, Calif.).

The affinity panning procedure may be varied in alternative embodiments of the present invention. For example, and as discussed more fully below, the binding buffer used to bind phage to hsp target, and/or the hsp target itself, may be modified chemically or by genetic engineering techniques.

In a first series of embodiments, a low ionic strength binding buffer, such as that used in the panning experiments of Blond-Elguini et al., 1993, Cell 75:717-728, may be used. A specific, nonlimiting example of such a binding buffer is 20 mM HEPES pH 7.5, 20 mM KCl, 10 mM (NH₄)₂SO₄, 2 mM MgCl₂, and 0.1-0.5% TWEEN 20. It should be noted that when a particular buffer such as HEPES or detergent such as TWEEN 20 is referred to, other species of buffer and/or detergent may be substituted by the skilled artisan.

In a second series of embodiments, a binding buffer having a higher ionic strength relative to the binding buffer of the foregoing paragraph may be used. Such higher ionic strength may more closely duplicate binding conditions between hsp target and peptide in vivo (i.e., be “physiologic”). In that regard, the ionic strength of the binding buffer, taking into consideration the buffer system and any salts present, may approximate the ionic strength of 100-150 mM NaCl. A nonlimiting example of a high ionic strength, or “physiologic,” buffer is 20 mM HEPES pH 7.5, 100 mM KCl, 1 mM MgAcetate, and 0.1-0.5% TWEEN 20.

In a third, related series of embodiments, a binding buffer which creates a molecular environment similar to that occurring at the native subcellular location of a hsp target may be used. For example, when the hsp target normally resides in the endoplasmic reticulum, the binding buffer may be designed to approximate the molecular conditions present in the endoplasmic reticulum. Because the endoplasmic reticulum contains an abundance of calcium ions, a binding buffer which comprises calcium ions (or one or more other species of divalent cation) may be used. In particular nonlimiting embodiments, the concentration of calcium ions may be 1-75 mM, preferably 1-50 mM, and more preferably 1-25 mM. Specific examples of such binding buffers include, but are not limited to: (i) 20 mM HEPES pH 7.5, 100 mM KCl, 25 mM CaCl₂, 5 mM MgAcetate, and 0.1-0.5% TWEEN 20; and (ii) 20 mM HEPES pH 7.5, 100 mM KCl, 1 mM CaAcetate, 1 mM MgAcetate and 0.1-0.5% TWEEN 20.

In a fourth series of embodiments, the binding buffer may comprise a reducing agent or an oxidizing agent. Suitable reducing agents include, but are not limited to, dithiothreitol (“DTT”), reduced glutathione, and beta mercaptoethanol; suitable oxidizing agents include, but are not limited to, oxidized glutathione. Specific nonlimiting examples of binding buffers which comprise a reducing agent include (i) 20 mM HEPES pH 7.5, 100 mM KCl, 1 mM CaCl₂, 1 mM DTT, 1 mM MgAcetate, and 0.1-0.5% TWEEN 20; and (ii) 20 mM HEPES pH 7.5, 100 mM KCl, 1 mM DTT, 1 mM MgAcetate, and 0.1-0.5% TWEEN 20.

In a fifth series of embodiments, the binding buffer may comprise a nucleotide which may, alternatively, be hydrolyzable or nonhydrolyzable. Such a binding buffer may be used to identify tethers which bind to a hsp target where the hsp target binds or releases peptides in association with nucleotide hydrolysis. For example, where the hsp target releases peptides in association with nucleotide hydrolysis, a non-hydrolyzable nucleotide may be comprised in the binding buffer. Suitable nucleotides include, but are not limited to, ATP, ADP, AMP, cAMP, AMP-PNP, GTP, GDP, GMP, etc.. Specific, nonlimiting examples of such binding buffers include (i) 20 mM HEPES pH 7.5, 100 mM KCl, 1 mM CaCl₂, 1 mM MgAcetate, 1 mM ATP (a hydrolyzable nucleotide) and 0.1-0.5% TWEEN 20; and (ii) 20 mM HEPES pH 7.5, 100 mM KCl, 1 mM CaCl₂, 1 mM MgAcetate, and 1 mM AMP-PNP (a non-hydrolyzable nucleotide).

The present invention also provides for methods of identifying tethers wherein the hsp target is a modified version of a naturally occurring heat shock protein, such that the hsp target provides a more efficient means for identifying tethers relative to the unmodified heat shock protein. For example, the conformation of a native heat shock protein may be altered to facilitate peptide binding; such a conformational change may be effected by binding the heat shock protein to one or more additional molecules to produce a hsp target. Such molecules may be other heat shock proteins or accessory molecules thereto. Alternatively, and particularly where peptides which bind to gp96 or hsp90 are sought, suitable molecules include members of the benzoquinone ansamycin antibiotics, such as herbimycin A, geldanamycin, macmimycin I, mimosamycin, and kuwaitimycin (Omura et al., 1979, J. Antibiotics 32:255-261), or structurally related compounds. In specific, nonlimiting examples, a 10-100 fold molar excess of a benzoquinone antibiotic relative to heat shock protein may be either combined with heat shock protein concurrent with adsorption onto a solid phase, or, alternatively, may be present during binding of phage. For example, a 50 fold molar excess of herbimycin A may be combined with gp96 or hsp90 concurrent with adsorption onto a solid substrate prior to affinity panning.

In related embodiments, the structure of a heat shock protein may be altered by truncation or by incorporation into a fusion protein to create a hsp target with enhanced peptide binding properties. For example, because a heat shock protein which normally acts in concert with other molecules may contain certain domains associated with binding those accessory molecules, and other domains which actually bind chaperoned peptides. The isolation of the latter for use as hsp target may provide a more efficient means of identifying suitable tethers. As a specific nonlimiting example, Wearsch and Nicchitta, 1996, Biochem. 35:16760-16769 have identified a C-terminal domain of grp94 which appears to be responsible for dimerization of that molecule; the removal of this domain from grp94 may produce a more efficient hsp target for identifying peptides that bind to grp94. Alternatively, the C-terminal domain alone may be used as an hsp target for identifying gp94 binding peptides, based on preliminary evidence that it has peptide binding capacity.

Phage-expressed peptides identified as binding to a hsp target using the above methods may then be sequenced and the contained peptides synthesized or recombinantly expressed in order to determine whether the expressed peptide itself binds to hsp target and may serve as an effective tether. Preferably, the same binding buffer used in affinity panning is used to evaluate peptide binding. A variety of techniques may be used to perform such an evaluation. For example, radiolabelled (e.g., iodine-125, carbon-14, or tritium-labeled) peptide may be exposed to hsp target under suitable conditions and labelled peptide/hsp target may then passed over a chromatographic resin such as Superdex 75, Superdex 200, Sepharose S300 or Superose 6; if binding has occurred, the labelled peptide and hsp target should co-migrate. Strength of binding may be evaluated by determining the conditions under which the association between the peptide and hsp target is broken. Peptides having various binding affinities to hsp target may be used in diverse clinical applications; it may be desirable to combine weakly antigenic peptides with strongly bound tethers. Alternatively, certain peptides may become tolerogenic when linked to a tether and bound to an hsp target and therefore it may be desirable to couple these antigenic peptides using weakly bound tethers.

### 5.2. CONJUGATE PEPTIDES

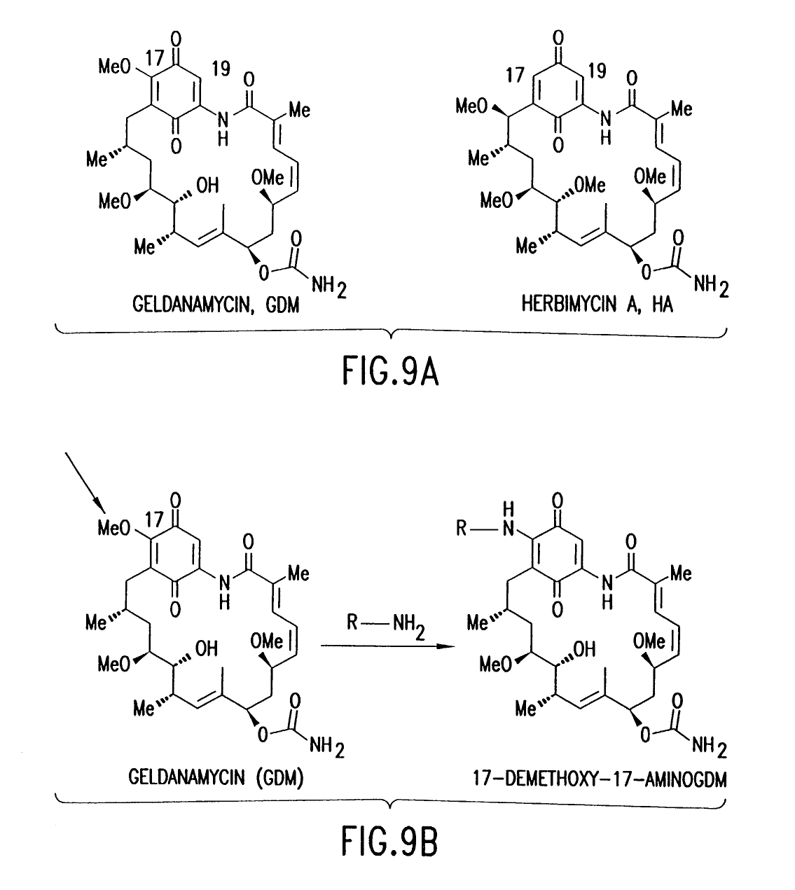

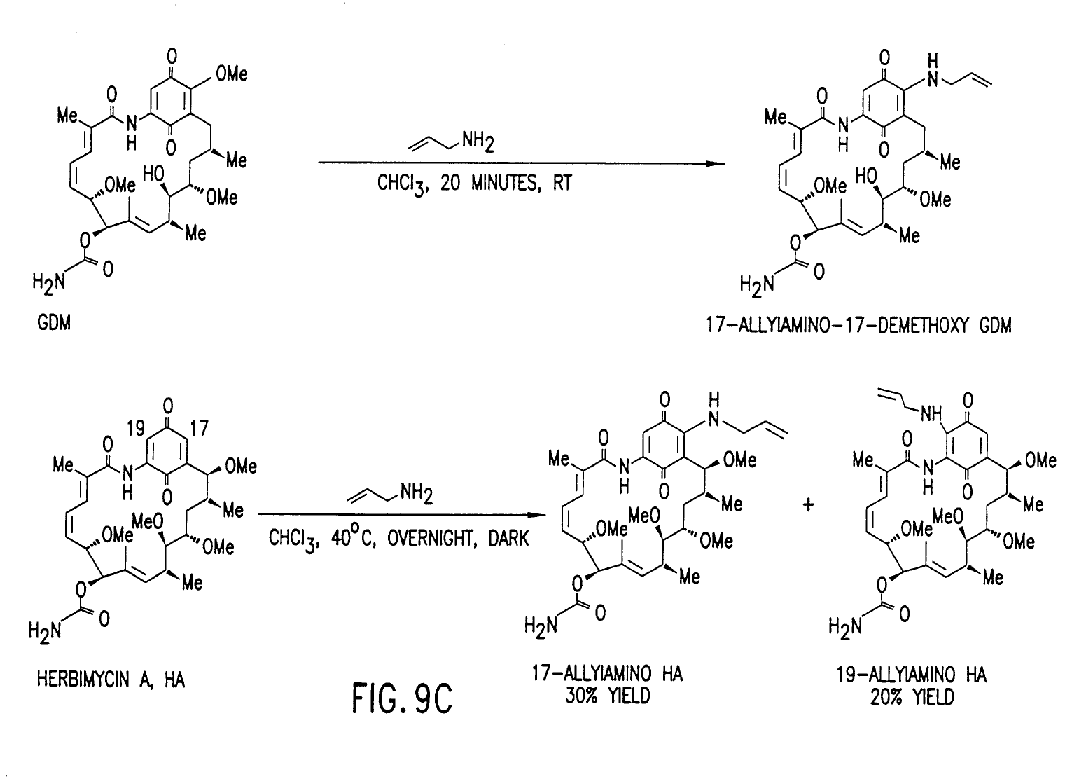

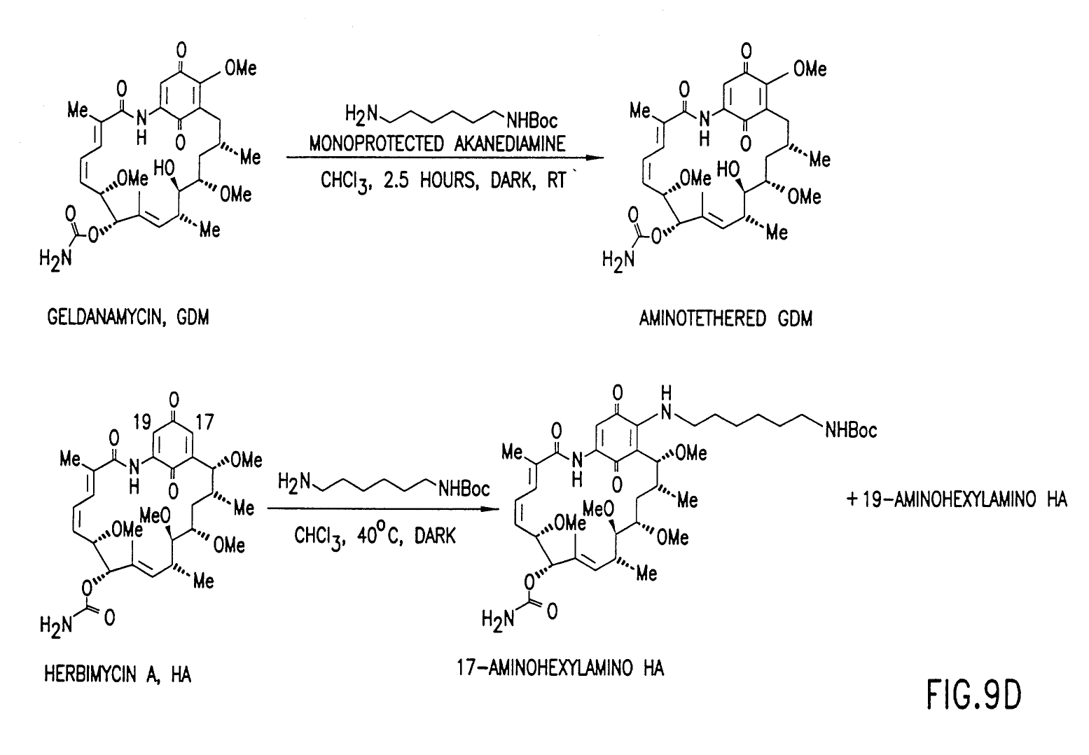

The present invention relates to conjugate peptides comprising (i) a portion which may be bound to a heat shock protein under physiologic conditions, referred to hereafter as the “tether”; and (ii) a portion which is antigenic (hereafter, the “antigenic peptide”). The term “peptide” as used herein refers to molecules which might otherwise be considered to be peptides or polypeptides within the art. The conjugate peptides of the invention may comprise portions which may or may not be peptides; such additional portions may improve stability, or target delivery, of the conjugate peptide. For example, in specific nonlimiting embodiments of the invention, the tether may comprise a benzoquinone ansamycin antibiotic such as geldanamycin or herbimycin A (see FIG. 9A); such tethers may or may not further comprise an hsp-binding peptide tether. The use of the term conjugate denotes that the conjugate peptides of the invention comprise an antigenic peptide covalently linked to another compound, which may or may not be another peptide, provided that the conjugate peptide is not found in nature. Thus, peptides which naturally bind to heat shock protein (and therefore contain an indigenous tether) and comprise an antigenic region are not “conjugate peptides” according to the invention. However, such naturally occurring peptides may be genetically engineered to position the indigenous tether in an altered position relative to the antigenic region, in which case a conjugate peptide according to the invention would be produced. In particular nonlimiting specific embodiments, the conjugate peptide may be an antigenic peptide from a natural source linked to a benzoquinone ansamycin antibiotic such as geldanamycin or herbimycin A; such a composition may or may not comprise additional peptide sequence.

The term “physiologic conditions”, as used herein, refers to conditions of temperature, pH, ionic strength, and molecular composition as are found within living organisms. For example, but not by way of limitation, physiological conditions would include temperatures of 4-55° C., and preferably 20-40° C.; a pH of 3-12, and preferably 5-8; and ionic strengths approximating the ionic strength of 50-300 mM NaCl, and preferably 100-200 mM NaCl. A specific, nonlimiting example of physiologic conditions includes phosphate buffered saline (13 mM NaH₂PO₄, 137 mM NaCl, pH 7.4) at 37° C. A conjugate peptide may bind to a heat shock protein under such conditions; however, a conjugate peptide also meets the definition set forth above if, having been bound to a heat shock protein under non-physiologic conditions, it remains bound under physiologic conditions, where, in preferred nonlimiting embodiments of the invention, said conjugate peptide/heat shock protein has a half-life of at least 1 minute, preferably at least 10 minutes, and more preferably 2-10 hours or longer.

The term “antigenic”, as used herein, refers to the capability of that portion of the conjugate peptide, either alone or in conjunction with either the tether or a heat shock protein or portion thereof, to elicit a cellular or humoral immune response in an organism or culture containing cells sensitized to respond to the corresponding antigen. An immune response is defined herein as a cellular or humoral immune response which is at least 2-fold greater, and preferably at least three-fold greater, than background levels.

Tethers which may be comprised in conjugate peptides of the invention may be identified using the methods set forth in the preceding section. Such tethers may have amino acid compositions which comprise a substantial proportion of hydrophobic amino acids such as phenylalanine and tryptophan, and/or a substantial number of serine, threonine, or proline residues. In particular, nonlimiting embodiments, tethers of the invention may comprise amino acid sequences which have the general description hydrophobic-basic-hydrophobic-hydrophobic-hydrophobic; Ser/Thr-hydrophobic-hydrophobic-Ser/Thr; Ser/Thr-Ser/Thr-hydrophobic-hydrophobic- Ser/Thr- Ser/Thr; and Ser/Thr-Ser/Thr-hydrophobic-hydrophobic-hydrophobic. Alternatively, tethers may comprise heat shock binding peptides as described in Blond-Elguindi et al., 1993, Cell 75:717-728, including the consensus sequence hydrophobic-(Trp/X)-hydrophobic-X-hydrophobic-X-hydrophobic and the specific peptides His Trp Asp Phe Ala Trp Pro Trp (SEQ ID NO: ) and Phe Trp Gly Leu Trp Pro Trp Glu (SEQ ID NO: ); Auger et al., 1996, Nature Med. 2:306-310, including Gln Lys Arg Ala Ala (SEQ ID NO: ) and Arg Arg Arg Ala Ala (SEQ ID NO: ); Flynn et al., 1989, Science 245:385-390; Gragerov et al., 1994, J. Mol. Biol. 235:848-854; Terlecky et al., 1992, J. Biol. Chem. 267:9202-9202, Lys Phe Glu Arg Gln (SEQ ID NO: ); and Nieland et al., 1996, Proc. Natl. Acad. Sci. U.S.A. 93:6135-6139, including the VSV8 peptide, Arg Gly Tyr Val Tyr Gln Gly Leu (SEQ ID NO: ). In preferred embodiments, tethers of the invention may have a length of 4-50 amino acid residues, and more preferably 7-20 amino acid residues.

In specific, nonlimiting embodiments, the following amino acid sequences, discussed more fully in the working examples which follow below, may be comprised, as tethers, in conjugate peptides according to the invention:

| | |
|---|---|
| Tyr Thr Leu Val Gln Pro Leu; | (SEQ ID NO: ) |
| Thr Pro Asp Ile Thr Pro Lys; | (SEQ ID NO: ) |
| Thr Tyr Pro Asp Leu Arg Tyr; | (SEQ ID NO: ) |
| Asp Arg Thr His Ala Thr Ser; | (SEQ ID NO: ) |
| Met Ser Thr Thr Phe Tyr Ser; | (SEQ ID NO: ) |
| Tyr Gln His Ala Val Gln Thr; | (SEQ ID NO: ) |
| Phe Pro Phe Ser Ala Ser Thr; | (SEQ ID NO: ) |
| Ser Ser Phe Pro Pro Leu Asp; | (SEQ ID NO: ) |
| Met Ala Pro Ser Pro Pro His; | (SEQ ID NO: ) |
| Ser Ser Phe Pro Asp Leu Leu; | (SEQ ID NO: ) |
| His Ser Tyr Asn Arg Leu Pro; | (SEQ ID NO: ) |
| His Leu Thr His Ser Gln Arg; | (SEQ ID NO: ) |
| Gln Ala Ala Gln Ser Arg Ser; | (SEQ ID NO: ) |
| Phe Ala Thr His His Ile Gly; | (SEQ ID NO: ) |
| Ser Met Pro Glu Pro Leu Ile; | (SEQ ID NO: ) |
| Ile Pro Arg Tyr His Leu Ile; | (SEQ ID NO: ) |
| Ser Ala Pro His Met Thr Ser; | (SEQ ID NO: ) |
| Lys Ala Pro Val Trp Ala Ser; | (SEQ ID NO: ) |
| Leu Pro His Trp Leu Leu Ile; | (SEQ ID NO: ) |
| Ala Ser Ala Gly Tyr Gln Ile; | (SEQ ID NO: ) |
| Val Thr Pro Lys Thr Gly Ser; | (SEQ ID NO: ) |
| Glu His Pro Met Pro Val Leu; | (SEQ ID NO: ) |
| Val Ser Ser Phe Val Thr Ser; | (SEQ ID NO: ) |
| Ser Thr His Phe Thr Trp Pro; | (SEQ ID NO: ) |
| Gly Gln Trp Trp Ser Pro Asp; | (SEQ ID NO: ) |
| Gly Pro Pro His Gln Asp Ser; | (SEQ ID NO: ) |
| Asn Thr Leu Pro Ser Thr Ile; | (SEQ ID NO: ) |
| His Gln Pro Ser Arg Trp Val; | (SEQ ID NO: ) |
| Tyr Gly Asn Pro Leu Gln Pro; | (SEQ ID NO: ) |
| Phe His Trp Trp Trp Gln Pro; | (SEQ ID NO: ) |
| Ile Thr Leu Lys Tyr Pro Leu; | (SEQ ID NO: ) |
| Phe His Trp Pro Trp Leu Phe; | (SEQ ID NO: ) |
| Thr Ala Gln Asp Ser Thr Gly; | (SEQ ID NO: ) |
| Phe His Trp Trp Trp Gln Pro; | (SEQ ID NO: ) |
| Phe His Trp Trp Asp Trp Trp; | (SEQ ID NO: ) |
| Glu Pro Phe Phe Arg Met Gln; | (SEQ ID NO: ) |
| Thr Trp Trp Leu Asn Tyr Arg; | (SEQ ID NO: ) |
| Phe His Trp Trp Trp Gln Pro; | (SEQ ID NO: ) |
| Gln Pro Ser His Leu Arg Trp; | (SEQ ID NO: ) |
| Ser Pro Ala Ser Pro Val Tyr; | (SEQ ID NO: ) |
| Phe His Trp Trp Trp Gln Pro; | (SEQ ID NO: ) |
| His Pro Ser Asn Gln Ala Ser; | (SEQ ID NO: ) |
| Asn Ser Ala Pro Arg Pro Val; | (SEQ ID NO: ) |
| Gln Leu Trp Ser Ile Tyr Pro; | (SEQ ID NO: ) |
| Ser Trp Pro Phe Phe Asp Leu; | (SEQ ID NO: ) |
| Asp Thr Thr Leu Pro Leu His; | (SEQ ID NO: ) |
| Trp His Trp Gln Met Leu Trp; | (SEQ ID NO: ) |
| Asp Ser Phe Arg Thr Pro Val; | (SEQ ID NO: ) |
| Thr Ser Pro Leu Ser Leu Leu; | (SEQ ID NO: ) |
| Ala Tyr Asn Tyr Val Ser Asp; | (SEQ ID NO: ) |
| Arg Pro Leu His Asp Pro Met; | (SEQ ID NO: ) |
| Trp Pro Ser Thr Thr Leu Phe; | (SEQ ID NO: ) |
| Ala Thr Leu Glu Pro Val Arg; | (SEQ ID NO: ) |
| Ser Met Thr Val Leu Arg Pro; | (SEQ ID NO: ) |
| Gln Ile Gly Ala Pro Ser Trp; | (SEQ ID NO: ) |
| Ala Pro Asp Leu Tyr Val Pro; | (SEQ ID NO: ) |
| Arg Met Pro Pro Leu Leu Pro; | (SEQ ID NO: ) |
| Ala Lys Ala Thr Pro Glu His; | (SEQ ID NO: ) |
| Thr Pro Pro Leu Arg Ile Asn; | (SEQ ID NO: ) |
| Leu Pro Ile His Ala Pro His; | (SEQ ID NO: ) |
| Asp Leu Asn Ala Tyr Thr His; | (SEQ ID NO: ) |
| Val Thr Leu Pro Asn Phe His; | (SEQ ID NO: ) |
| Asn Ser Arg Leu Pro Thr Leu; | (SEQ ID NO: ) |
| Tyr Pro His Pro Ser Arg Ser; | (SEQ ID NO: ) |
| Gly Thr Ala His Phe Met Tyr; | (SEQ ID NO: ) |
| Tyr Ser Leu Leu Pro Thr Arg; | (SEQ ID NO: ) |
| Leu Pro Arg Arg Thr Leu Leu; | (SEQ ID NO: ) |
| Thr Ser Thr Leu Leu Trp Lys; | (SEQ ID NO: ) |
| Thr Ser Asp Met Lys Pro His; | (SEQ ID NO: ) |
| Thr Ser Ser Tyr Leu Ala Leu; | (SEQ ID NO: ) |
| Asn Leu Tyr Gly Pro His Asp; | (SEQ ID NO: ) |
| Leu Gln Thr Tyr Thr Ala Ser; | (SEQ ID NO: ) |
| Ala Tyr Lys Ser Leu Thr Gln; | (SEQ ID NO: ) |
| Ser Thr Ser Val Tyr Ser Ser; | (SEQ ID NO: ) |
| Glu Gly Pro Leu Arg Ser Pro; | (SEQ ID NO: ) |
| Thr Thr Tyr His Ala Leu Gly; | (SEQ ID NO: ) |
| Val Ser Ile Gly His Pro Ser; | (SEQ ID NO: ) |
| Thr His Ser His Arg Pro Ser; | (SEQ ID NO: ) |
| Ile Thr Asn Pro Leu Thr Thr; | (SEQ ID NO: ) |
| Ser Ile Gln Ala His His Ser; | (SEQ ID NO: ) |
| Leu Asn Trp Pro Arg Val Leu; | (SEQ ID NO: ) |
| Tyr Tyr Tyr Ala Pro Pro Pro; | (SEQ ID NO: ) |
| Ser Leu Trp Thr Arg Leu Pro; | (SEQ ID NO: ) |
| Asn Val Tyr His Ser Ser Leu; | (SEQ ID NO: ) |
| Asn Ser Pro His Pro Pro Thr; | (SEQ ID NO: ) |
| Val Pro Ala Lys Pro Arg His; | (SEQ ID NO: ) |
| His Asn Leu His Pro Asn Arg; | (SEQ ID NO: ) |
| Tyr Thr Thr His Arg Trp Leu; | (SEQ ID NO: ) |
| Ala Val Thr Ala Ala Ile Val; | (SEQ ID NO: ) |
| Thr Leu Met His Asp Arg Val; | (SEQ ID NO: ) |
| Thr Pro Leu Lys Val Pro Tyr; | (SEQ ID NO: ) |
| Phe Thr Asn Gln Gln Tyr His; | (SEQ ID NO: ) |
| Ser His Val Pro Ser Met Ala; | (SEQ ID NO: ) |
| His Thr Thr Val Tyr Gly Ala; | (SEQ ID NO: ) |
| Thr Glu Thr Pro Tyr Pro Thr; | (SEQ ID NO: ) |
| Leu Thr Thr Pro Phe Ser Ser; | (SEQ ID NO: ) |
| Gly Val Pro Leu Thr Met Asp; | (SEQ ID NO: ) |
| Lys Leu Pro Thr Val Leu Arg; | (SEQ ID NO: ) |
| Cys Arg Phe His Gly Asn Arg; | (SEQ ID NO: ) |
| Tyr Thr Arg Asp Phe Glu Ala; | (SEQ ID NO: ) |
| Ser Ser Ala Ala Gly Pro Arg; | (SEQ ID NO: ) |
| Ser Leu Ile Gln Tyr Ser Arg; | (SEQ ID NO: ) |
| Asp Ala Leu Met Trp Pro UKN; | (SEQ ID NO: ) |
| Ser Ser UKN Ser Leu Tyr Ile; | (SEQ ID NO: ) |
| Phe Asn Thr Ser Thr Arg Thr; | (SEQ ID NO: ) |
| Thr Val Gln His Val Ala Phe; | (SEQ ID NO: ) |
| Asp Tyr Ser Phe Pro Pro Leu; | (SEQ ID NO: ) |
| Val Gly Ser Met Glu Ser Leu; | (SEQ ID NO: ) |
| Phe UKN Pro Met Ile UKN Ser; | (SEQ ID NO: ) |
| Ala Pro Pro Arg Val Thr Met; | (SEQ ID NO: ) |
| Ile Ala Thr Lys Thr Pro Lys; | (SEQ ID NO: ) |
| Lys Pro Pro Leu Phe Gln Ile; | (SEQ ID NO: ) |
| Tyr His Thr Ala His Asn Met; | (SEQ ID NO: ) |
| Ser Tyr Ile Gln Ala Thr His; | (SEQ ID NO: ) |
| Ser Ser Phe Ala Thr Phe Leu; | (SEQ ID NO: ) |
| Thr Thr Pro Pro Asn Phe Ala; | (SEQ ID NO: ) |
| Ile Ser Leu Asp Pro Arg Met; | (SEQ ID NO: ) |
| Ser Leu Pro Leu Phe Gly Ala; | (SEQ ID NO: ) |
| Asn Leu Leu Lys Thr Thr Leu; | (SEQ ID NO: ) |
| Asp Gln Asn Leu Pro Arg Arg; | (SEQ ID NO: ) |
| Ser His Phe Glu Gln Leu Leu; | (SEQ ID NO: ) |
| Thr Pro Gln Leu His His Gly; | (SEQ ID NO: ) |
| Ala Pro Leu Asp Arg Ile Thr; | (SEQ ID NO: ) |
| Phe Ala Pro Leu Ile Ala His; | (SEQ ID NO: ) |
| Ser Trp Ile Gln Thr Phe Met; | (SEQ ID NO: ) |
| Asn Thr Trp Pro His Met Tyr; | (SEQ ID NO: ) |
| Glu Pro Leu Pro Thr Thr Leu; | (SEQ ID NO: ) |
| His Gly Pro His Leu Phe Asn; | (SEQ ID NO: ) |
| Tyr Leu Asn Ser Thr Leu Ala; | (SEQ ID NO: ) |
| His Leu His Ser Pro Ser Gly; | (SEQ ID NO: ) |
| Thr Leu Pro His Arg Leu Asn; | (SEQ ID NO: ) |
| Ser Ser Pro Arg Glu Val His; | (SEQ ID NO: ) |
| Asn Gln Val Asp Thr Ala Arg; | (SEQ ID NO: ) |
| Tyr Pro Thr Pro Leu Leu Thr; | (SEQ ID NO: ) |
| His Pro Ala Ala Phe Pro Trp; | (SEQ ID NO: ) |
| Leu Leu Pro His Ser Ser Ala; | (SEQ ID NO: ) |
| Leu Glu Thr Tyr Thr Ala Ser; | (SEQ ID NO: ) |
| Lys Tyr Val Pro Leu Pro Pro; | (SEQ ID NO: ) |
| Ala Pro Leu Ala Leu His Ala; | (SEQ ID NO: ) |
| Tyr Glu Ser Leu Leu Thr Lys; | (SEQ ID NO: ) |
| Ser His Ala Ala Ser Gly Thr; | (SEQ ID NO: ) |
| Gly Leu Ala Thr Val Lys Ser; | (SEQ ID NO: ) |
| Gly Ala Thr Ser Phe Gly Leu; | (SEQ ID NO: ) |
| Lys Pro Pro Gly Pro Val Ser; | (SEQ ID NO: ) |
| Thr Leu Tyr Val Ser Gly Asn; | (SEQ ID NO: ) |
| His Ala Pro Phe Lys Ser Gln; | (SEQ ID NO: ) |
| Val Ala Phe Thr Arg Leu Pro; | (SEQ ID NO: ) |
| Leu Pro Thr Arg Thr Pro Ala; | (SEQ ID NO: ) |
| Ala Ser Phe Asp Leu Leu Ile; | (SEQ ID NO: ) |
| Arg Met Asn Thr Glu Pro Pro; | (SEQ ID NO: ) |
| Lys Met Thr Pro Leu Thr Thr; | (SEQ ID NO: ) |
| Ala Asn Ala Thr Pro Leu Leu; | (SEQ ID NO: ) |
| Thr Ile Trp Pro Pro Pro Val; | (SEQ ID NO: ) |
| Gln Thr Lys Val Met Thr Thr; | (SEQ ID NO: ) |
| Asn His Ala Val Phe Ala Ser; | (SEQ ID NO: ) |
| Leu His Ala Ala UKN Thr Ser; | (SEQ ID NO: ) |
| Thr Trp Gln Pro Tyr Phe His; | (SEQ ID NO: ) |
| Ala Pro Leu Ala Leu His Ala; | (SEQ ID NO: ) |
| Thr Ala His Asp Leu Thr Val; | (SEQ ID NO: ) |
| Asn Met Thr Asn Met Leu Thr; | (SEQ ID NO: ) |
| Gly Ser Gly Leu Ser Gln Asp; | (SEQ ID NO: ) |
| Thr Pro Ile Lys Thr Ile Tyr; | (SEQ ID NO: ) |
| Ser His Leu Tyr Arg Ser Ser; and | (SEQ ID NO: ) |
| His Gly Gln Ala Trp Gln Phe. | (SEQ ID NO: ) |

(UKN indicates that the species of amino acid at
that residue is not known).

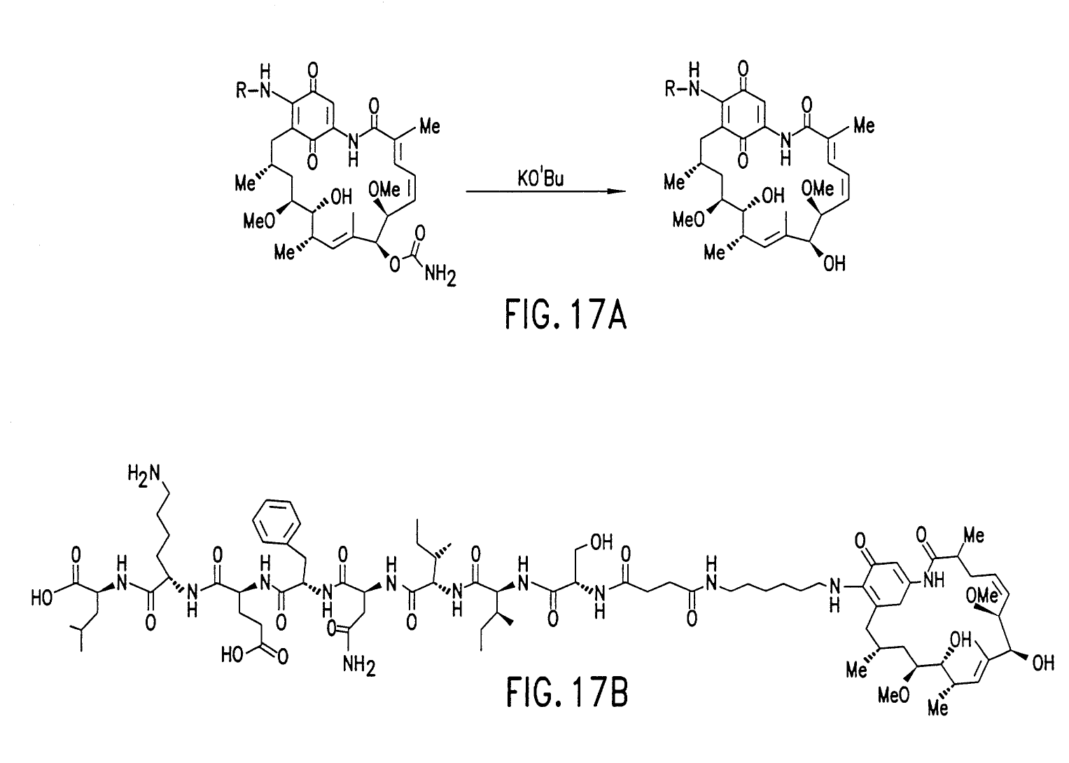

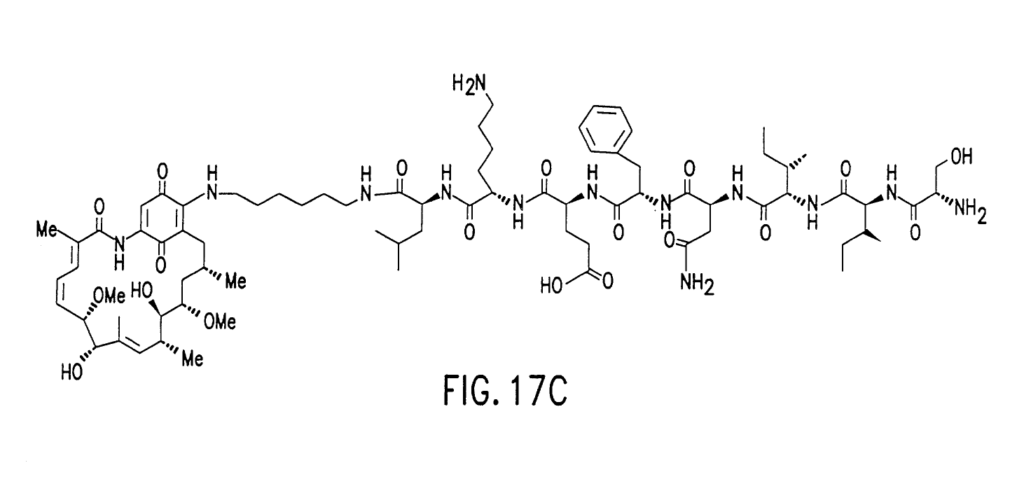

In a series of nonlimiting embodiments, conjugate peptides of the invention may comprise a benzoquinone ansamycin antibiotic molecule and an antigenic peptide. Such conjugate peptides may be produced by covalently linking a benzoquinone ansamycin antibiotic to an antigenic peptide. Suitable benzoquinone ansamycin antibiotics include, but are not limited to, herbimycin A, geldanamycin, mimosamycin, macmimycin I and kuwaitimycin, as well as analogs and derivatives thereof. In nonlimiting embodiments, it may be desirable to utilize a benzoquinone ansamycin antibiotic having greater or lesser affinity for heat shock protein relative to herbimycin A or geldanamycin: a specific nonlimiting example of such a compound is 8-decarbamoyl geldanamycin, which has a lower affinity for heat shock protein, and which may be produced by reacting geldanamycin with potassium tertbutyloxide in dimethylformamide (see FIG. 17).

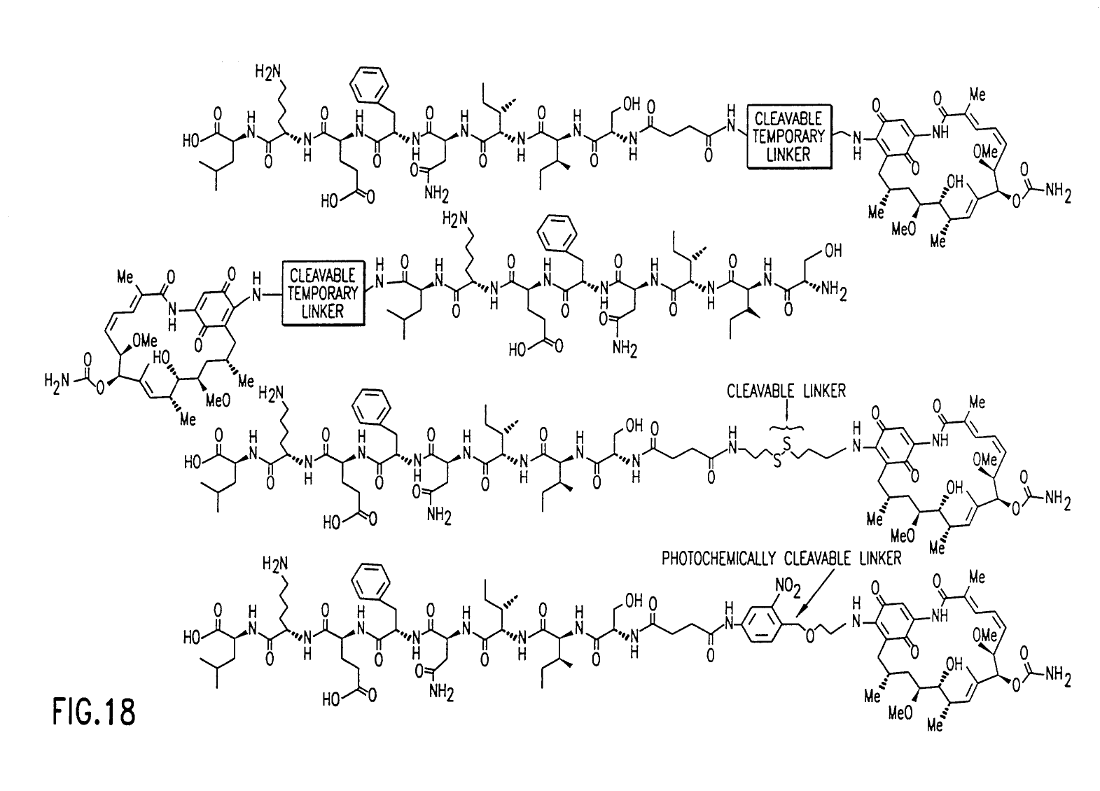

A chemical structure which, if present, connects benzoquinone ansamycin antibiotic and antigenic peptide is referred to herein as a “linker”.The linker may or, alternatively, may not be a peptide, or may comprise both peptide as well as non-peptide components. The linker may be designed to provide an optimized association between the conjugate peptide and a heat shock protein. Features of a linker which may be relevant in this regard include not only its length, but also its polarity, hydrophobicity (for example, as provided by aliphatic or aromatic side chains), heteroatom composition (e.g., the presence of ethers and/or amines (primary, secondary, or tertiary)) the presence of sulfur derivatives (e.g., sulfides, sulfoxides and sulfones) and/or phosphorous derivatives (e.g., phosphines, phosphites, phosphinates, and phosphates) and the like. In specific, nonlimiting examples of the invention, a cleavable linker, for example, a linker which is acid sensitive, base sensitive, light sensitive, sensitive to reduction or oxidation or to cleavage by a cellular enzyme may be used (see FIG. 18).

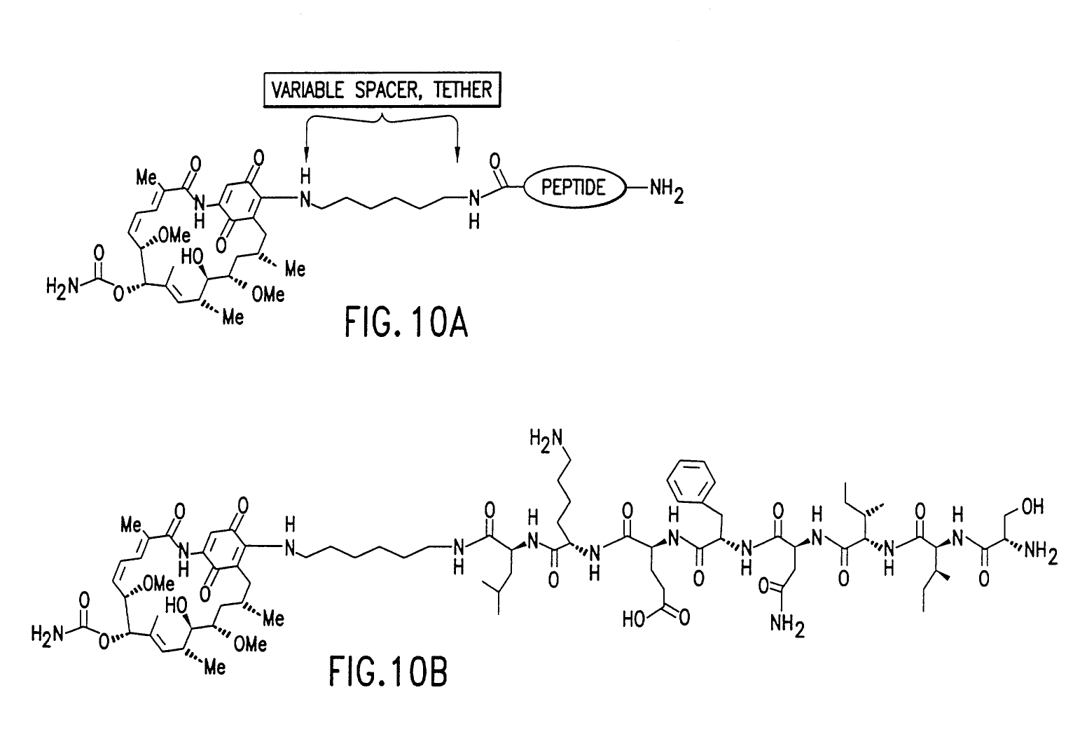

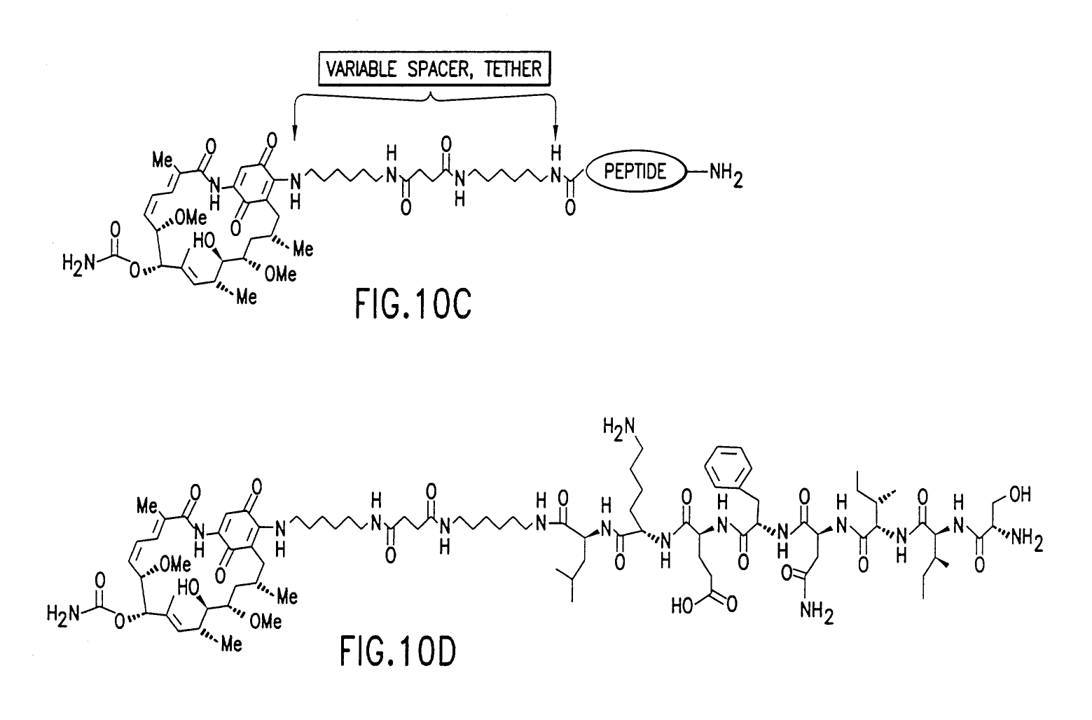

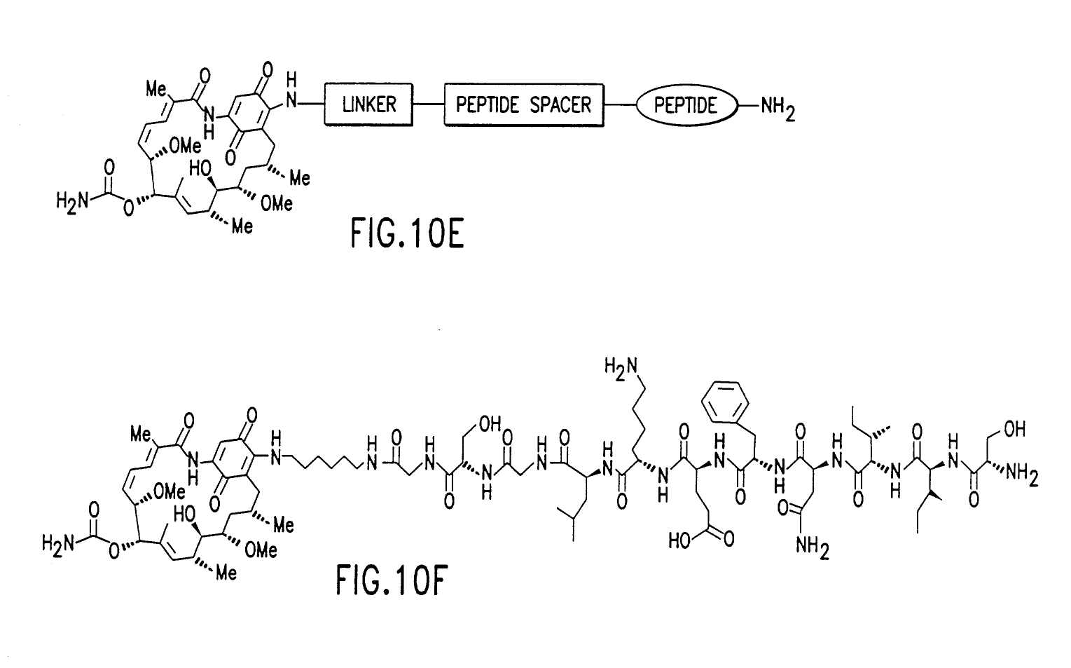

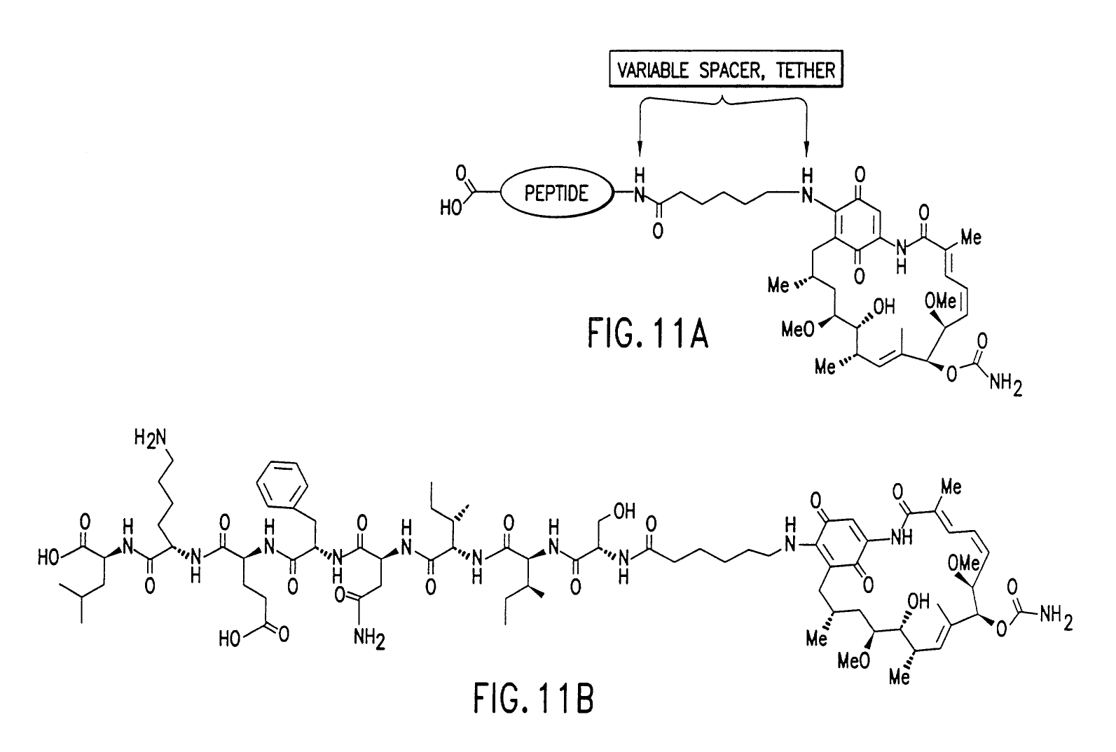

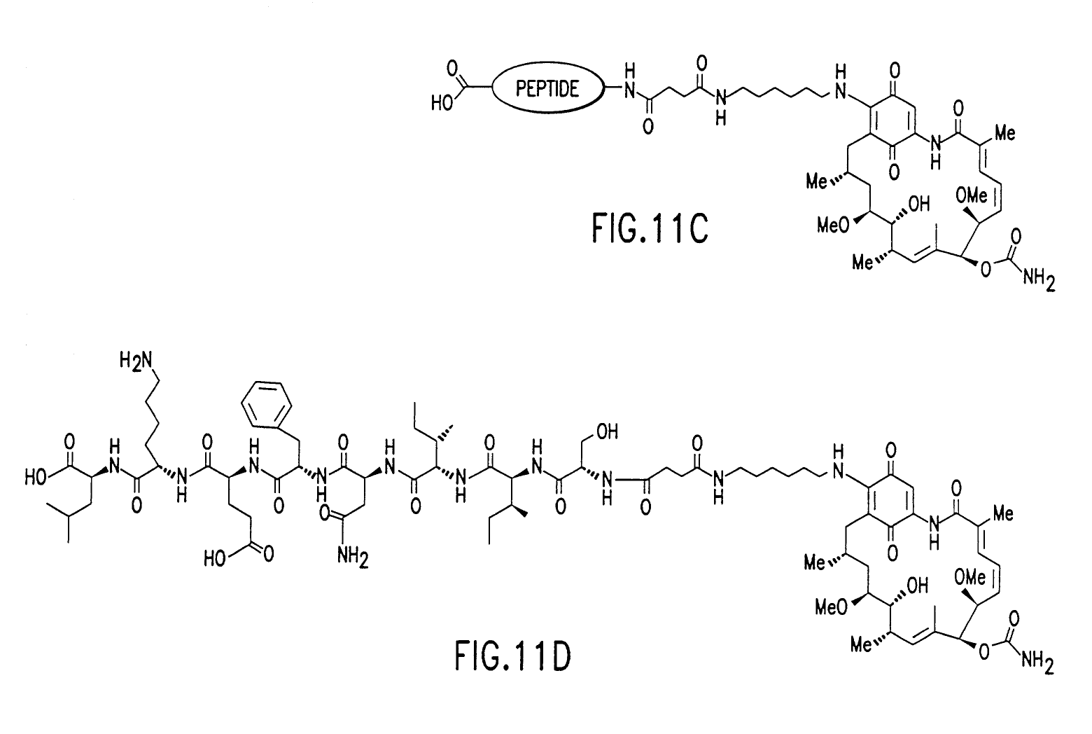

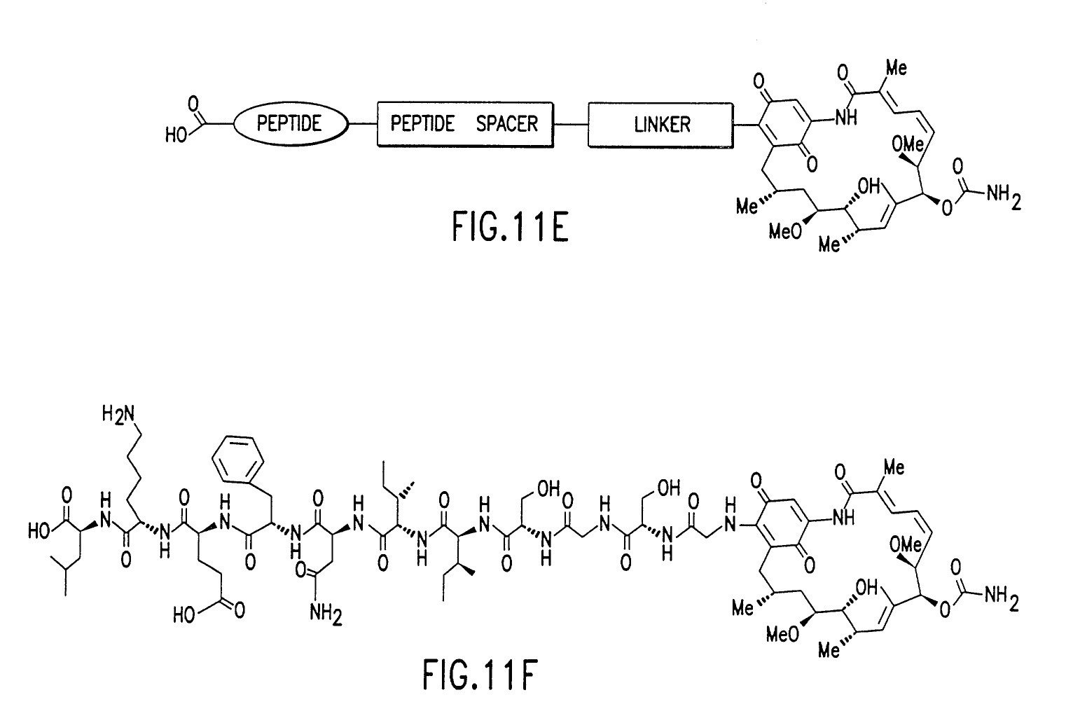

A peptide comprising an antigenic peptide may be covalently bound to the benzoquinone ansamycin antibiotic by either its amino or carboxyl terminus or via reactive side chains. The binding affinity of the resulting conjugate peptides for heat shock protein may be evaluated in order to select the optimal linkage site. FIG. 10A-F depict antigenic peptides covalently bound to a benzoquinone ansamycin antibiotic (geldanamycin is shown in the figure) via the peptide's carboxyl terminus. Alternatively, the benzoquinone ansamycin antibiotic may be covalently bound to the amino terminus of the peptide, as shown in FIG. 11A-F.

In a specific, nonlimiting embodiment of the invention, conjugate peptides comprising benzoquinone ansamycin antibiotics may be prepared according to the following scheme. In view of the X-ray structure of the site of interaction between geldanamycin and hsp90, it may be desirable to link geldanamycin or herbimycin A to antigenic peptide at carbon 17 of these antibiotics. Primary amines appear to react readily with geldanamycin at this position to produce 17-demethoxy-17 alkyl amino geldanamycin, as shown in FIG. 9B. Although the reactivity of herbimycin A is quite similar to that of geldanamycin, the reaction of allyl amine with geldanamycin gives rise to a single compound, 17-allylamino-17-demethoxygeldanamycin, whereas allylamine reacts with herbimycin A at a higher temperature and for a longer reaction time to produce two derivatives, namely 17-allylamino herbimycin and 19-allylamino herbimyicn, in a ratio of approximately 3 to 2, respectively (FIG. 9C). 17-allylamino herbimycin is more active than 19-allylamino herbimycin, which is consistent with the X-ray diffraction pattern of geldanamycin/hsp90 (Stebbins et al., 1997, Cell 89:239-250).

Because herbimycin A is less reactive than geldanamnycin towards amine nucleophiles, it is desirable to form a linker between herbimycin A and antigenic peptide as follows. Herbimycin A may be reacted with a monoprotected alkanediamine in chloroform, at 40-60° C. for 8-24 hours in the dark to produce a mixture of the 17 and 19-monoprotected alkanediamino herbimycin. These two compounds may then be separated by chromatography, and the desired 17-derivative collected, deprotected and then submitted to the same conditions used to prepare antigenic peptide linked to geldanamycin (see FIG. 9D).

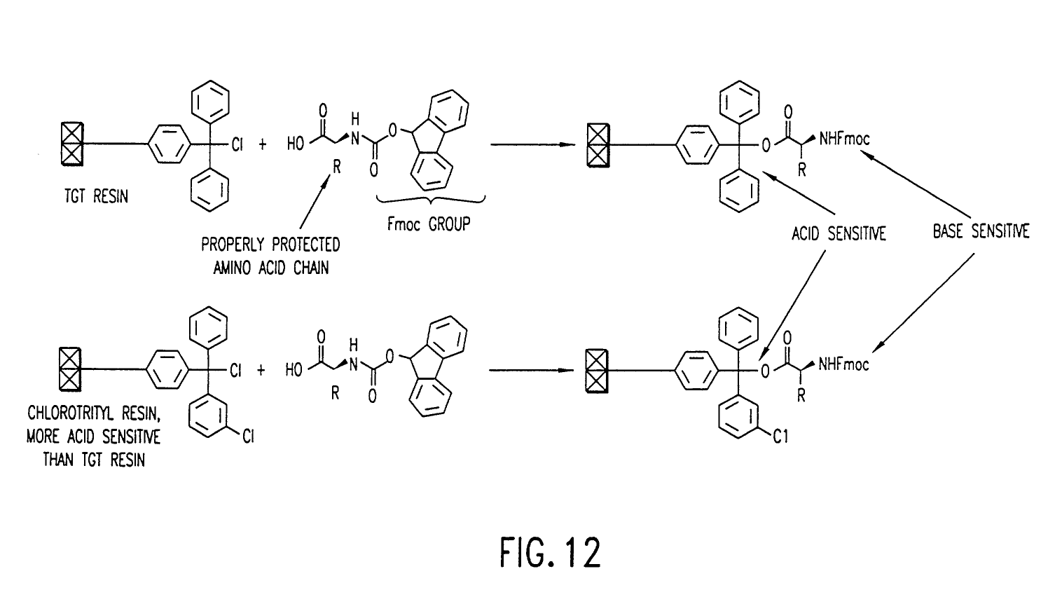

For the preparation of a conjugate peptide comprising a benzoquinone ansamycin antibiotic, a synthetic scheme may be utilized such that both the amino end and the carboxyl end of the antigenic peptide may be functionalized using the same protected peptide precursor; in other words, the same protected peptide may be used in the preparation of either amino-linked or carboxyl-linked conjugate peptides. For example, the peptide may be prepared on a solid support, such as a resin, to improve efficiency. In choosing a resin, it should be considered that at the end of the synthesis, in order to prepare carboxyl-linked conjugate peptides the carboxylic acid group should be selectively hydrolyzed so that the peptide is released from the resin without deprotecting any amino acid in the peptide (Bollhagen et al., 1994, J. Chem. Soc., Chem. Corn. 2559; Coste et al., 1990, Tetrahed. Let. 31:205; Rovero et al., 1993, Tetrahed. Let. 34:2199; Carpino and El-Faham, 1995, J. Org. Chem. 60:3561; Sieber and Riniker, 1991, Tetrahed. Let. 32:739; Dolling et al., 1994, J. Chem. Soc. Chem. Commun. 853; Lapatsanis et al., J. Chem. Soc. Chem. Commun. 671; Barlos et al., 1991, Int. J. Peptide Protein Res. 37:513; Houghten et al., 1986, Int. J. Peptide Protein Res. 27:653; Riniker et al., 1993, Tetrahed. 49:9307). This also ensures that the sequence does not contain any contamination or impurities that often result from the reaction of peripheral functionalities on the peptide chain. As specific, nonlimiting examples, NovaBiochem TGT or ClTrt resins may be used (see FIG. 12); these are polymeric resins with trityl or chlorotrityl end protecting groups, respectively. Where a TGT resin is used, the first amino acid is attached to the resin as an acid sensitive trityl ester. In fact, this functionality is very sensitive even to mild acids, thereby enhancing the selectivity in the eventual deprotection of the peptide. An analogous procedure may be applied using Cltrt resin. It should further be noted that the protecting groups on the peptide chain are desirably compatible with the coupling and deprotection conditions that are applied throughout the synthesis of the peptide.

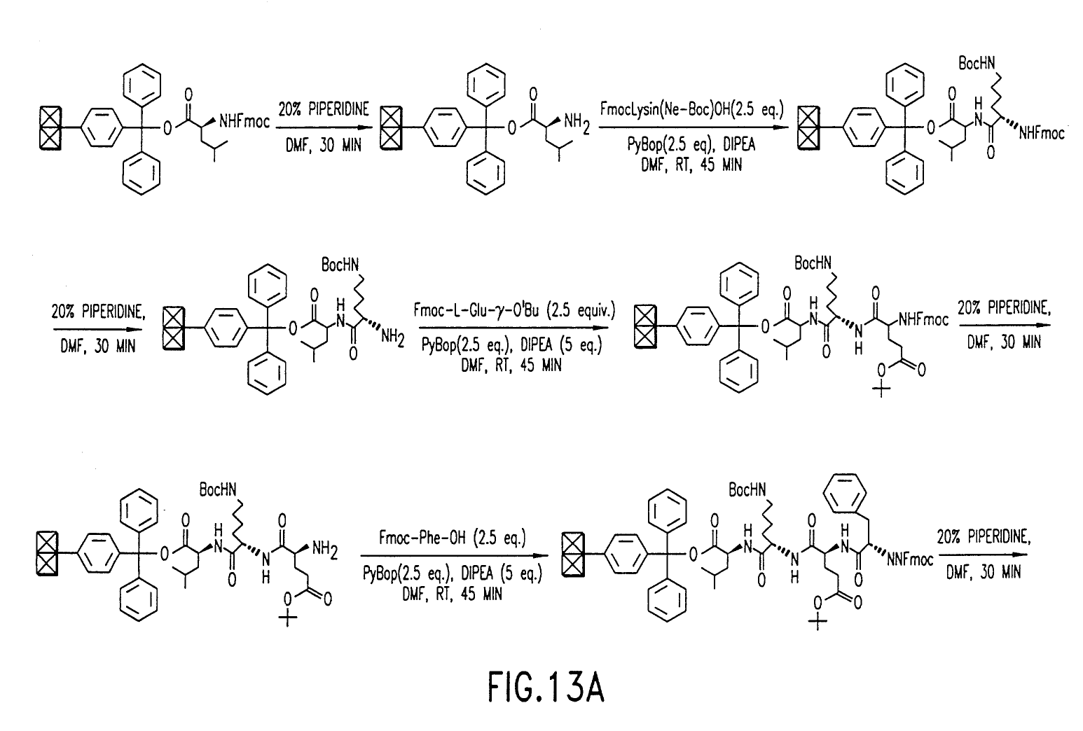

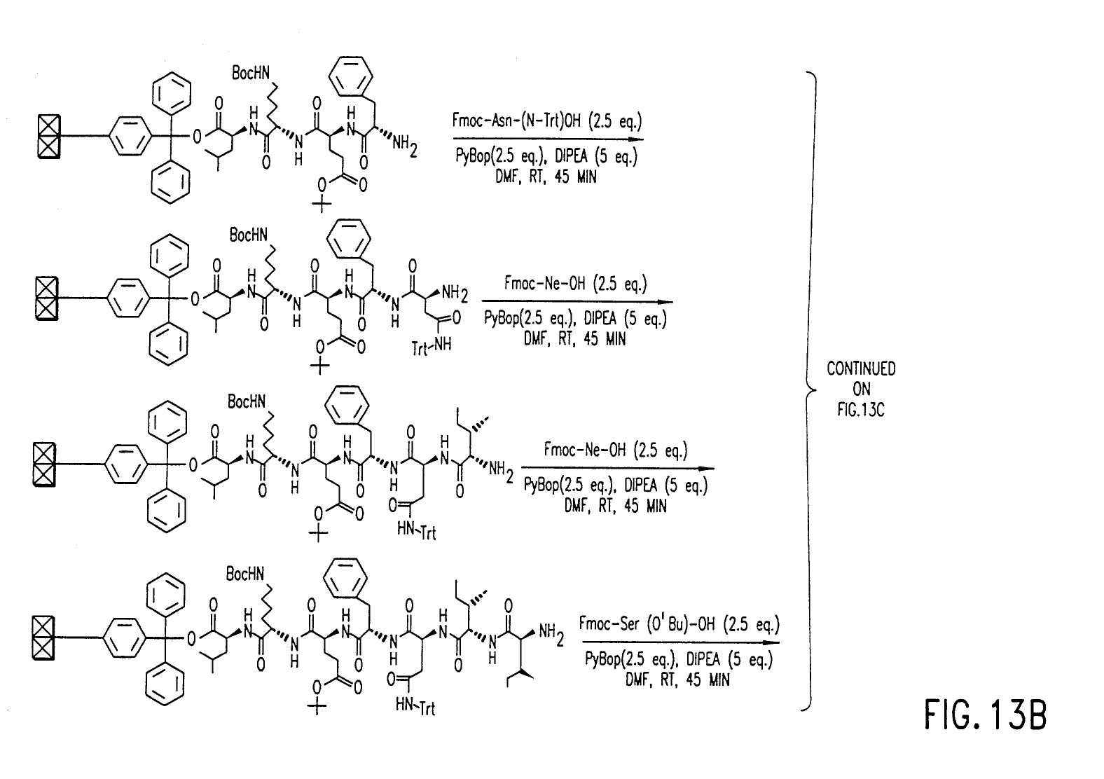

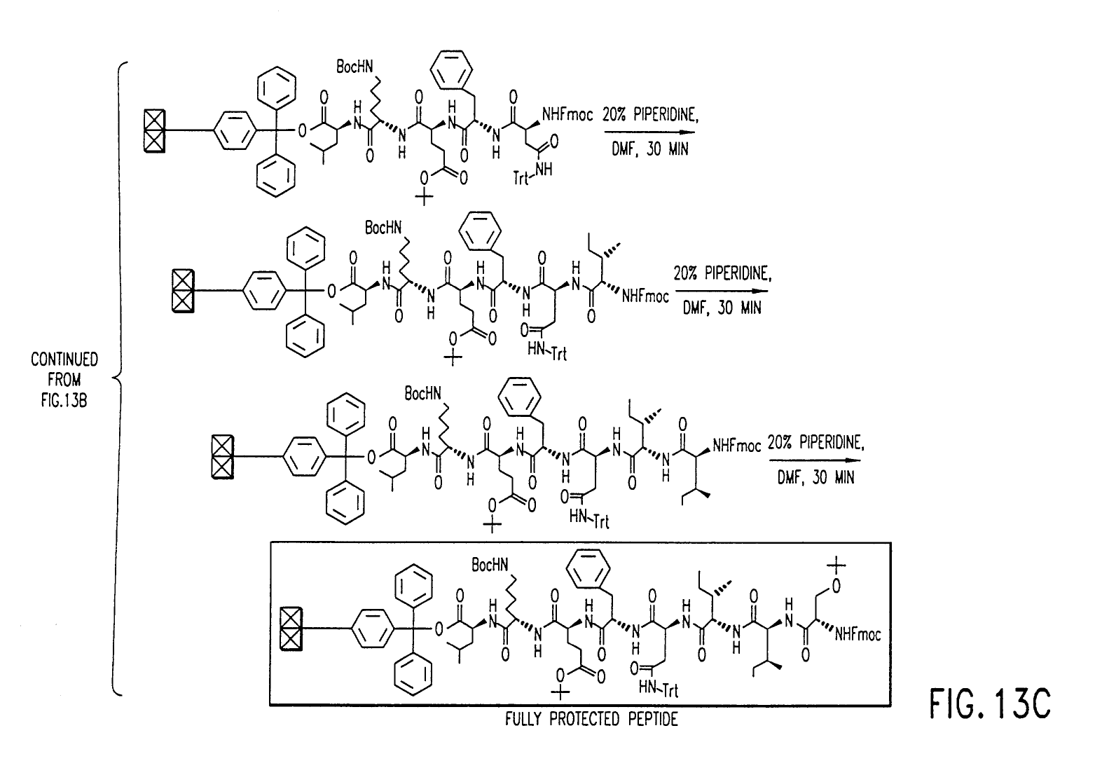

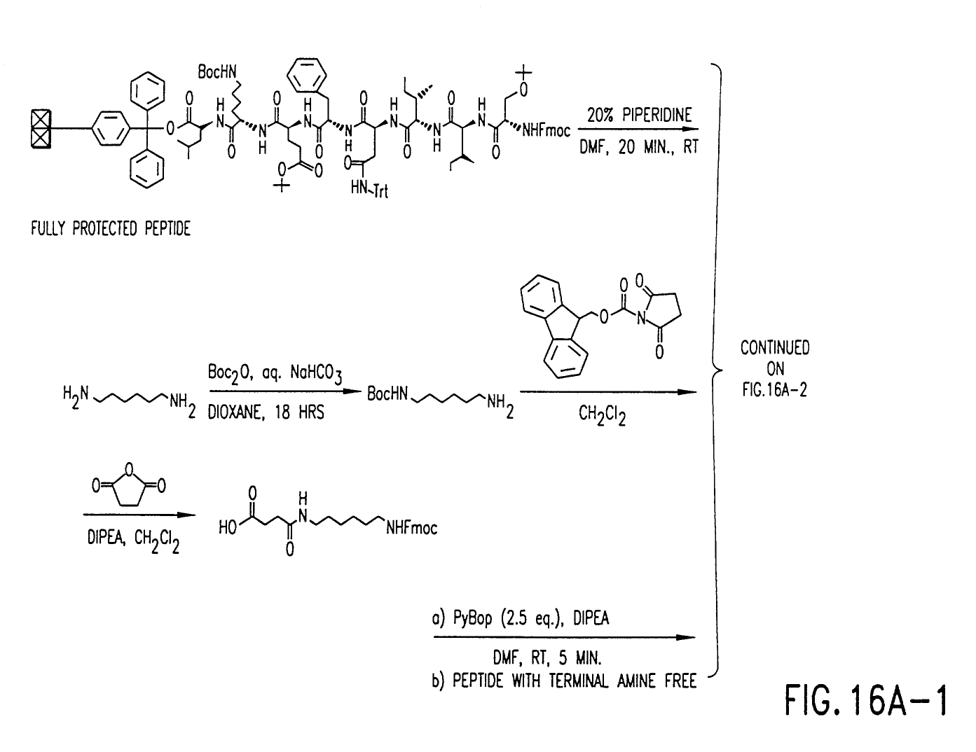

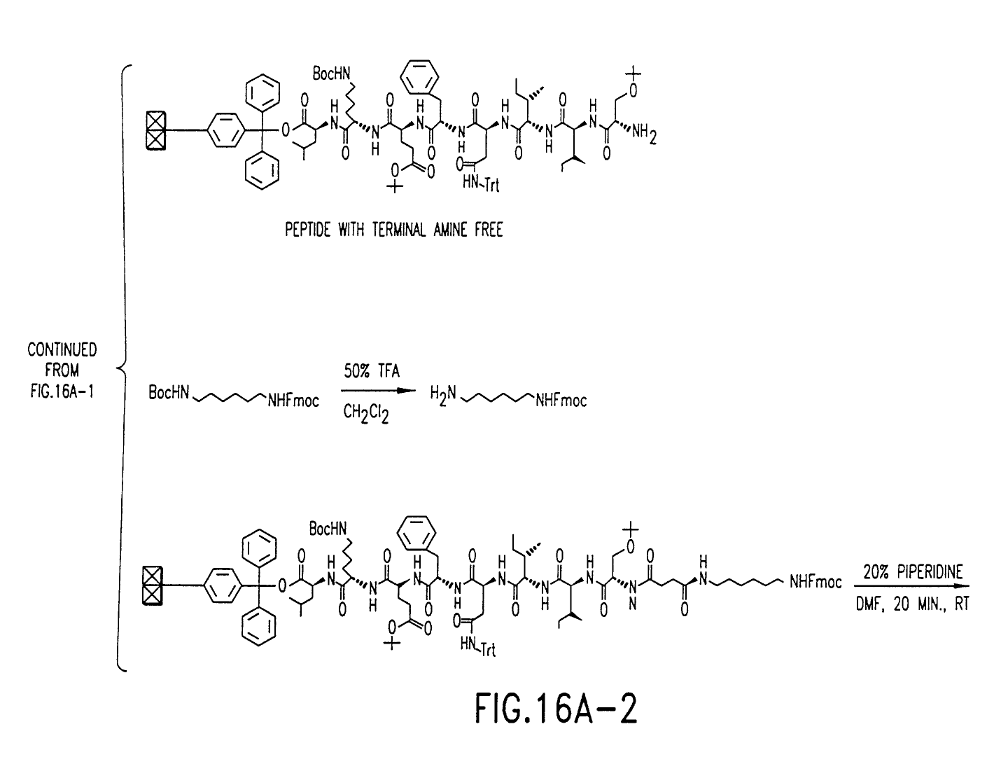

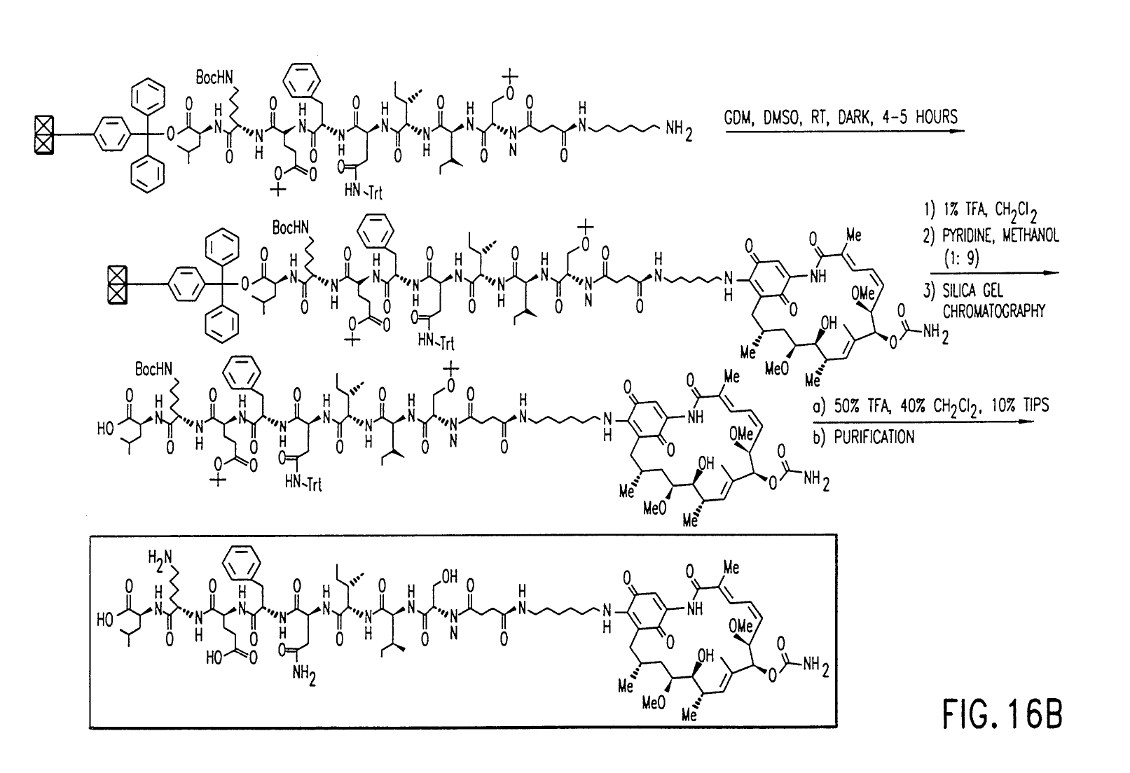

In nonlimiting embodiments of the invention, a fluorenylmethoxy carbonate (“Fmoc”) strategy may be used, wherein all deprotections and couplings are performed under basic conditions, compatible with the resin. FIG. 13A-B depict the synthesis of a protected peptide on TGT resin using Fmoc protecting groups (“PyBop” refers to benzotriazolyloxy-tris-pyrrolidino-phosphonium hexafluorophosphate and “DIPEA” refers to diisopropylethylamine). The resulting peptide is protected at both amino and carboxyl termini, and therefore may be used as a common intermediate for conjugation to benzoquinone ansamycin via either terminus. FIG. 16A-B depict a scheme in which a fully protected peptide, as produced according to FIG. 13A-B, is deprotected at the amino terminus and then reacted with a primary amine linker and geldanamycin.

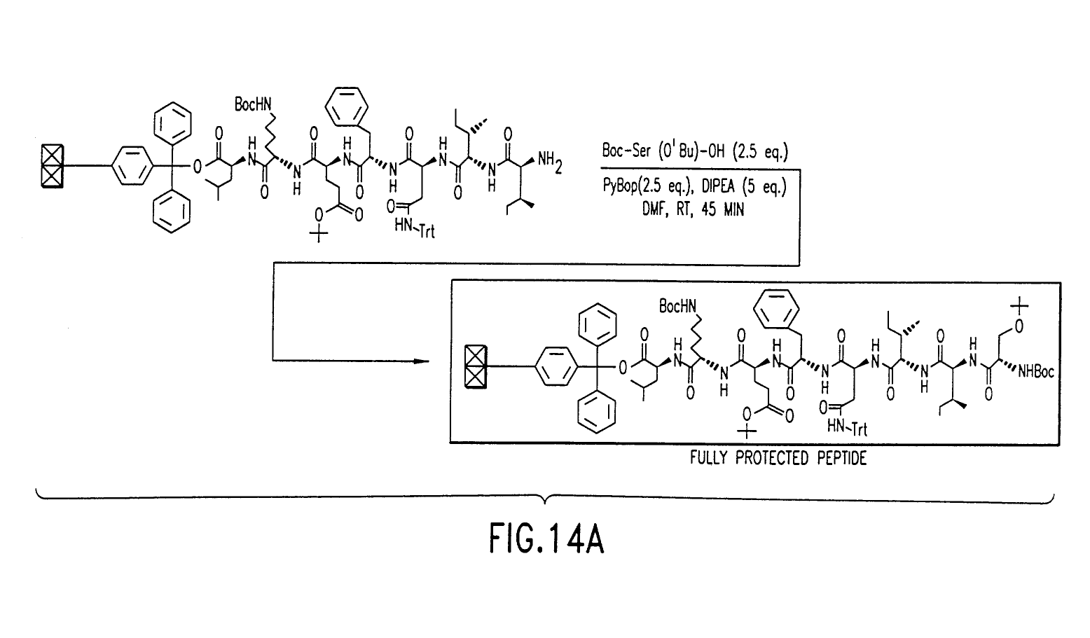

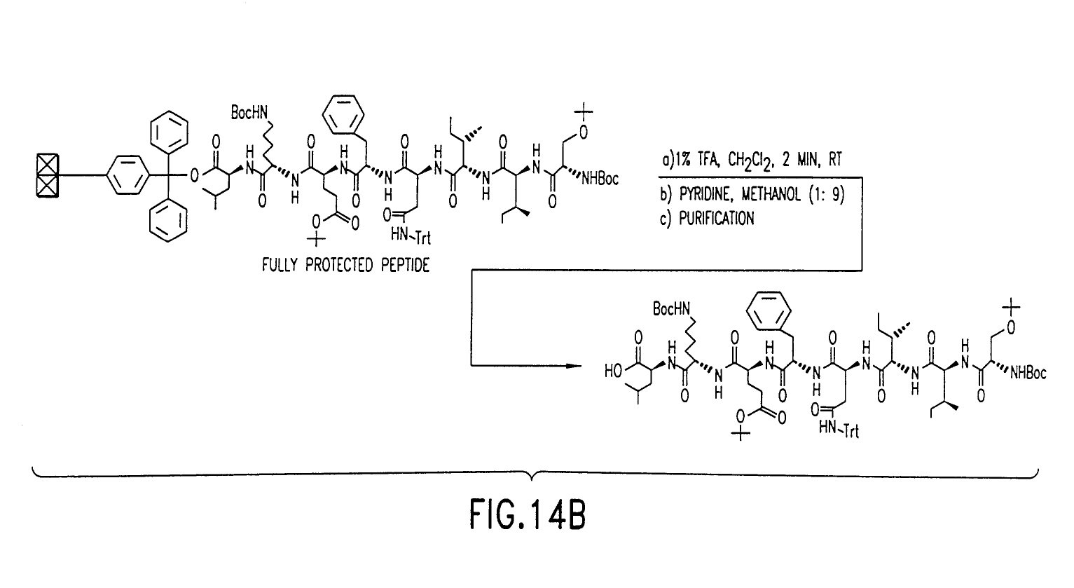

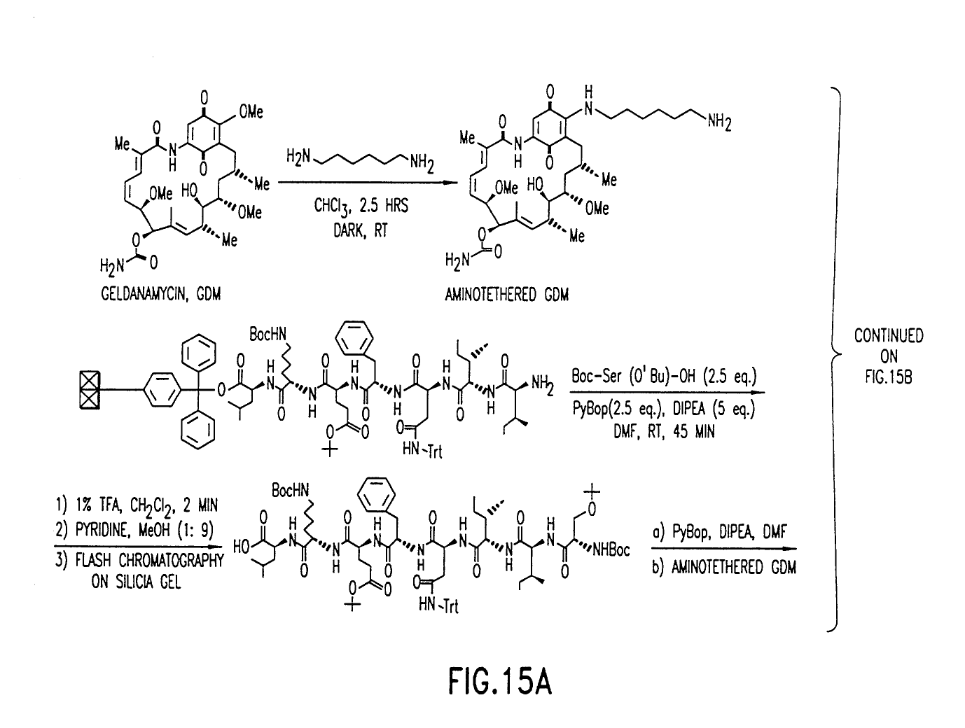

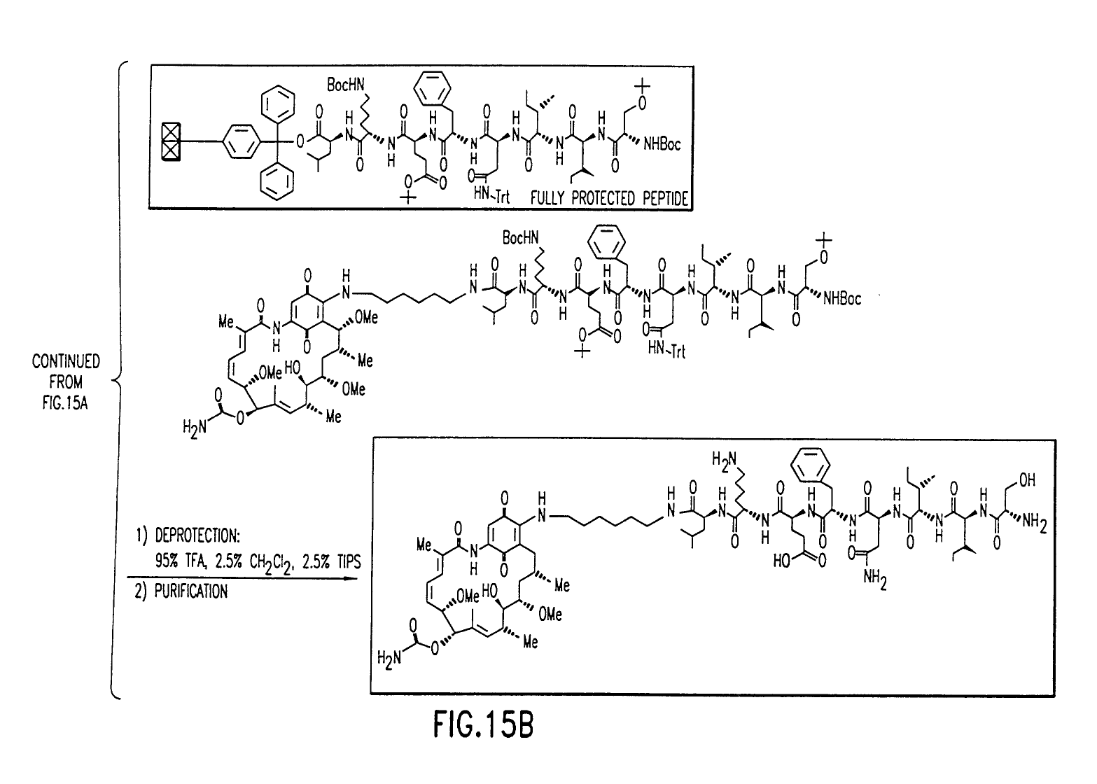

However, where antigenic peptide is to be conjugated to benzoquinone antibiotic via its carboxyl terminus it has been found to be preferable to add the last amino acid of the peptide as a N-Boc protected amino acid instead of a N-Fmoc protected amino acid (FIG. 14A). The resulting peptide has both carboxyl and amino termini protected (FIG. 14B), and thus may serve as a common intermediate for conjugation to antibiotic via either terminus. In FIG. 14B, the peptide is released from the resin, and its carboxyl terminus exposed, by treatment with 1% TFA, CH₂Cl₂, and then pyridine/methanol (1:9, volume:volume). A scheme whereby the resulting carboxyl-terminus deprotected (amino terminus protected) peptide is conjugated to geldanamycin is shown in FIG. 15. The N-Boc-based method has been found to greatly enhance the yields at the final deprotection step, probably because geldanamycin may be sensitive to excess piperidine required to remove the Fmoc. As shown in the last step of FIG. 15, once antigenic peptide has been conjugated to linker and geldanamycin via the peptide's carboxyl terminus, the remaining Boc protecting group on the amino terminus of the peptide may be removed without the use of piperidine.

It may also be useful to note that geldanamycin may be sensitive to extensive exposure to strong acids such as trifluoroacetic acid (“TFA”). For instance, stirring peptide having geldanamycin attached at its carboxyl terminus for four hours at room temperature in 50% TFA, 10% triisopropylsilane in CH₂Cl₂ yielded only trace amounts of the deprotected conjugate because of extensive product decomposition. In view of this problem, it may be desirable to use the following procedure as the final deprotection step (see FIG. 15). First, a conjugate peptide having a Boc-protected amino terminus may be treated with 95% trifluoroacetic acid (“TFA”), 2.5% triisopropylsilane, 2.5% CH₂Cl₂ for less than 1 hour. The above reagents should be initially added on ice and the reactions should be allowed to gradually warm to room temperature. After addition of water, the crude mixture may then be evaporated to dryness under high vacuum. The resulting purple solid may then be washed with chloroform and dissolved in water to produce a purple solution which may be pH adjusted to about 5 with triethylammonium bicarbonate, filtered, and submitted to HPLC.

The resulting conjugate peptide may be purified using any method known in the art (see Nishino et al., 1992, Tetrahedron Letts. 33:7007; Kuroda et al., 1992, Int. J. Peptide Prot. Res. 40:294; Alpert, 1990, J. Chromatography 499:177). Care should be taken not to use conditions which would substantially impair the biological function of either the hsp-binding portion or antigenic portion of the molecule. A specific, nonlimiting example of a method for the purification of conjugate peptide is as folows. The foregoing filtered solution, at pH 5, may be injected into a preconditioned HPLC column, such as a PolyHYDROXYETHYL Aspartamide™, from PolyLC. Columbia, Md. The conjugate peptide may then be eluted using a two-component elution system: eluent A=6.8% 10 mM triethylammonium acetate in 92% acetonitrile and 1.2% hexafluoroisopropanol; eluent B=10% 10 mM triethylammonium acetate, 10% acetonitrile in water. Reaction product may be injected into the column in 100% eluent A, eluent A may be kept isocratic at 3.2 ml/min for ten minutes, and then the proportion of eluent B may be increased over 40 minutes to 35%. At this stage the product eluted with a retention time of about 60 minutes.

Antigenic peptides according to the invention may be capable of inducing an immune response to any antigen of interest. Antigens of interest include, but are not limited to, antigens associated with neoplasia such as sarcoma, lymphoma, leukemia, melanoma, carcinoma of the breast, carcinoma of the prostate, ovarian carcinoma, carcinoma of the cervix, uterine carcinoma, colon carcinoma, carcinoma of the lung, glioblastoma, and astrocytoma, antigens associated with defective tumor suppressor genes such as p53; antigens associated with oncogenes such as ras, src, erbB, fos, abl, and myc; antigens associated with infectious diseases caused by a bacterium, virus, protozoan, mycoplasma, fingus, yeast, parasite or prion; and antigens associated with an allergy or autoimmune disease. Examples of sources of antigens associated with infectious disease include, but are not limited to, a human papilloma virus (see below), a herpes virus such as herpes simplex or herpes zoster, a retrovirus such as human immunodeficiency virus 1 or 2, a hepatitis virus, an influenza virus, a rhinovirus, a respiratory syncytial virus, a cytomegalovirus, an adenovirus, Mycoplasma pneumoniae, a bacterium of the genus Salmonella, Staphylococcus, Streptococcus, Enterococcus, Clostridium, Escherichia, Klebsiella, Vibrio, or Mycobacterium, and a protozoan such as an amoeba, a malarial parasite, and Trypanosoma cruzi.

Specific, nonlimiting examples of human papilloma virus antigenic peptides which may be comprised in a conjugate peptide of the invention are as follows:

| | |
|---|---|
| Leu Leu Leu Gly Thr Leu Asn Ile Val; | (SEQ ID NO: ) |
| Leu Leu Met Gly Thr Leu Gly Ile Val; | (SEQ ID NO: ) |
| Thr Leu Gln Asp Ile Val Leu His Leu; | (SEQ ID NO: ) |
| Gly Leu His Cys Tyr Glu Gln Leu Val; | (SEQ ID NO: ) |
| and | |
| Pro Leu Lys Gln His Phe Gln Ile Val. | (SEQ ID NO: ) |

Conjugate peptides of the invention may be prepared chemically or using recombinant techniques. To join tether and antigenic peptide, each peptide may be prepared separately and later covalently joined or, preferably, the two may be synthesized sequentially (although another peptide sequence may reside between tether and antigenic peptides) as comprised in a single molecule. In preferred, nonlimiting embodiments, the conjugate peptides may contain 15-40 amino acids, and more preferably 15-25 amino acids, and may further comprise lipid or carbohydrate moieties.

### 5.3. METHODS OF USING CONJUGATE PEPTIDES

The present invention provides for therapeutic compositions comprising conjugate peptides which may or may not also comprise heat shock protein, for compositions which result in the production of conjugate peptides in a subject, and for methods of using such compositions.

In particular embodiments, compositions of the invention comprise a therapeutically effective amount of a conjugate peptide in a suitable pharmaceutical carrier. Such compositions may further comprise other biologically active substances, including but not limited to cytokines and adjuvant compounds.

In further embodiments, compositions of the invention comprise a nucleic acid encoding a conjugate peptide comprised in a suitable expression vector, such that when the composition is administered to a subject the conjugate peptide is expressed.

In related embodiments, compositions of the invention comprise a cell containing a nucleic acid encoding a conjugate peptide, such that when the cell is introduced into a subject the conjugate peptide is expressed and released in the subject. Suitable cells include eukaryotic as well as prokaryotic cells.

According to additional embodiments, compositions of the invention comprise a conjugate peptide and a heat shock protein. Such compositions may further comprise one or more additional heat shock protein or protein which serves as an accessory in the chaperone process, and/or may comprise a lymphokine. In preferred nonlimiting embodiments of the invention, in such compositions the conjugate peptide is bound to the heat shock protein. Such binding may be achieved, in general under conditions where (i) the salt concentrations may be between 20-350 mM, preferably between 50-250 mM, and more preferably between 100-200 mM (of, for example,NaCl or KCl); (ii) temperature may be between 4-50° C., preferably between 10-40° C., and more preferably between 20-37° C.; and (iii) pH may be between 4-10, and preferably between 6-8 (all ranges inclusive of endpoints). In a specific, nonlimiting example of the invention, conjugate peptide may be bound to heat shock protein by mixing a molar ratio of 1:1 to 100:1 of conjugate peptide:heat shock protein, on ice, in a buffer which is 20 mM HEPES pH 7.0, 150 mM KCl, 10 mM (NH₄)₂SO₄, 2 mM MgCl₂ and 2 mM MgADP, pH 7.0, and then incubating the mixture for 30 minutes at 37° C. A working example of such binding is set forth in Section 7, below.

In other nonlimiting specific examples, the present invention provides for compositions comprising a conjugate peptide, a heat shock protein, and a benzoquinone ansamycin antibiotic such as herbimycin A or geldanamycin. The molar ratio of antibiotic to heat shock protein in such composition may be 1-50-fold, preferably 1-30-fold, and more preferably 10-20-fold.

Accordingly, one or more of the foregoing compositions may be administered to a subject in order to treat or prevent a neoplastic disease, an infectious disease, or an immunologic disease or disorder. In particular, such compositions may be used to induce a therapeutic immune response in a subject suffering from a neoplastic disease, an infectious disease, or an immunologic disease or disorder. Where the compositions are used to induce or augment a humoral or cellular immune response in a subject, the increase in immunity (measured, for example, by antibody titer, cytotoxic activity, cytokine release, or by increase in B cell or T cell populations associated with the desired response) may be at least 2-fold, preferably at least 3-fold, and more preferably at least 4-fold.

The compositions of the invention may be administered by any suitable route, including but not limited to subcutaneously, intradermally, intramuscularly, intravenously, orally, intranasally, or topically.

Neoplastic diseases which may be treated according to the invention include, but are not limited to, sarcoma, lymphoma, leukemia, melanoma, carcinoma of the breast, carcinoma of the prostate, ovarian carcinoma, carcinoma of the cervix, uterine carcinoma, colon carcinoma, carcinoma of the lung, glioblastoma, and astrocytoma.

Infectious diseases which may be treated according to the invention include, but are not limited to, diseases caused by a bacterium, virus, protozoan, mycoplasma, fungus, yeast, parasite or prion, such as a human papilloma virus, a herpes virus such as herpes simplex or herpes zoster, a retrovirus such as human immunodeficiency virus 1 or 2, a hepatitis virus, an influenza virus, a rhinovirus, a respiratory syncytial virus, a cytomegalovirus, an adenovirus, Mycoplasma pneumoniae, a bacterium of the genus Salmonella, Staphylococcus, Streptococcus, Enterococcus, Clostridium, Escherichia, Klebsiella, Vibrio, or Mycobacterium, or a protozoan such as an amoeba, a malarial parasite, or Trypanosoma cruzi.

Diseases of the immune system which may be treated according to the invention include, but are not limited to, inherited or acquired immune deficiencies where the capacity of the subject to mount an immune response is impaired. Examples of acquired immune deficiencies include AIDS and ARC and the impairment of immunity associated with various cancers. Alternatively, the method of the invention may be used to treat autoimmune diseases, such as rheumatoid arthritis, systemic lupus erythematosis, diabetes mellitus, thyroiditis, and multiple sclerosis. In such embodiments, the conjugate peptide and its interaction with heat shock protein, and/or the immunization protocol, may be designed such that immunization results in a decreased immune response; for example, the immune response may be decreased if repeated or prolonged exposure of the subject to conjugate peptide occurs.

### 6. EXAMPLE: IDENTIFICATION OF TETHERS

### 6.1. MATERIALS AND METHODS

Preparation of a gp96 expression vector. The mouse cDNA encoding mature gp96 (i.e., wherein the endoplasmic reticulum signal peptide has been removed) was incorporated into the pET 11a expression vector (Novagen) as follows. Gp96 cDNA insert was prepared by polymerase chain reaction (PCR) of a pRc/CMV clone containing the cDNA using the following oligonucleotide primers:

| | |
|---|---|
| AGATATACATATGGATGATGAAGTCGACGTGG and | (SEQ ID NO: ) |
| TCGGATCCTTACAATTCATCCTTCTCTGTAGATTC. | (SEQ ID NO: ) |

The resulting gp96 insert was then cut with NdeI and BamHI and repurified, and ligated into pET 11a which also had been cut with NdeI and BamHI and repurified, to form the expression vector pET11gp96.

Expression of gp96. pET11gp96 was transformed into BL21 Escherichia coli cells, and plated on LB plates containing ampicillin (50 μg/ml). One of the resulting colonies was used to inoculate a 20 ml overnight culture of 2×TY medium containing ampicillin (150 μg/ml). The following day, the resulting culture was spun down and the harvested bacteria were resuspended in 1 ml of fresh medium. Two one liter cultures were then each inoculated with 0.5 ml of the harvested cells and allowed to grow at 37° C. until the optical density, measured at 600 nm, was 0.5. Then, IPTG was added to a concentration of 1 mM and the cells were cultured for another 3 hours before being harvested by centrifligation. The resulting cell pellet was resuspended in 20 ml of 50 mM HEPES pH 7.5, 50 mM KCl, 5 mM MgAcetate, 20% sucrose and 1 mM PMSF. Cell extracts were prepared by pressure shearing in a French Press. The lysates were then spun at 100,000×g for 1.5 hours and the supernatant, which constituted crude gp96 extract, was collected.

Purification of gp96. The following steps were all performed at 4° C. A 12.5 cm×3.2 cm column of DE52 resin (Whatman) was equilibrated in a solution of 50 mM MOPS pH 7.4, 10 mM NaCl, 5 mM MgAcetate (hereafter, “Buffer A”). The crude gp96 extract was diluted 2-fold and immediately loaded onto the column at a flow rate of 2 ml/min. Elution from the column was achieved using a gradient of a solution of 50 mM MOPS pH 7.4, 1M NaCl, 5 mM MgAcetate (hereafter, “Buffer B”) from 0% to 100% Buffer B over 1000 ml. The elution profile was examined by subjecting fractions collected from the column to SDS-PAGE analysis. Fractions containing gp96 were pooled and diluted 2-fold with cold water, and were immediately run onto the next column (see below).

A 10 cm×1 cm column of hydroxyapatite (BioRad) was washed with 100 ml 0.5 M K₂HPO₄, 50 mM KCl pH 7.4 and then equilibrated with 10 mM K₂HPO₄, 50 mM KCl pH 7.4. The pooled diluted fractions from the DE52 column were loaded onto this column at a flow rate of 1 ml/min. The gp96 protein was eluted in a gradient of 10-500 mM K₂HPO₄ over 800 ml. Fractions containing gp96 were pooled and loaded onto the phenylsepharose column described below.

A 9 cm×3 cm column of phenylsepharose (Pharmacia) was equilibrated with 500 mM NaCl, 50 mM MOPS pH 7.4. The pooled fractions containing gp96 from the hydroxyapatite were loaded onto this column at 1 ml/min and the gp96 was eluted in a gradient of 500-0 mM NaCl over 800 ml. The gp96 containing fractions collected from the column were identified by SDS-PAGE, pooled, and concentrated.

The gp96 was then loaded onto a Hi Load 26/60 Superdex-200 column (Pharmacia) equilibrated with 100 mM NaCl, 5 mM MgAcetate, 50 mM MOPS pH 7.5. 3 ml fractions were collected, and the fractions containing the most pure gp96 (as identified by SDS PAGE using a 12 percent reducing gel) and pooled. To the pooled fractions, glycerol was added to 10% (v/v), and then the fractions were concentrated to 21 mg/ml on a Centricon-50 concentrator (Amicon), frozen using liquid nitrogen, and stored at −80° C.

Affinity panning. The Ph.D. Phage Display Library Kit (New England BioLabs, Beverly, Mass.), was used for affinity panning. For each panning experiment, a well of a 96-well polystyrene microtiter plate (each well having a 6 mm diameter) was filled with 150 μl of a solution of 200 μg/ml of gp96 in 0.1 M NaHCO₃ pH 8.3. If herbimycin A was to be included in the experiment, 1 μl of 10 mg/ml herbimycin A (GIBCO) in DMSO was added to each well, corresponding to a 50-fold molar excess relative to gp96. The plate was then held at 4° C. overnight in the dark (herbimycin is light sensitive). The next day, the gp96 solution was removed from the well and 200 μl of blocking buffer (0.1 M NaHCO₃ (pH 8.6), 5 mg/ml bovine serum albumin (BSA), 0.02% NaN₃) was added, and the plate containing the well was incubated at 4° C. for a further hour. The well was then washed six times with TBS (50 mM Tris-HCl (pH 7.5), 150 mM NaCl) further containing either 0.1%, 0.3% or 0.5% TWEEN 20 depending on whether the first, second, or third round, respectively, of panning was being performed. 2×10¹¹ phage were then diluted into 100 μl of the appropriate binding buffer (see below), containing the appropriate amount of TWEEN 20 for that particular round of panning and the phage were incubated in the well at 37° C. for 1 hour. Non-bound phage were then removed from the well, and the well was washed ten times with the particular binding buffer used for phage binding containing the appropriate amount of TWEEN 20 for that round of panning. Bound phage were then eluted by a 10 min. incubation in 100 μl of 0.2 M glycine pH 2.2. The eluate was then neutralized by adding 15 μl 1.5 M Tris pH 8.8. These phage were then amplified in two cycles of amplification, titered and used in the next round of panning. Three rounds of panning were performed. After the last round of panning, between ten and fifty phage clones from each experiment were sequenced and the corresponding peptide sequences were deduced.

### 6.2. RESULTS

Affinity panning was performed using a diversity of binding buffers, which differed in electrolyte concentration, calcium ion concentration, and/or the presence or absence of herbimycin A, dithiothreitol (“DTT”), or nucleotide. As discussed below, when the composition of binding buffer was varied, the composition of bound phage-expressed peptides was found to change.

Using the binding buffer utilized in Blond-Elguindi et al., 1993, Cell 75:717-728 (20 mM HEPES pH 7.5, 20 mM KCl, 10 mM (NH₄)₂SO₄, 2 mM MgCl₂, and 0.1%, 0.3% or 0.5% TWEEN-20, depending on the panning round), phage expressing the peptides set forth in Table I were found to bind to gp96. The percentage of specific amino acids occurring in these peptides is compared to the expected percentages (based on the occurrence of each amino acid in the expression library as a whole, provided by the manufacturer) in Table II. From these results, and not considering the relative positions of each amino acid in the bound peptides, it appears that binding to peptides containing aspartic acid, threonine, proline, tyrosine and phenylalanine (and, to a lesser extent, serine) was favored. Conversely, peptides containing glycine, glutamine, asparagine, leucine, isoleucine, and, to a lesser extent, alanine and valine were selected against.

TABLE I

| | |
|---|---|
| Tyr Thr Leu Val Gln Pro Leu | (SEQ ID NO: ) |
| Thr Pro Asp Ile Thr Pro Lys | (SEQ ID NO: ) |
| Thr Tyr Pro Asp Leu Arg Tyr | (SEQ ID NO: ) |
| Asp Arg Thr His Ala Thr Ser | (SEQ ID NO: ) |
| Met Ser Thr Thr Phe Tyr Ser | (SEQ ID NO: ) |
| Tyr Gln His Ala Val Gln Thr | (SEQ ID NO: ) |
| Phe Pro Phe Ser Ala Ser Thr | (SEQ ID NO: ) |
| Ser Ser Phe Pro Pro Leu Asp | (SEQ ID NO: ) |
| Met Ala Pro Ser Pro Pro His | (SEQ ID NO: ) |
| Ser Ser Phe Pro Asp Leu Leu | (SEQ ID NO: ) |

TABLE II

| A.A. | % actual | % expected |
|---|---|---|
| His | 4.28 | 4.3 |
| Arg | 2.85 | 3.9 |
| Lys | 1.4 | 1.7 |
| Gln | 4.28 | 6.4 |
| Asn | 0 | 4.1 |
| Asp | 7.14 | 2.1 |
| Glu | 0 | 1.2 |
| Leu | 8.57 | 11.8 |
| Ala | 5.7 | 7.2 |
| Val | 2.85 | 4.3 |
| Ile | 1.43 | 5.4 |
| Gly | 0 | 3.7 |
| Ser | 14.28 | 11.4 |
| Thr | 14.28 | 9.3 |
| Pro | 15.7 | 12 |
| Tyr | 7.14 | 2.9 |
| Phe | 7.14 | 2.9 |
| Trp | 0 | 1 |
| Cys | 0 | 0.8 |
| Met | 2.85 | 3.3 |

Tables IA and IIA, respectively, show that phage expressing peptides of a different composition bound to gp96 when the same binding buffer was used, but herbimycin A was present (where herbimycin A was added to gp96 during binding to the polystyrene well). The composition of bound peptides appeared to be enriched in histidine, alanine, and isoleucine (and to a lesser extent serine, arginine and tyrosine) residues.

TABLE IA

| | |
|---|---|
| His Ser Tyr Asn Arg Leu Pro | (SEQ ID NO: ) |
| His Leu Thr His Ser Gln Arg | (SEQ ID NO: ) |
| Gln Ala Ala Gln Ser Arg Ser | (SEQ ID NO: ) |
| Phe Ala Thr His His Ile Gly | (SEQ ID NO: ) |
| Ser Met Pro Glu Pro Leu Ile | (SEQ ID NO: ) |
| Ile Pro Arg Tyr His Leu Ile | (SEQ ID NO: ) |
| Ser Ala Pro His Met Thr Ser | (SEQ ID NO: ) |
| Lys Ala Pro Val Trp Ala Ser | (SEQ ID NO: ) |
| Leu Pro His Trp Leu Leu Ile | (SEQ ID NO: ) |
| Ala Ser Ala Gly Tyr Gln Ile | (SEQ ID NO: ) |

TABLE IIA

| A.A. | % actual | % expected |
|---|---|---|
| His | 11.4 | 4.3 |
| Arg | 5.7 | 3.9 |
| Lys | 1.4 | 1.7 |
| Gln | 5.7 | 6.4 |
| Asn | 1.4 | 4.1 |
| Asp | 0 | 2.1 |
| Glu | 1.4 | 1.2 |
| Leu | 10.0 | 11.8 |
| Ala | 11.4 | 7.2 |
| Val | 1.4 | 4.3 |
| Ile | 8.57 | 5.4 |
| Gly | 2.85 | 3.7 |
| Ser | 12.85 | 11.4 |
| Thr | 4.3 | 9.3 |
| Pro | 10.0 | 12 |
| Tyr | 4.28 | 2.9 |
| Phe | 1.4 | 2.9 |
| Trp | 2.85 | 1 |
| Cys | 0 | 0.8 |
| Met | 2.85 | 3.3 |

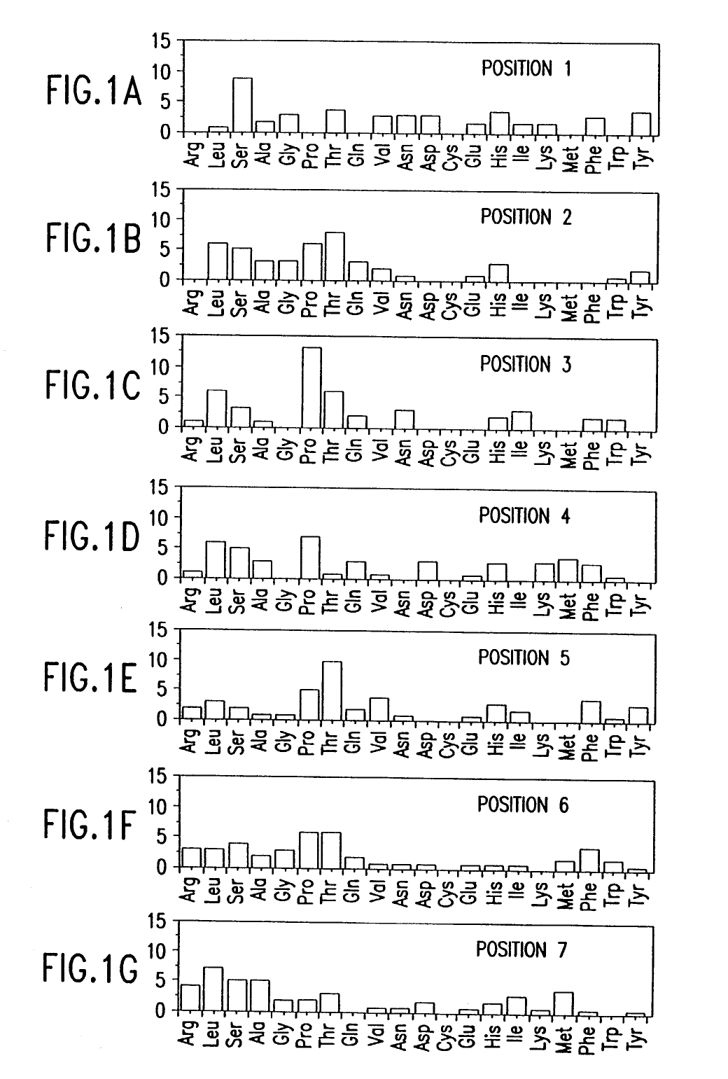

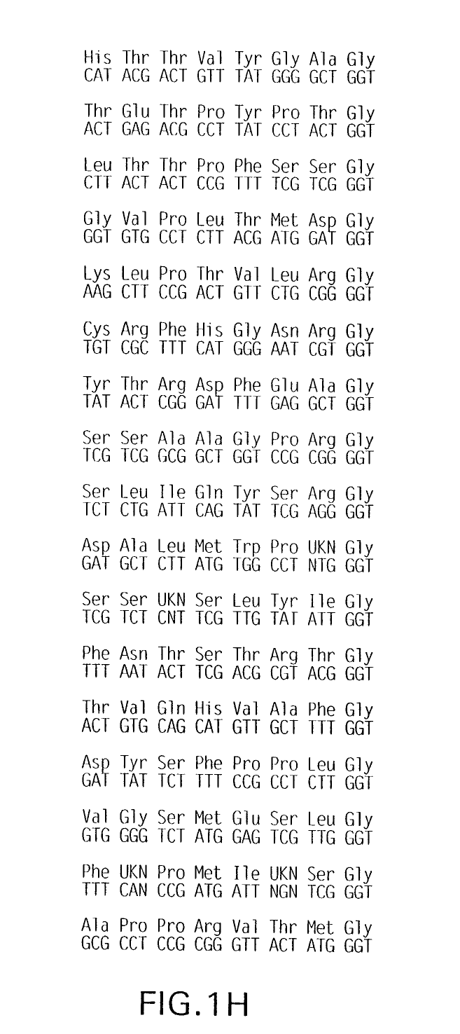

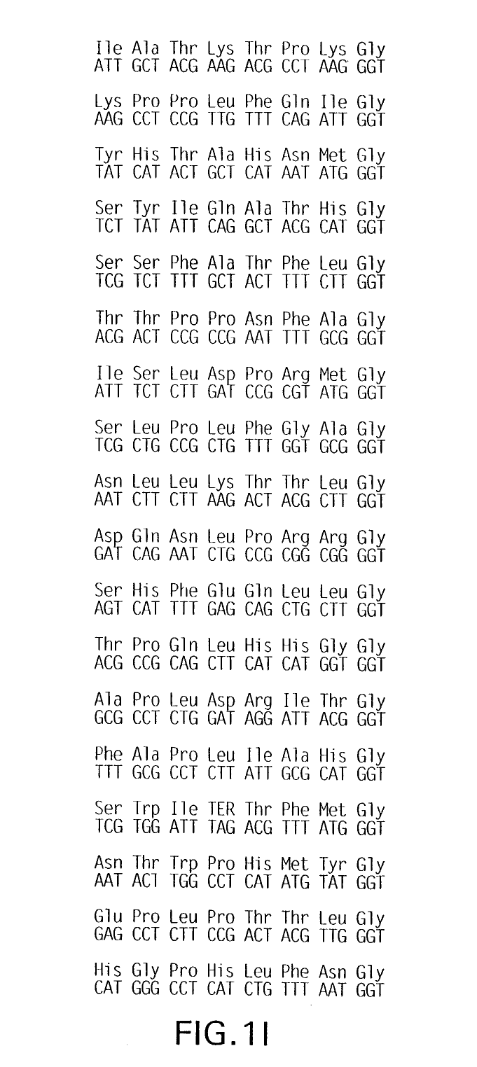

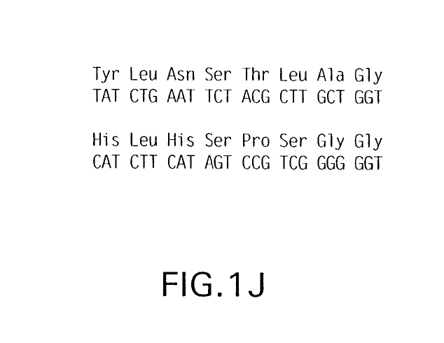

When the binding buffer was modified to contain the eletrolyte KCl in physiologic concentration (20 mM HEPES pH 7.5, 100 mM KCl, 1 mM MgAcetate and 0.1%, 0.3% or 0.5% TWEEN-20, depending on the panning round), and herbimycin A was present, the composition of phage-expressed peptindes bound was found to be enriched in threonine, phenylalanine and histidine, and relatively depleted for glutamine, isoleucine, and alanine residues. Tables III contains the sequences of 46 bound peptides; the degree of enrichment for certain amino acids in these 46 peptides is set forth in Table IV. FIG. 1H depicts the nucleic acid sequences encoding 37 of these peptides. FIG. 1A-G depicts the distribution of amino acids at positions 1-7, respectively, of the expressed peptides in all those phage sequenced, and shows that the occurrence of serine appeared to be favored at postion 1, proline was favored at position 3, and threonine was favored at position 5.

TABLE III

| | |
|---|---|
| Val Thr Pro Lys Thr Gly Ser | (SEQ ID NO: ) |
| Glu His Pro Met Pro Val Leu | (SEQ ID NO: ) |
| Val Ser Ser Phe Val Thr Ser | (SEQ ID NO: ) |
| Ser Thr His Phe Thr Trp Pro | (SEQ ID NO: ) |
| Gly Gln Trp Trp Ser Pro Asp | (SEQ ID NO: ) |
| Gly Pro Pro His Gln Asp Ser | (SEQ ID NO: ) |
| Asn Thr Leu Pro Ser Thr Ile | (SEQ ID NO: ) |
| His Gln Pro Ser Arg Trp Val | (SEQ ID NO: ) |
| Tyr Gly Asn Pro Leu Gln Pro | (SEQ ID NO: ) |
| His Thr Thr Val Tyr Gly Ala | (SEQ ID NO: ) |
| Thr Glu Thr Pro Tyr Pro Thr | (SEQ ID NO: ) |
| Leu Thr Thr Pro Phe Ser Ser | (SEQ ID NO: ) |
| Gly Val Pro Leu Thr Met Asp | (SEQ ID NO: ) |
| Lys Leu Pro Thr Val Leu Arg | (SEQ ID NO: ) |
| Cys Arg Phe His Gly Asn Arg | (SEQ ID NO: ) |
| Tyr Thr Arg Asp Phe Glu Ala | (SEQ ID NO: ) |
| Ser Ser Ala Ala Gly Pro Arg | (SEQ ID NO: ) |
| Ser Leu Ile Gln Tyr Ser Arg | (SEQ ID NO: ) |
| Asp Ala Leu Met Trp Pro UKN | (SEQ ID NO: ) |
| Ser Ser UKN Ser Leu Tyr Ile | (SEQ ID NO: ) |
| Phe Asn Thr Ser Thr Arg Thr | (SEQ ID NO: ) |
| Thr Val Gln His Val Ala Phe | (SEQ ID NO: ) |
| Asp Tyr Ser Phe Pro Pro Leu | (SEQ ID NO: ) |
| Val Gly Ser Met Glu Ser Leu | (SEQ ID NO: ) |
| Phe UKN Pro Met Ile UKN Ser | (SEQ ID NO: ) |
| Ala Pro Pro Arg Val Thr Met | (SEQ ID NO: ) |
| Ile Ala Thr Lys Thr Pro Lys | (SEQ ID NO: ) |
| Lys Pro Pro Leu Phe Gln Ile | (SEQ ID NO: ) |
| Tyr His Thr Ala His Asn Met | (SEQ ID NO: ) |
| Ser Tyr Ile Gln Ala Thr His | (SEQ ID NO: ) |
| Ser Ser Phe Ala Thr Phe Leu | (SEQ ID NO: ) |
| Thr Thr Pro Pro Asn Phe Ala | (SEQ ID NO: ) |
| Ile Ser Leu Asp Pro Arg Met | (SEQ ID NO: ) |
| Ser Leu Pro Leu Phe Gly Ala | (SEQ ID NO: ) |
| Asn Leu Leu Lys Thr Thr Leu | (SEQ ID NO: ) |
| Asp Gln Asn Leu Pro Arg Arg | (SEQ ID NO: ) |
| Ser His Phe Glu Gln Leu Leu | (SEQ ID NO: ) |
| Thr Pro Gln Leu His His Gly | (SEQ ID NO: ) |
| Ala Pro Leu Asp Arg Ile Thr | (SEQ ID NO: ) |
| Phe Ala Pro Leu Ile Ala His | (SEQ ID NO: ) |
| Ser Trp Ile TER Thr Phe Met | (SEQ ID NO: ) |
| Asn Thr Trp Pro His Met Tyr | (SEQ ID NO: ) |
| Glu Pro Leu Pro Thr Thr Leu | (SEQ ID NO: ) |
| His Gly Pro His Leu Phe Asn | (SEQ ID NO: ) |
| Tyr Leu Asn Ser Thr Leu Ala | (SEQ ID NO: ) |
| His Leu His Ser Pro Ser Gly | (SEQ ID NO: ) |

TABLE IV

| A.A. | % actual | % expected |
|---|---|---|
| His | 5.7 | 4.3 |
| Arg | 3.49 | 3.9 |
| Lys | 1.9 | 1.7 |
| Gln | 3.8 | 6.4 |
| Asn | 3.17 | 4.1 |
| Asp | 2.86 | 2.1 |
| Glu | 1.9 | 1.2 |
| Leu | 10.15 | 11.8 |
| Ala | 5.39 | 7.2 |
| Val | 3.8 | 4.3 |
| Ile | 3.49 | 5.4 |
| Gly | 3.8 | 3.7 |
| Ser | 10.47 | 11.4 |
| Thr | 12.06 | 9.3 |
| Pro | 12.38 | 12 |
| Tyr | 3.49 | 2.9 |
| Phe | 5.39 | 2.9 |
| Trp | 2.22 | 1 |
| Cys | 0 | 0.8 |
| Met | 3.17 | 3.3 |

The binding buffer used to generate the data of Tables III and IV was further modified to include 25 mM CaCl₂ (in order to simulate the high calcium concentration found in the endoplasmic reticulum), to produce a binding buffer having 20 mM HEPES pH 7.5, 100 mM KCl, 25 mM CaCl₂, and 5 mM MgAcetate and 0.1%, 0.3% or 0.5% TWEEN-20, depending on the panning round. The results of affinity panning using this binding buffer and gp96, in the presence of herbimycin A, are depicted in Tables V and VI. The data indicates that binding of phage expressing peptides containing phenylalanine, histidine, and tryptophan residues was favored. The sequence Phe-His-Trp-Trp-Trp (SEQ ID NO: ) appeared to be favored.

TABLE V

| | |
|---|---|
| Phe His Trp Trp Trp Gln Pro | (SEQ ID NO: ) |
| Ile Thr Leu Lys Tyr Pro Leu | (SEQ ID NO: ) |
| Phe His Trp Pro Trp Leu Phe | (SEQ ID NO: ) |
| Thr Ala Gln Asp Ser Thr Gly | (SEQ ID NO: ) |
| Phe His Trp Trp Trp Gln Pro | (SEQ ID NO: ) |
| Phe His Trp Trp Asp Trp Trp | (SEQ ID NO: ) |
| Glu Pro Phe Phe Arg Met Gln | (SEQ ID NO: ) |
| Thr Trp Trp Leu Asn Tyr Arg | (SEQ ID NO: ) |
| Phe His Trp Trp Trp Gln Pro | (SEQ ID NO: ) |
| Gln Pro Ser His Leu Arg Trp | (SEQ ID NO: ) |

TABLE VI

| A.A. | % actual | % expected |
|---|---|---|
| His | 8.6 | 4.3 |
| Arg | 4.3 | 3.9 |
| Lys | 1.4 | 1.7 |
| Gln | 8.6 | 6.4 |
| Asn | 1.4 | 4.1 |
| Asp | 2.85 | 2.1 |
| Glu | 1.4 | 1.2 |
| Leu | 7.1 | 11.8 |
| Ala | 1.4 | 7.2 |
| Val | 0 | 4.3 |
| Ile | 1.4 | 5.4 |
| Gly | 1.4 | 3.7 |
| Ser | 2.85 | 11.4 |
| Thr | 5.7 | 9.3 |
| Pro | 10.0 | 12 |
| Tyr | 2.85 | 2.9 |
| Phe | 11.4 | 2.9 |
| Trp | 25.7 | 1 |
| Cys | 0 | 0.8 |
| Met | 1.4 | 3.3 |

When the same binding buffer was used, but herbimycin A was not present, the composition of phage-expressed bound peptides was altered (Tables VA and VIA). In particular, the amount of serine and proline residues increased substantially, while the amount of tryptophan, though slightly decreased, remained high relative to its expected occurrence. The amount of phenylalanine decreased significantly but was still present at a frequency greater than expected.

TABLE VA

| | |
|---|---|
| Ser Pro Ala Ser Pro Val Tyr | (SEQ ID NO:) |
| Phe His Trp Trp Trp Gln Pro | (SEQ ID NO:) |
| His Pro Ser Asn Gln Ala Ser | (SEQ ID NO:) |
| Asn Ser Ala Pro Arg Pro Val | (SEQ ID NO:) |
| Gln Leu Trp Ser Ile Tyr Pro | (SEQ ID NO:) |
| Ser Trp Pro Phe Phe Asp Leu | (SEQ ID NO:) |
| Asp Thr Thr Leu Pro Leu His | (SEQ ID NO:) |
| Trp His Trp Gln Met Leu Trp | (SEQ ID NO:) |
| Asp Ser Phe Arg Thr Pro Val | (SEQ ID NO:) |
| Thr Ser Pro Leu Ser Leu Leu | (SEQ ID NO:) |

TABLE VIA

| A.A. | % actual | % expected |
|---|---|---|
| His | 5.7 | 4.3 |
| Arg | 2.8 | 3.9 |
| Lys | 0 | 1.7 |
| Gln | 5.7 | 6.4 |
| Asn | 2.8 | 4.1 |
| Asp | 4.3 | 2.1 |
| Glu | 0 | 1.2 |
| Leu | 11.4 | 11.8 |
| Ala | 4.3 | 7.2 |
| Val | 4.3 | 4.3 |
| Ile | 1.4 | 5.4 |
| Gly | 0 | 3.7 |
| Ser | 14.3 | 11.4 |
| Thr | 5.7 | 9.3 |
| Pro | 15.7 | 12 |
| Tyr | 2.8 | 2.9 |
| Phe | 5.7 | 2.9 |
| Trp | 11.4 | 1 |
| Cys | 0 | 0.8 |
| Met | 1.4 | 3.3 |

When an otherwise comparable binding buffer having a lower calcium ion concentration was used, the prevalence of tryptophan and phenylalanine residues decreased substantially, whereas the percentage of proline residues remained elevated. In particular, the use of a binding buffer having 20 mM HEPES pH 7.5, 100 mM KCl, 1 mM CaAcetate, 1 mM MgAcetate, and 0.1%, 0.3%, or 0.5% of TWEEN 20, depending on the panning round, and gp96 in the presence of herbimycin, yielded the results set forth in Tables VII and VIII.

TABLE VII

| | |
|---|---|
| Ala Tyr Asn Tyr Val Ser Asp | (SEQ ID NO:) |
| Arg Pro Leu His Asp Pro Met | (SEQ ID NO:) |
| Trp Pro Ser Thr Thr Leu Phe | (SEQ ID NO:) |
| Ala Thr Leu Glu Pro Val Arg | (SEQ ID NO:) |
| Ser Met Thr Val Leu Arg Pro | (SEQ ID NO:) |
| Gln Ile Gly Ala Pro Ser Trp | (SEQ ID NO:) |
| Ala Pro Asp Leu Tyr Val Pro | (SEQ ID NO:) |
| Arg Met Pro Pro Leu Leu Pro | (SEQ ID NO:) |
| Ala Lys Ala Thr Pro Glu His | (SEQ ID NO:) |

TABLE VIII

| A.A. | % actual | % expected |
|---|---|---|
| His | 3.17 | 4.3 |
| Arg | 6.35 | 3.9 |
| Lys | 1.58 | 1.7 |
| Gln | 1.58 | 6.4 |
| Asn | 1.58 | 4.1 |
| Asp | 4.76 | 2.1 |
| Glu | 3.17 | 1.2 |
| Leu | 11.1 | 11.8 |
| Ala | 9.5 | 7.2 |
| Val | 6.35 | 4.3 |
| Ile | 1.58 | 5.4 |
| Gly | 1.58 | 3.7 |
| Ser | 6.35 | 11.4 |
| Thr | 7.94 | 9.3 |
| Pro | 19.0 | 12 |
| Tyr | 4.76 | 2.9 |
| Phe | 1.58 | 2.9 |
| Trp | 3.17 | 1 |
| Cys | 0 | 0.8 |
| Met | 4.76 | 3.3 |

Affinity panning experiments were also carried out using a binding buffer having, in addition to physiologic electrolyte levels and a low calcium concentration, DTT (in order to create a reducing environment). The results of such experiments, using, as binding buffer, 20 mM HEPES pH 7.5, 100 mM KCl, 1 mM CaCl₂, 1 mM DTT, and 1 mM MgAcetate, with 0.1%, 0.3% or 0.5% TWEEN 20, depending on the panning round, and gp96 with herbimycin A, as hsp target, are shown in Tables IX and X. Phage-expressed peptides binding to gp96 under these conditions were enriched for histidine, arginine, leucine and proline residues, and were somewhat enriched for asparagine and tyrosine residues.

TABLE IX

| | |
|---|---|
| Thr Pro Pro Leu Arg Ile Asn | (SEQ ID NO:) |
| Leu Pro Ile His Ala Pro His | (SEQ ID NO:) |
| Asp Leu Asn Ala Tyr Thr His | (SEQ ID NO:) |
| Val Thr Leu Pro Asn Phe His | (SEQ ID NO:) |
| Asn Ser Arg Leu Pro Thr Leu | (SEQ ID NO:) |
| Tyr Pro His Pro Ser Arg Ser | (SEQ ID NO:) |
| Gly Thr Ala His Phe Met Tyr | (SEQ ID NO:) |
| Tyr Ser Leu Leu Pro Thr Arg | (SEQ ID NO:) |
| Leu Pro Arg Arg Thr Leu Leu | (SEQ ID NO:) |

TABLE X

| A.A. | % actual | % expected |
|---|---|---|
| His | 9.5 | 4.3 |
| Arg | 9.5 | 3.9 |
| Lys | 0 | 1.7 |
| Gln | 0 | 6.4 |
| Asn | 6.3 | 4.1 |
| Asp | 1.58 | 2.1 |
| Glu | 0 | 1.2 |
| Leu | 17.4 | 11.8 |
| Ala | 4.76 | 7.2 |
| Val | 1.58 | 4.3 |
| Ile | 3.17 | 5.4 |
| Gly | 1.58 | 3.7 |
| Ser | 6.3 | 11.4 |
| Thr | 11.1 | 9.3 |
| Pro | 15.87 | 12 |
| Tyr | 6.3 | 2.9 |
| Phe | 3.17 | 2.9 |
| Trp | 0 | 1 |
| Cys | 0 | 0.8 |
| Met | 1.58 | 3.3 |

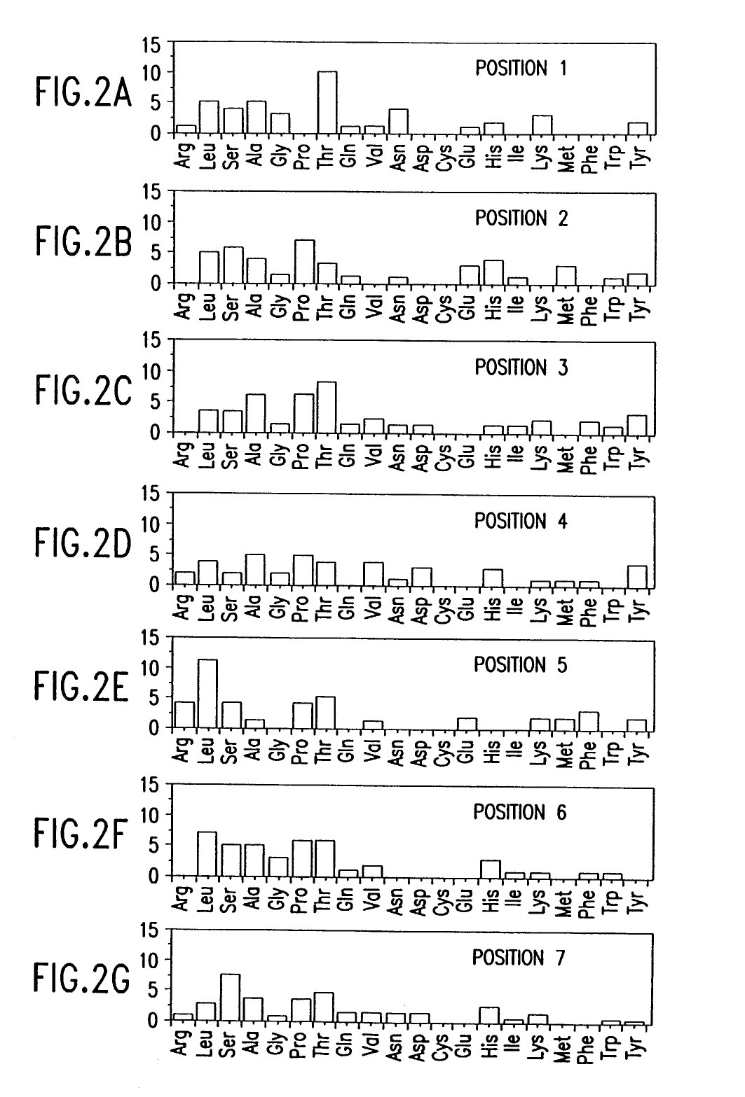

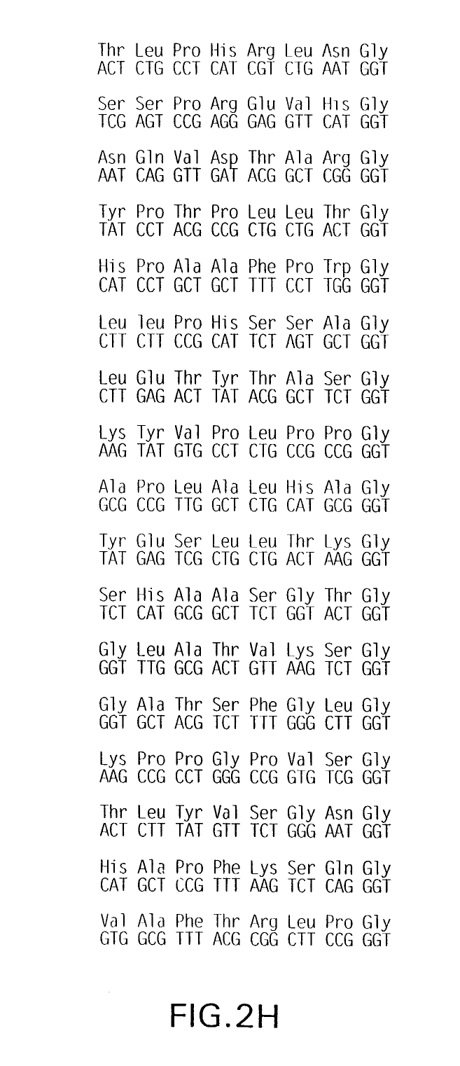

When calcium was eliminated from the binding buffer, such that affinity panning was carried out using, as hsp target, gp96 and herbimycin A, and, as binding buffer, 20 mM HEPES pH 7.5, 100 mM KCl, 1 mM DTT, 1 mM MgAcetate, and 0.1%, 0.3%, or 0.5% TWEEN 20 depending on the panning round, and 42 phage-expressed peptides were sequenced, results as set forth in Tables XI and XII were obtained. The binding of phage-expressed peptides containing threonine, serine, tyrosine, and, to a lesser extent, lysine, glutamic acid and leucine, appeared to be favored. When the distribution of amino acids at each of the seven positions of the expressed heptapeptide of all phage inserts sequenced were analyzed (see FIG. 2A-G, positions 1-7, respectively), the occurrence of threonine at positions 1 and 3, leucine at position 5 and serine at position 7 were favored. FIG. 2H shows nucleic acid sequences encoding 33 of these peptides.

TABLE XI

| | |
|---|---|
| Thr Ser Thr Leu Leu Trp Lys | (SEQ ID NO:) |
| Thr Ser Asp Met Lys Pro His | (SEQ ID NO:) |
| Thr Ser Ser Tyr Leu Ala Leu | (SEQ ID NO:) |
| Asn Leu Tyr Gly Pro His Asp | (SEQ ID NO:) |
| Leu Glu Thr Tyr Thr Ala Ser | (SEQ ID NO:) |
| Ala Tyr Lys Ser Leu Thr Gln | (SEQ ID NO:) |
| Ser Thr Ser Val Tyr Ser Ser | (SEQ ID NO:) |
| Glu Gly Pro Leu Arg Ser Pro | (SEQ ID NO:) |
| Thr Thr Tyr His Ala Leu Gly | (SEQ ID NO:) |
| Thr Leu Pro His Arg Leu Asn | (SEQ ID NO:) |
| Ser Ser Pro Arg Glu Val His | (SEQ ID NO:) |
| Asn Gln Val Asp Thr Ala Arg | (SEQ ID NO:) |
| Tyr Pro Thr Pro Leu Leu Thr | (SEQ ID NO:) |
| His Pro Ala Ala Phe Pro Trp | (SEQ ID NO:) |
| Leu Leu Pro His Ser Ser Ala | (SEQ ID NO:) |
| Leu Glu Thr Tyr Thr Ala Ser | (SEQ ID NO:) |
| Lys Tyr Val Pro Leu Pro Pro | (SEQ ID NO:) |
| Ala Pro Leu Ala Leu His Ala | (SEQ ID NO:) |
| Tyr Glu Ser Leu Leu Thr Lys | (SEQ ID NO:) |
| Ser His Ala Ala Ser Gly Thr | (SEQ ID NO:) |
| Gly Leu Ala Thr Val Lys Ser | (SEQ ID NO:) |
| Gly Ala Thr Ser Phe Gly Leu | (SEQ ID NO:) |
| Lys Pro Pro Gly Pro Val Ser | (SEQ ID NO:) |
| Thr Leu Tyr Val Ser Gly Asn | (SEQ ID NO:) |
| His Ala Pro Phe Lys Ser Gln | (SEQ ID NO:) |
| Val Ala Phe Thr Arg Leu Pro | (SEQ ID NO:) |
| Leu Pro Thr Arg Thr Pro Ala | (SEQ ID NO:) |
| Ala Ser Phe Asp Leu Leu Ile | (SEQ ID NO:) |
| Arg Met Asn Thr Glu Pro Pro | (SEQ ID NO:) |
| Lys Met Thr Pro Leu Thr Thr | (SEQ ID NO:) |
| Ala Asn Ala Thr Pro Leu Leu | (SEQ ID NO:) |
| Thr Ile Trp Pro Pro Pro Val | (SEQ ID NO:) |
| Gln Thr Lys Val Met Thr Thr | (SEQ ID NO:) |
| Asn His Ala Val Phe Ala Ser | (SEQ ID NO:) |
| Leu His Ala Ala UKN Thr Ser | (SEQ ID NO:) |
| Thr Trp Gln Pro Tyr Phe His | (SEQ ID NO:) |
| Ala Pro Leu Ala Leu His Ala | (SEQ ID NO:) |
| Thr Ala His Asp Leu Thr Val | (SEQ ID NO:) |
| Asn Met Thr Asn Met Leu Thr | (SEQ ID NO:) |
| Gly Ser Gly Leu Ser Gln Asp | (SEQ ID NO:) |
| Thr Pro Ile Lys Thr Ile Tyr | (SEQ ID NO:) |
| Ser His Leu Tyr Arg Ser Ser | (SEQ ID NO:) |

TABLE XII

| A.A. | % actual | % expected |
|---|---|---|
| His | 5.44 | 4.3 |
| Arg | 2.72 | 3.9 |
| Lys | 3.74 | 1.7 |
| Gln | 2.0 | 6.4 |
| Asn | 3.06 | 4.1 |
| Asp | 2.04 | 2.1 |
| Glu | 2.0 | 1.2 |
| Leu | 12.92 | 11.8 |
| Ala | 10.2 | 7.2 |
| Val | 4.08 | 4.3 |
| Ile | 1.36 | 5.4 |
| Gly | 3.74 | 3.7 |
| Ser | 10.88 | 11.4 |
| Thr | 13.95 | 9.3 |
| Pro | 10.88 | 12 |
| Tyr | 4.76 | 2.9 |
| Phe | 2.38 | 2.9 |
| Trp | 1.36 | 1 |
| Cys | 0 | 0.8 |
| Met | 2.0 | 3.3 |

Affinity panning was also performed using gp96, in the presence of herbimycin A, as hsp target, and, as binding buffer, the following solution, containing ATP: 20 mM HEPES pH 7.5, 100 mM KCl, 1 mM CaCl₂, 1 mM MgAcetate, 1 mM ATP, and 0.1%, 0.3% or 0.5% TWEEN 20, depending on the round of panning. The results are presented in Tables XIII and XIV. Phage-expressed peptides bound by gp96/herbimycin A under these conditions were enriched in histidine, tyrosine and serine (and to a lesser extent proline and tryptophan) residues.

TABLE XIII

| | |
|---|---|
| Val Ser Ile Gly His Pro Ser | (SEQ ID NO:) |
| Thr His Ser His Arg Pro Ser | (SEQ ID NO:) |
| Ile Thr Asn Pro Leu Thr Thr | (SEQ ID NO:) |
| Ser Ile Gln Ala His His Ser | (SEQ ID NO:) |
| Leu Asn Trp Pro Arg Val Leu | (SEQ ID NO:) |
| Tyr Tyr Tyr Ala Pro Pro Pro | (SEQ ID NO:) |
| Ser Leu Trp Thr Arg Leu Pro | (SEQ ID NO:) |
| Asn Val Tyr His Ser Ser Leu | (SEQ ID NO:) |

TABLE XIV

| A.A. | % actual | % expected |
|---|---|---|
| His | 10.7 | 4.3 |
| Arg | 5.35 | 3.9 |
| Lys | 0 | 1.7 |
| Gln | 1.78 | 6.4 |
| Asn | 5.3 | 4.1 |
| Asp | 0 | 2.1 |
| Glu | 0 | 1.2 |
| Leu | 10.7 | 11.8 |
| Ala | 3.57 | 7.2 |
| Val | 5.3 | 4.3 |
| Ile | 5.35 | 5.4 |
| Gly | 1.78 | 3.7 |
| Ser | 16.0 | 11.4 |
| Thr | 8.9 | 9.3 |
| Pro | 14.2 | 12 |
| Tyr | 7.1 | 2.9 |
| Phe | 0 | 2.9 |
| Trp | 3.57 | 1 |
| Cys | 0 | 0.8 |
| Met | 0 | 3.3 |

When, instead of ATP, the binding buffer contained AMP-PNP (20 mM HEPES pH 7.5, 100 mM KCl, 1 mM CaCl₂, 1 mnM MgAcetate, 1 mM AMP-PNP, and 0.1%, 0.3% or 0.5% TWEEN 20 depending on the panning round), as shown in Tables XV and XVI, binding of phage-expressed peptides containing histidine and valine. Position 4 appears to favor basic residues.

TABLE XV

| | |
|---|---|
| Asn Ser Pro His Pro Pro Thr | (SEQ ID NO:) |
| Val Pro Ala Lys Pro Arg His | (SEQ ID NO:) |
| His Asn Leu His Pro Asn Arg | (SEQ ID NO:) |
| Tyr Thr Thr His Arg Trp Leu | (SEQ ID NO:) |
| Ala Val Thr Ala Ala Ile Val | (SEQ ID NO:) |
| Thr Leu Met His Asp Arg Val | (SEQ ID NO:) |
| Thr Pro Leu Lys Val Pro Tyr | (SEQ ID NO:) |
| Phe Thr Asn Gln Gln Tyr His | (SEQ ID NO:) |
| Ser His Val Pro Ser Met Ala | (SEQ ID NO:) |
| His Gly Gln Ala Trp Gln Phe | (SEQ ID NO:) |

TABLE XVI

| A.A. | % actual | % expected |
|---|---|---|
| His | 12.8 | 4.3 |
| Arg | 5.7 | 3.9 |
| Lys | 2.85 | 1.7 |
| Gln | 5.7 | 6.4 |
| Asn | 5.7 | 4.1 |
| Asp | 1.4 | 2.1 |
| Glu | 0 | 1.2 |
| Leu | 5.7 | 11.8 |
| Ala | 8.5 | 7.2 |
| Val | 8.5 | 4.3 |
| Ile | 1.4 | 5.4 |
| Gly | 1.4 | 3.7 |
| Ser | 4.28 | 11.4 |
| Thr | 10 | 9.3 |
| Pro | 12.8 | 12 |
| Tyr | 4.28 | 2.9 |
| Phe | 2.85 | 2.9 |
| Trp | 2.85 | 1 |
| Cys | 0 | 0.8 |
| Met | 2.85 | 3.3 |

### 7. EXAMPLE: CONJUGATE PEPTIDE ADMINISTERED WITHOUT HEAT SHOCK PROTEIN INDUCES IMMUNITY

### 7.1. MATERIALS AND METHODS

Preparation of hsp70. Purified mouse cytosolic hsp70 was prepared from Escherichia coli DH5α cells transformed with pMS236 (Hunt and Calderwood, 1990, Gene 87:199-204) encoding mouse cytosolic hsp70. The cells were grown to an optical density of 0.6 at 600 nm at 37° C., and expression was induced by the addition of IPTG to a final concentration of 1 mM. Cells were harvested by centrifugation at 2-5 hours post-induction, and the cell pellets were resuspended to a volume of 20 ml with Buffer X (20 mM HEPES pH 7.0, 25 mM KCl, 1 mM DTT, 10 mM (NH₄)₂SO₄, 1 mM PMSF). The cells were lysed by passage (three times) through a French press. The lysate was cleared by low speed centrifugation, followed by centrifugation at 100,000×g for 30 minutes. The resulting cleared lysate was applied to a Pharmacia XK26 column packed with 100 ml DEAE Sephacel (Pharmacia) and equilibrated with Buffer X at a flow rate of 0.6 cm/min. The column was washed to stable baseline with Buffer X and eluted with Buffer X containing 175 mM KCl. The eluate was applied to a 25 ml ATP-agarose column (Sigma Chemical Co., A2767), washed to baseline with Buffer X, and eluted with Buffer X containing 1 mM MgATP preadjusted to pH 7.0. EDTA was added to the eluate to a final concentration of 2 mM. The eluate, which contained essentially pure hsp70, was precipitated by addition of (NH₄)₂SO₄ to 80 percent saturation. The precipitate was resuspended in Buffer X containing 1 mM MgCl₂ and dialyzed against the same buffer with multiple changes. For storage, the hsp70 was frozen in small aliquots at −70° C.

Peptides. The following peptides were prepared:

(i) OVA peptide (Ser Ile Ile Asn Phe Glu Lys Leu; SEQ ID NO: ); and (ii) OVA peptide joined, via a tripeptide linker (gly ser gly) to the BiP-binding tether peptide His Trp Asp Phe Ala Trp Pro Trp (Blond-Elguindi et al., 1993 Cell 75:717-728; SEQ ID NO: ), to form the conjugate peptide OVA-BiP (Ser Ile Ile Asn Phe Glu Lys Leu Gly Ser Gly His Trp Asp Phe Ala Trp Pro Trp; SEQ ID NO: ).

Preparation of hsp70 and/or peptide for use in immunization. Approximately 15 μg hsp70 and 12 μg OVA-BiP were mixed, on ice, to a final volume of 10 μL in Buffer Y (to produce a final concentration of 21.5 μM hsp70, 0.5 mM OVA-BiP, 20 mM HEPES pH 7.0, 150 mM KCl, 10 mM (NH₄)₂SO₄, 2 mM MgCl₂ and 2 mM MgADP, pH 7.0). The mixture was incubated for 30 minutes at 37° C. and was used for in vivo immunizations. Similar incubations were carried out with (i) 5 μl TiterMax adjuvant (Vaxcell, Norcross, Ga.) and 12 μg OVA-BiP (ii) 5 μl TiterMax and 5 μg OVA peptide or (iii) 12 μg OVA-BiP alone.

Preparation of cells for chromium release assay. Female C57BL/6 mice, 8-10 weeks old (two per assay), were immunized intradermally once (x1) or twice (x2) at a one-week interval with 10 μl of either (i) hsp70/OVA-BiP; (ii) TiterMax/OVA-BiP; (iii) TiterMax/OVA; or (iv) OVA-BiP. One week after the last immunization, the mice were sacrificed, their spleens removed, and used to prepare mononuclear effector cells. 8-10×10⁷ of these effector cells were then cultured with 4×107 gamma-irradiated (3000 rad) stimulator cells and feeder cells (which were obtained from the spleens of naive mice and sensitized, in vitro, with 10 μg/ml OVA peptide for 30 minutes at room temperature prior to gamma irradiation) in RPMI 1640 medium containing ten percent fetal calf serum, 100 U/ml penicillin (GIBCO, Cat. No. 15140-122), 100 μg/ml streptomycin, and 2 mM L-glutarnine. After culturing in vitro for five days, the cytotoxic activity of the resulting effector cells was assayed as set forth below. CTL lines were maintained by stimulation with irradiated stimulators, syngeneic splenic feeder cells plus T cell growth factors.

Chromium release assay. The cytotoxicity of spleen cells from immunized mice, cultured as set forth in the preceding paragraph, was assayed in a 4 hour ⁵¹Cr release assay using, as target cells, either (i) OVA-peptide pulsed EL4 cells or (ii) naive EL4 cells, which were chromium labeled. Effector cells were prepared as set forth above. Target cells were prepared as follows. EL4 cells were washed with PBS three times. To prepare naive cells, 5×10⁶ EL4 cells were incubated with 100 μCi ⁵¹Cr (sodium chromate, DuPont, Boston, Mass.) in 1 ml of 10% FCS/RPMI medium for 1 hour at 37° C. To prepare pulsed cells, 5×10⁶ EL4 cells were incubated with 1 μg/ml of OVA-peptide and 100 μCi ⁵¹Cr in 1 ml of 10% FCS/RPMI medium for 1 hour at 37° C. The target cells were then washed three times with RPMI, and resuspended to a final cell count of 1×10⁵ cells/ml in 10% FCS/RPMI. 10⁴ of the ⁵¹Cr-labeled EL-4 cells were mixed with effector lymphocytes to yield several effector to target cell (E/T) ratios, and then incubated for 4 hours. Supemnatants were harvested and radioactivitiy released by cytotoxic activity was measured in a gamma counter. The percent specific lysis was calculated as 100×[(cpm release by CTL—cpm spontaneously released)/(cpm maximal release—cpm spontaneously released)]. Maximal release was determined by adding 1% NP-40 to lyse all cells. Spontaneous release of all target in the absence of effector cells (measured in a culture of target cells (in the absence of effector cells) maintained in parallel for the duration of the assay) was less than 20% of the maximal release.

### 7.2. RESULTS AND DISCUSSION

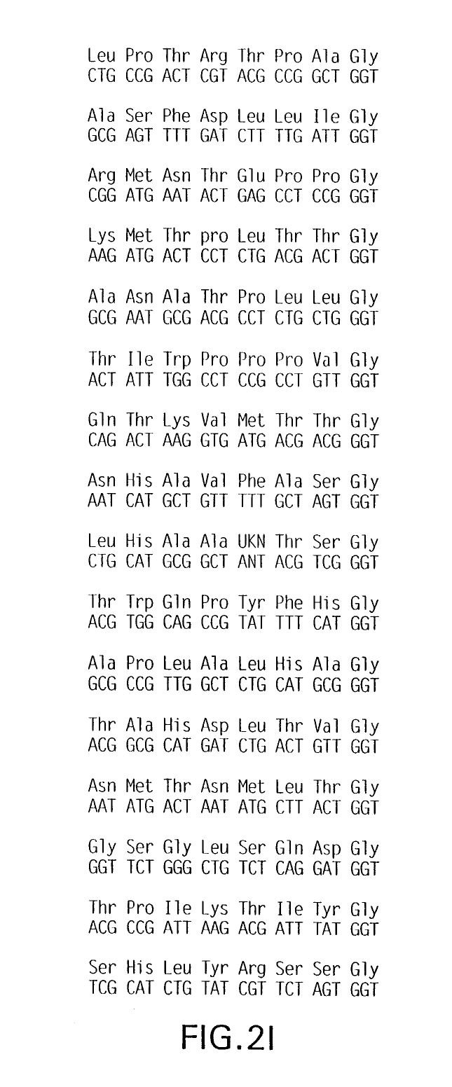

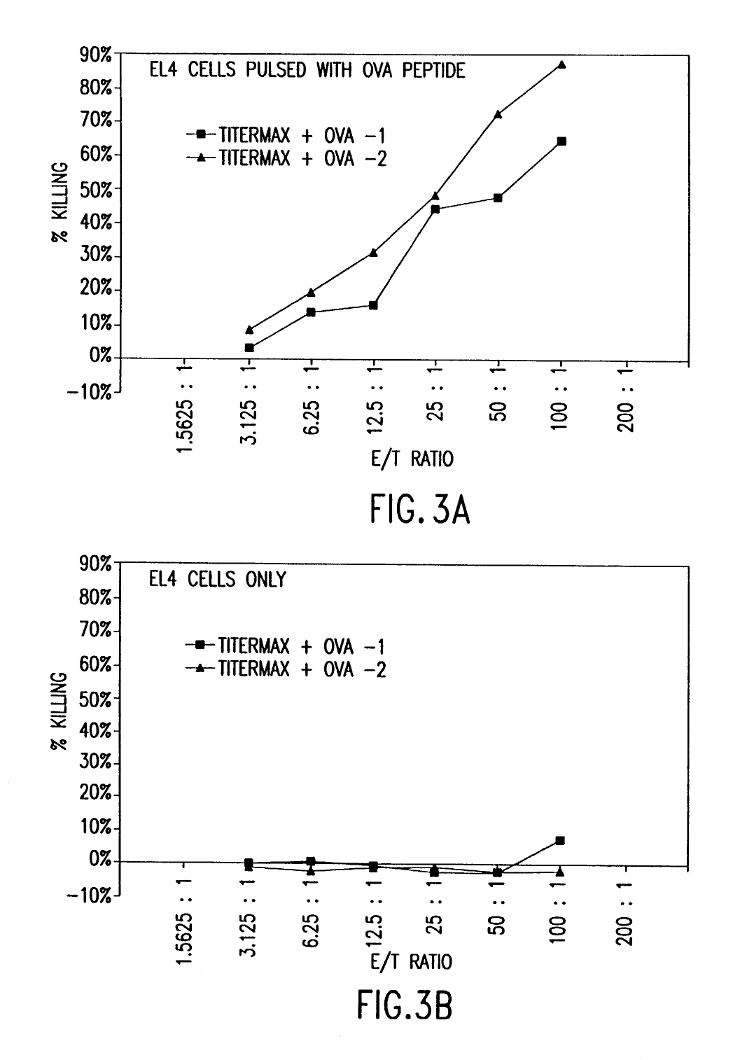

FIG. 3A-B depicts the cytotoxic activity of effector cells prepared from mice immunized once with TiterMax plus OVA peptide (which does not comprise a tether) against OVA-primed EL-4 target cells (FIG. 3A) or unprimed EL-4 control cells (FIG. 3B). The two curves represent data obtained with two different mice. These results indicate that TiterMax adjuvant together with OVA peptide was able to induce an OVA-specific cytotoxic immune response.

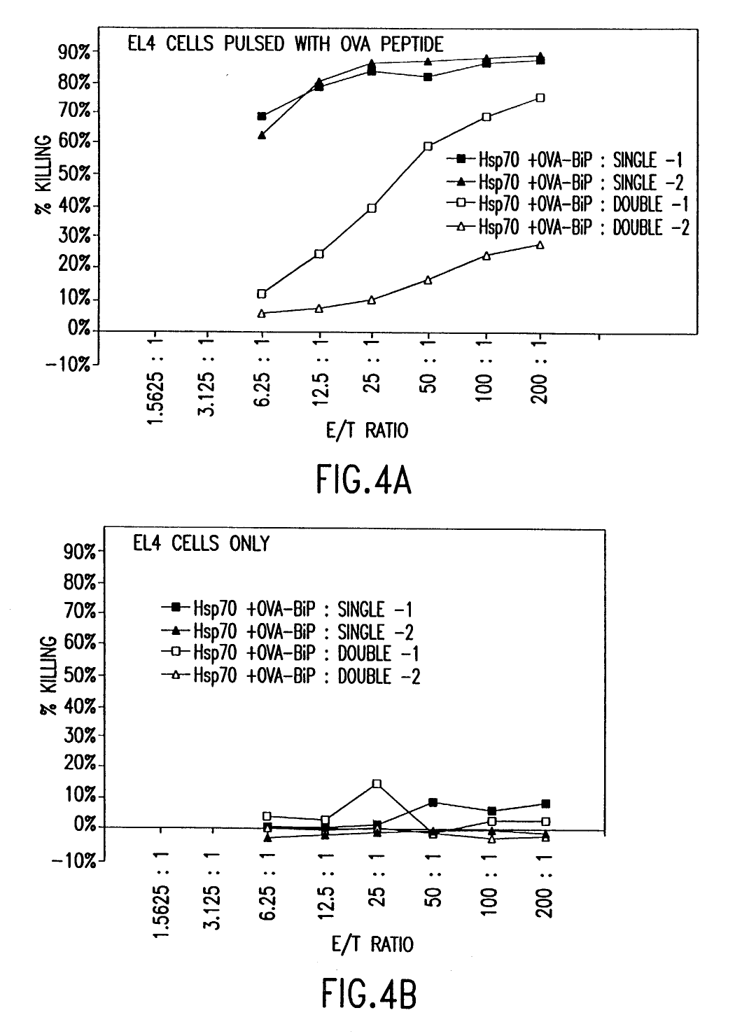

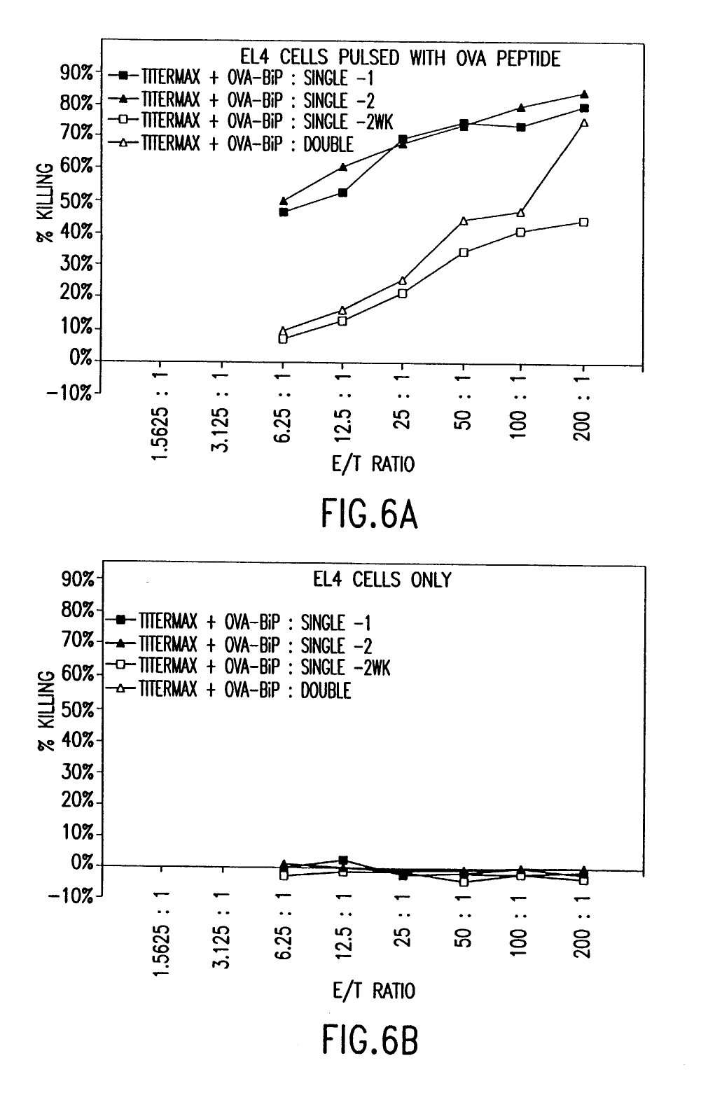

FIG. 4A-B shows the results of immunization of mice with hsp70 plus OVA-BiP conjugate peptide. Each curve represents data obtained from a single mouse. Mice were either immunized once (solid squares and triangles) or twice (open squares and rectangles). Percent killing of OVA-primed EL-4 target cells (FIG. 4A) or unprimed control cells (FIG. 4B) was measured. As shown in FIG. 4A, a single immunization with hsp70/OVA-BiP was able to induce an OVA-specific cytotoxic immune response which appeared to be greater than that induced by TiterMax/OVA (FIG. 3A) and as least as good as that induced by TiterMax/OVA-BiP (FIG. 6A). Mice receiving two immunizations appeared to manifest a somewhat smaller response. A similar response was obtained when mice were immunized once or twice with TiterMax/OVA-BiP (FIG. 6A).

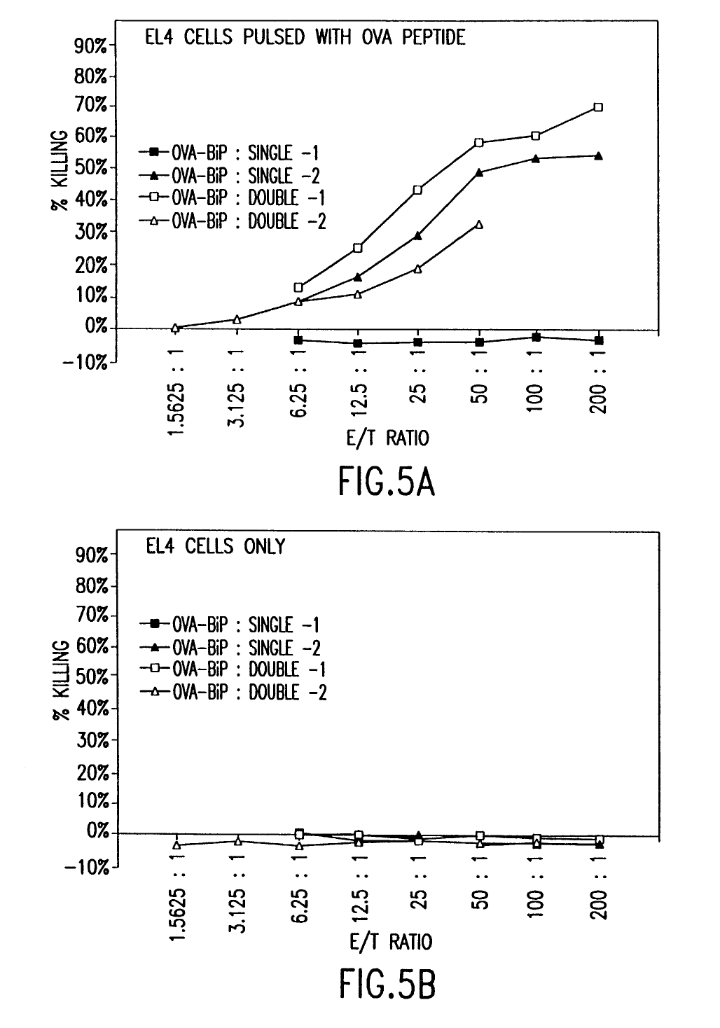

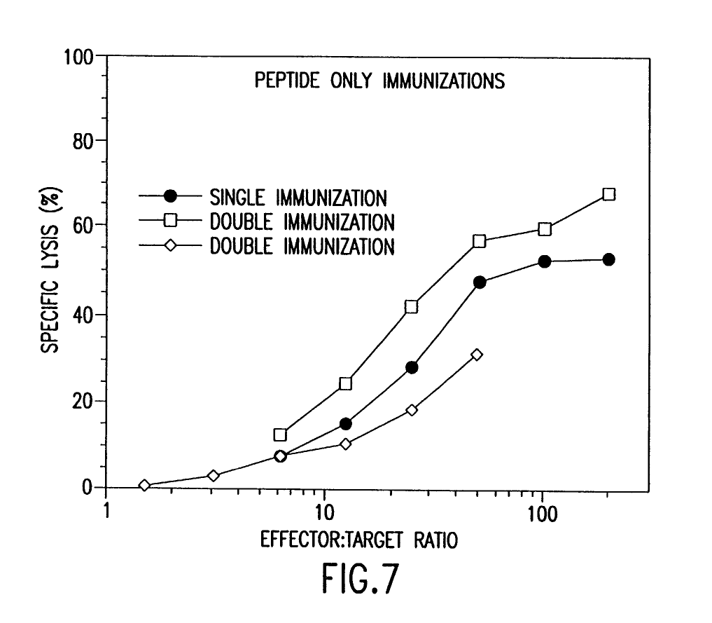

Interestingly, mice immunized with OVA-BiP alone were also found to exhibit a significant anti-OVA immune response, as shown in FIG. 5A. Effector cells produced from mice immunized once or twice with the conjugate peptide alone were tested against OVA-primed EL-4 target cells, significant cell lysis occurred (relative to lysis of naive EL-4 cells, as shown in FIG. 5B). Thus, the conjugate peptide OVA-BiP was capable of eliciting a cytotoxic immune response in the absence of added adjuvant. FIG. 7 shows the results when mice were immunized once or twice with OVA-peptide alone.

### 8. EXAMPLE: IMMUNIZATION WITH CONJUGATE PEPTIDE REDUCES TUMOR PROGRESSION IN VIVO

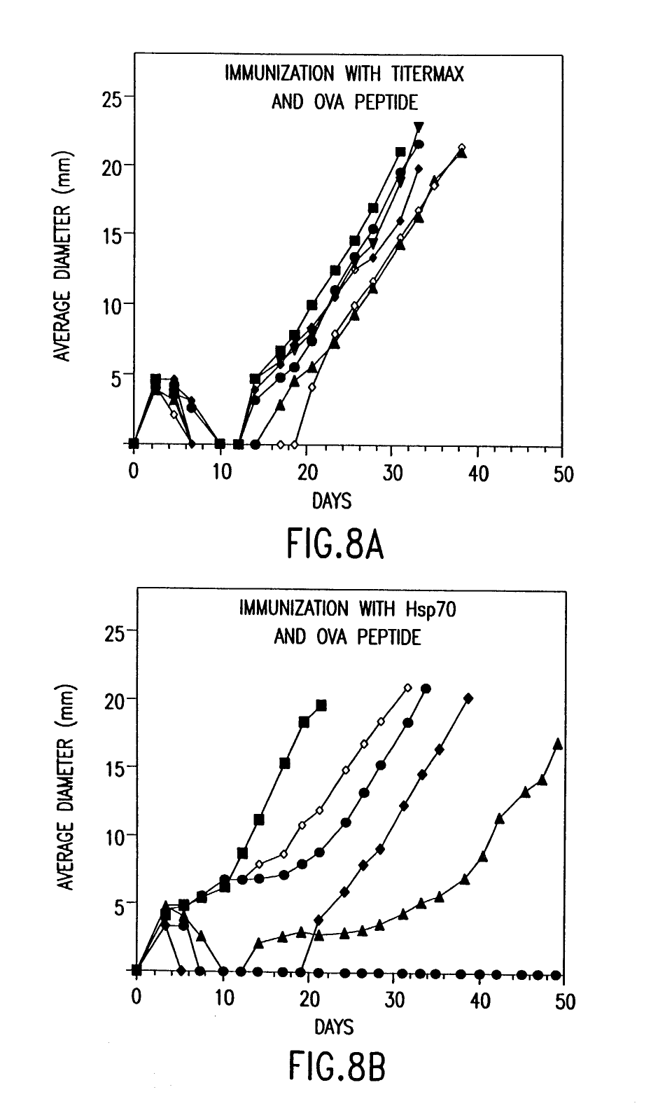

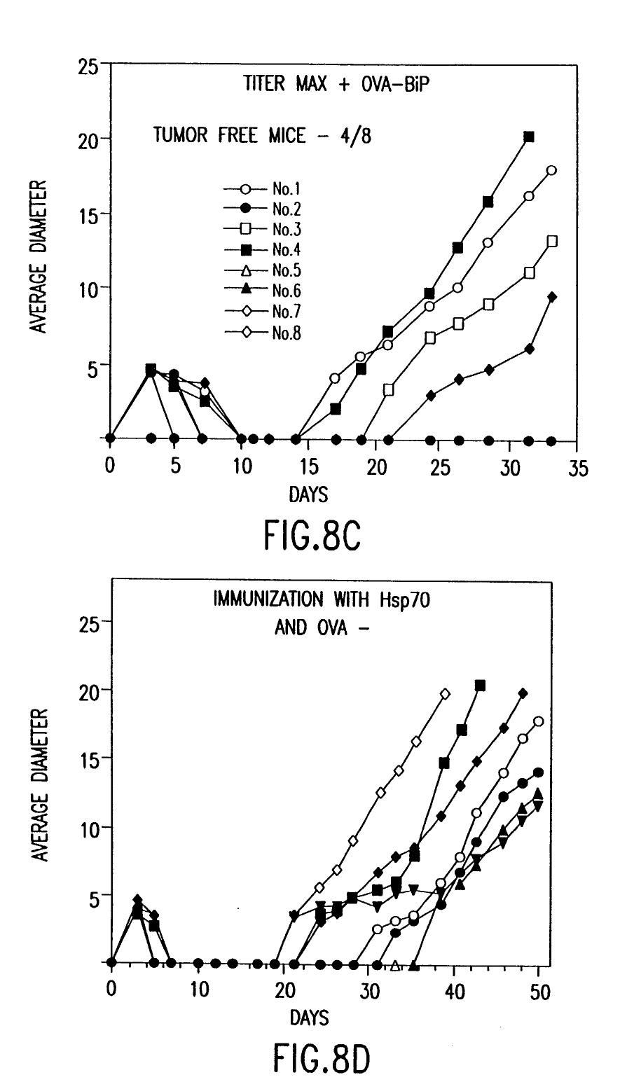

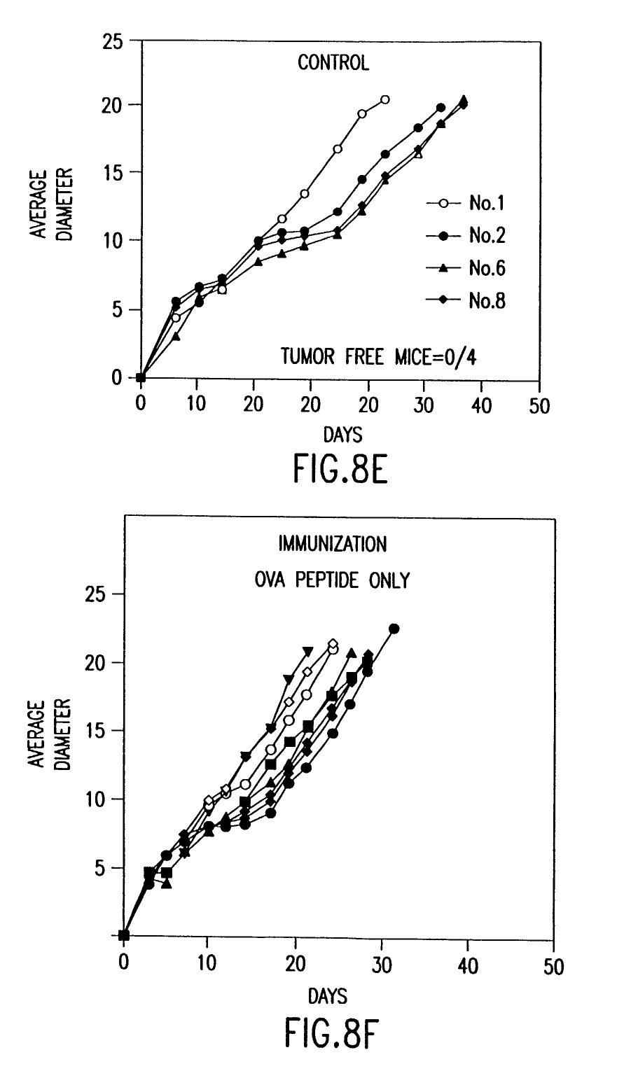

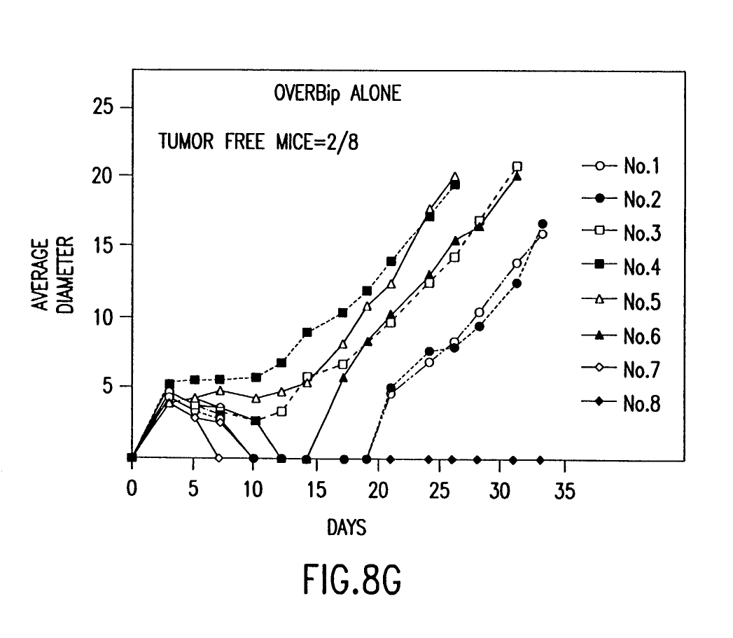

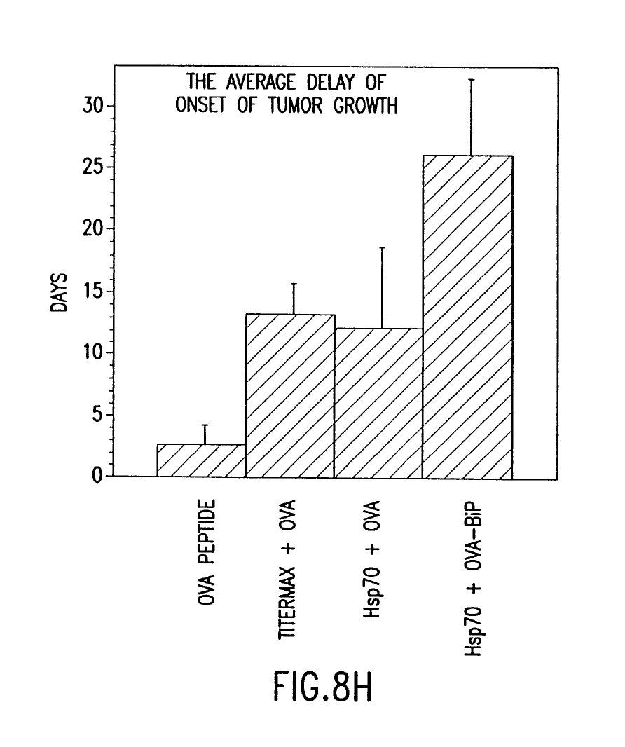

C57BL/6 mice, 8-10 weeks old, were immunized intradermally with one of the following (eight mice in each group): (a)5 μl TiterMax and 5 μg OVA peptide; (b) 15 μg hsp70 and 5 μg OVA peptide; (c) 5 μl TiterMax and 12 μl (OVA-BiP); (d) 15 μg hsp70 and 12 μg OVA-BIP; (e) control (four animals only in this group); (f) 5 μg OVA peptide; or (g) 12 μg OVA-BIP. The mice then were injected with 4×10⁶ EG7 cells. Tumor size was evaluated over time by measuring two diameters, the greatest diameter and the diameter perpendicular to the greatest diameter, and then calculating the average diameter. The results are shown in FIG. 8A-G (corresponding to groups a-g, as set forth above).

The data indicate that when administered with TiterMax adjuvant, OVA-BIP (FIG. 8C) was superior to OVA peptide (FIG. 8A) in reducing tumor diameter and in preventing detectable tumor formation altogether. Further, tumor size in mice immunized with hsp70 and OVA-BIP (FIG. 8D) was less than in mice immunized with hsp70 and OVA-peptide (FIG. 8B). In mice receiving peptide alone (without TiterMax or hsp70), while no animals were tumor-free when OVA-peptide was the sole immunogen (FIG. 8F), 2/8 animals immunized with OVA-BIP were tumor-free and the average tumor diameters were smaller (FIG. 8G). It therefore appears that the conjugate peptide associated with hsp70 was more effective than the antigenic peptide alone at preventing or reducing tumor formation in vivo (FIG. 8H).

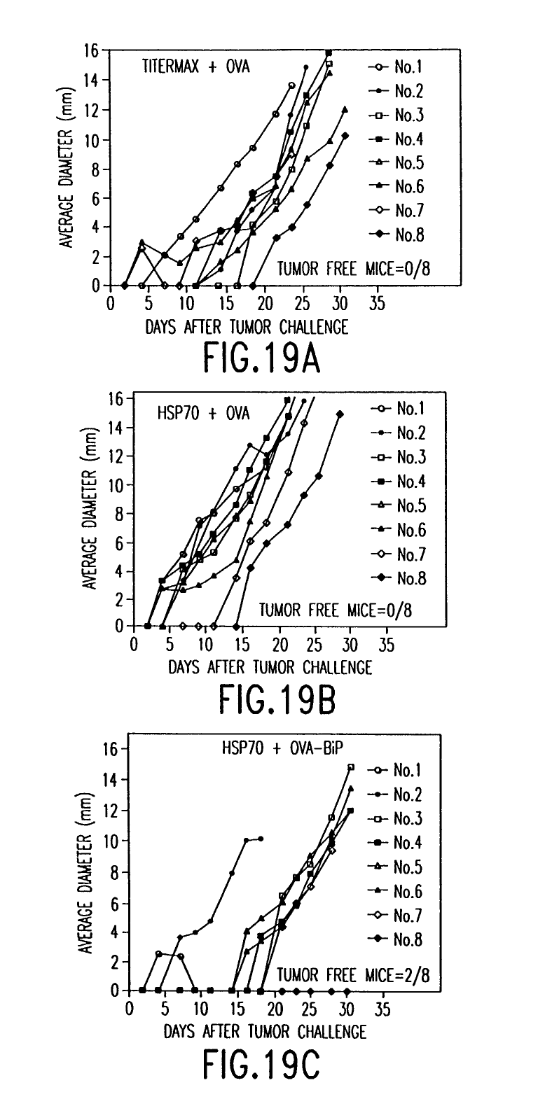

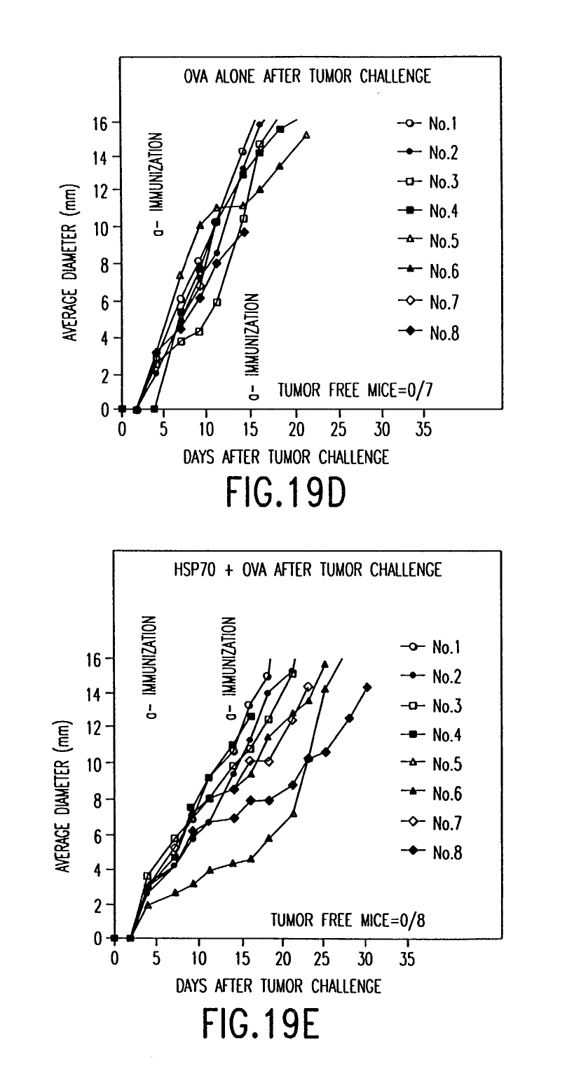

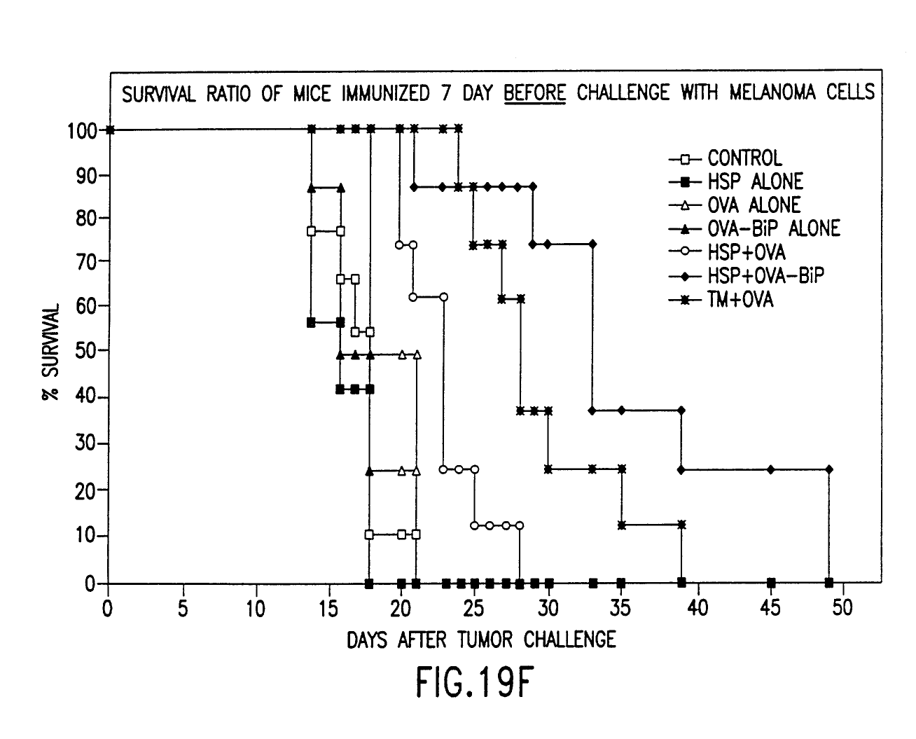

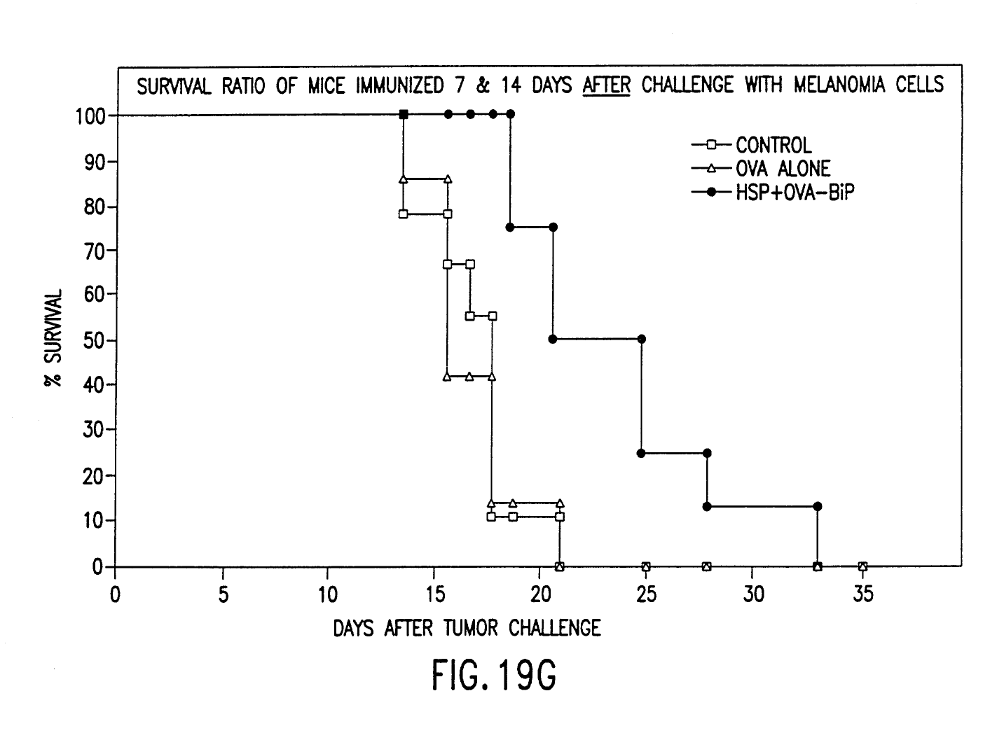

FIGS. 19A-E show the results of analogous experiments in which mice were challenged with a second tumor cell line, namely the ovalbumin-expressing melanoma cell line MO4. Mice were immunized with either (A) 5 μl TiterMax plus 5 μg OVA peptide, (B) 15 μg Hsp70 plus 0.5 μg OVA peptide, or (C) 15 μg Hsp70 plus 1.2 μg OVA-BiP seven days before challenge with 1×10⁶ MO4 cells. FIGS. 19A-C show tumor growth over time, measured as the average tumor diameter for groups of mice A-C, respectively. FIGS. 19D-E show the results of experiments in which mice were first challenged with 1×10⁶ MO4 cells to establish a palpable tumor before immunization (fourteen days after challenge) with either (D) 5 μg OVA peptide alone or (E) 15 μg Hsp70 plus 1.2 μg OVA-BiP. FIGS. 19F and 19G show, respectively, the survival ratios of mice immunized seven days before challenge with melanoma cells and the survival ratios of mice immunized seven and fourteen days after melanoma tumor cell challenge.

As shown above with the EG7-OVA tumor model, Hsp70 plus OVA-BiP immunization conferred superior protection against MO4 tumor growth relative to immunization with either TiterMax plus OVA peptide or Hsp70 plus OVA peptide. Two of eight mice immunized with Hsp70 plus OVA-BiP were free of tumor, whereas none of sixteen mice immunized with either TiterMax plus OVA peptide or Hsp70 plus OVA peptide were tumor free. The same trend was observed when immunization occurred after tumor challenge; that is to say, tumor growth was slowest in the Hsp70 plus OVA-BiP immunized group.

Various publications are cited herein, the contents of which are hereby incorporated by reference in their entireties.

What is claimed is:

1. A method of identifying a peptide which binds to a heat shock protein, comprising:
(i) contacting a phage display library comprising a plurality of bacteriophage which express, in a surface protein, a plurality of inserted peptides with a hsp target in a physiologic binding buffer;
(ii) isolating a phage which binds to the hsp target; and
(iii) identifying the inserted peptide expressed in the surface protein of the phage.

2. The method of claim 1, wherein the ionic strength of the binding buffer is equivalent to the ionic strength of an aqueous solution of 100-150 mM NaCl.

3. The method of claim 1, wherein the binding buffer comprises calcium ion at a concentration of 1-25 millimolar.

4. The method of claim 1, wherein the binding buffer comprises a reducing agent.

5. The method of claim 1, wherein the binding buffer comprises a non-hydrolyzable nucleotide.

6. A method of identifying a peptide which binds to a heat shock protein, comprising:
(i) contacting a phage display library comprising a plurality of bacteriophage which express, in a surface protein, a plurality of inserted peptides, with a hsp target bound to a benzoquinone ansamycin antibiotic, in a binding buffer;
(ii) isolating a phage which binds to the hsp target; and
(iii) identifying the inserted peptide expressed in the surface protein of the phage.

7. The method of claim 6, wherein the benzoquinone ansamycin antibiotic is herbimycin A.

8. The method of claim 6, wherein the benzoquinone ansamycin antibiotic is geldanamycin.

9. The method of claim 6, wherein the binding buffer is physiologic.

10. The method of claim 9, wherein the ionic strength of the binding buffer is equivalent to the ionic strength of an aqueous solution of 100- 1 50 mM NaCl.

11. The method of claim 9, wherein the binding buffer comprises calcium ion at a concentration of 1-25 micromolar.

12. The method of claim 9, wherein the binding buffer comprises a reducing agent.

13. The method of claim 9, wherein the binding buffer comprises a non-hydrolyzable nucleotide.

14. A conjugate peptide comprising (i) a tether which comprises a peptide identified by the method of claim 1; and (ii) an antigenic peptide.

15. A conjugate peptide comprising (i) a tether which comprises a peptide identified by the method of claim 6; and (ii) an antigenic peptide.

16. A method of inducing an immune response in a subject in need of such treatment, comprising administering an effective amount of the conjugate peptide of claim 14.

17. A method of inducing an immune response in a subject in need of such treatment, comprising administering an effective amount of the conjugate peptide of claim 14 bound to a heat shock protein.

18. A method of inducing an immune response in a subject in need of such treatment, comprising administering, to the subject, a composition comprising a conjugate peptide, wherein the conjugate peptide comprises (i) a portion which may be bound to a heat shock protein under physiologic conditions and (ii) a portion which is antigenic, wherein a heat shock protein is not concurrently administered with the conjugate peptide.

19. A conjugate peptide comprising an antigenic peptide and a benzaquinone ansamycin antibiotic.

20. The conjugate peptide of claim 19, wherein the benzoquinone ansamycin antibiotic is geldanamycin.

21. The conjugate peptide of claim 19, wherein the benzoquinone ansamycin antibiotic is herbimycin A.

22. The conjugate peptide of claim 14, further comprising a benzoquinone ansamycin antibiotic.

23. The conjugate peptide of claim 22, wherein the benzoquinone ansamycin antibiotic is geldanamycin.

24. The conjugate peptide of claim 22, wherein the benzoquinone ansamycin antibiotic is herbimycin A.

25. The conjugate peptide of claim 15, further comprising a benzoquinone ansamycin antibiotic.

26. The conjugate peptide of claim 25, wherein the benzoquinone ansamycin antibiotic is geldanamycin.

27. The conjugate peptide of claim 25, wherein the benzoquinone ansamycin antibiotic is herbimycin A.

28. A method of inducing an immune response in a subject in need of such treatment, comprising administering an effective amount of the conjugate peptide of claim 19.

29. A method of inducing an immune response in a subject in need of such treatment, comprising administering an effective amount of the conjugate peptide of claim 22.

30. A method of inducing an immune response in a subject in need of such treatment, comprising administering an effective amount of the conjugate peptide of claim 25.
